(19) United States
(12) Patent Application Publication
(10) Pub. No.: US 2003/0188740 A1
(43) Pub. Date: Oct. 9, 2003

(54) Method for production of enhanced traceable immunizing drinking water and other liquid and gas products, devices for production and use thereof, and use of the enhanced products for immunizing living beings

(76) Inventors: Zamir Tribelsky, Mevasseret Zion (IL); Michael Ende, Moshav Shoeva (IL)

Correspondence Address: LOWE HAUPTMAN GILMAN AND BERNER, LLP, 1700 DIAGONAL ROAD, SUITE 300 /310, ALEXANDRIA, VA, 22314, US

(21) Appl. No.: 10/258,349

(22) PCT Filed: Apr. 27, 2001
(86) PCT No.: PCT/IL01/00383

(51) Int. Cl. A61M 11/00; A61K 33/00

(52) U.S. Cl. 128/200.14; 424/600

(57) ABSTRACT

A method for the production of enhanced traceable optp-physiological polished liquids, and gases or solids or combination for immunizing living beings, devices using the method, use, and preferred mode for utilization are disclosed. A multi processing platform is proposed according to the invention harnessing time domain optronics of light & sound, wherein the transient sound produced by light is measured, referenced or calibrated against the light produced by sound for the formation adequate energy levels or densities or fluence rates for the purpose of dissociation of noxious or innocuous species or combination constituents components while keeping their geometrical integrity above their predetermined resonance levels, thus intact for later traceable recognition and triggering of positive decisive action by immune systems.

### FIELD OF THE INVENTION

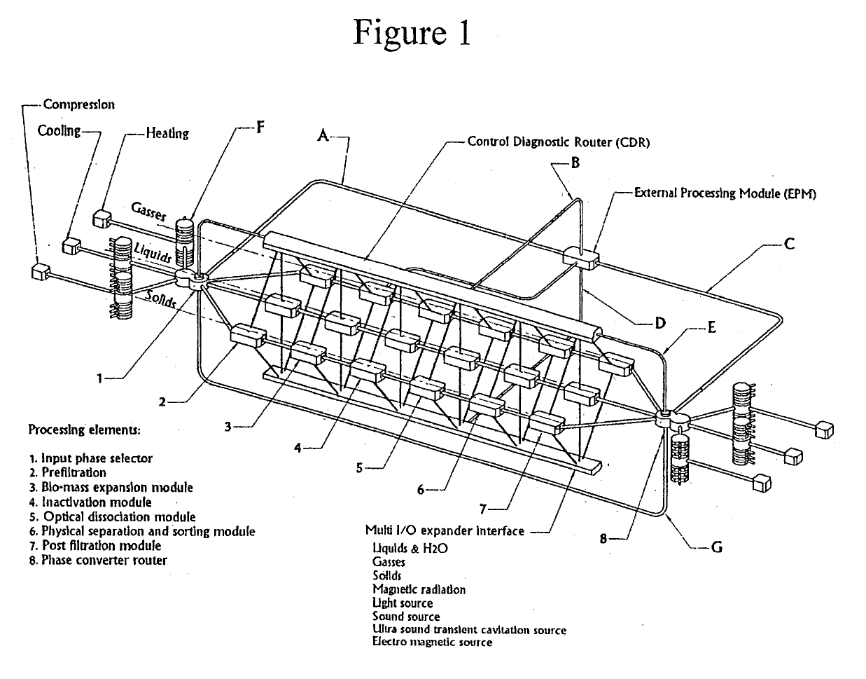

The evolution of the Human Immune System (HIS) have been studied for many years with great expansion of knowledge covering both the inherent, active and the acquired immune system portfolio of potential definitive reactions. Further more, the present invention discloses a novel methodology for the production of a synergetic bio-compound which includes among other ingredients, at least one protein mixture, or membranes mixture, or photo-damage products for making a predetermined compounding volume which when consumed by Human, Animals, and plants causes stimulation and enhancements to the Human Immune System (HIS). Further more, such bio-compound is herewith disclosed for the enhancements of the immune system by for example lubricating, tuning, reformatting, and/or positively enhancing the influences of the Major Histo-Compatibility Complex reactions, and the Immunoglobuline production, and more specifically, such enhancements, will augment the flow rate of Immune system components and their distribution, or their availability at a given location (through out the human body) and in places wherein a threatening infectious intakes may occur. Such positively increased level of signaling, triggering, enhancing, and stimulating the immune system according to the methodology disclosed in the present invention, provides a single platform in which naturally occurring interception of antibodies and spontaneously occurring infectious threats, are being dealt with, decisively, thus, while facilitating the education of the immune system for swift and decided responses by consumption of drinks, or by breathing in air (i.e. the present methodology could be used for both: liquids and gasses). More specifically, the present invention allow for setting a threshold, or a velocity curve for the immune system to act upon, or be reverenced-by, thus by feeding it sufficient compounded signaling, or chemical traceable photo-biological substances which once been translated upon consumption, leads for setting a point of. reference from which the immune system act from decisively, and positively, for remedy, maintenance, and operation verification and rehearsing of the immune system for example: It is already known that water is one of most precious resource on our planet, all living beings, Human, animals, fish, sea creatures, and plants and trees, they all indeed must have water and are able to consume, utilize, and process said water, to a predetermined specie specific calibration standards, or capability. Further more, water is herewith proposed, to be processed according to the methodology of the present invention, polished by the interaction, and interference's of energies (according to the invention herewith) and made to enhance the immune system of consumers (FIG. 1, a-z-12). More specifically, when the water are treated with the various photonic, mechanical, photo-chemical, Photocatalysis, electro-catalysis, sonication, and acoustic energy, thus interfering, and interactively pass through the (1) stages, (2) spheres, or (3) states in the modules, conduits, or chambers of devices according to the present invention, then these water will have contained sufficient proteins traces, and traceable signaling by means of chemical highlights, and thus triggers for the enhancement of the end user, the individual person, the consumer own immune system. Once been consumed said biologically interactively enhanced is causing immune actions to be revitalized, prompting libido and prana expand for improved health, and enhancing the preparatory phase in which an individual body state tuning, and referencing has improved.

### BACKGROUND TO THE INVENTION

Scientist engineers bio-technologists, and producers and end users where for many years puzzled by the opulence of the immune system's resources. More specifically, although it is already known that the Human body could recognize and mount unique responses to vast varieties of (externally) invading threatening antigens—but how these processes are governed, and not all of their specie specific calibration standards have been understood. Further more, strict limitations exist as to the knowledge for the assimilation and command controlling of some “education” procedures relating to the programmability and the remarkable poly-abilities of the inherent, active and acquired modular orientation, recognition, marking, and/or sorting and/or signaling or triggering of the Major Histo-compatibility Complex (MHC type I, II) especially beneficial for the production of ammunifying poly-bio-compound to be held contained, mixed and/or integrated within liquids and gasses or solids or combinations for economical globalization of mass transport and distribution intakes by population for the purpose of protection and strengthening or vitalizing of immune system operational, maintenance and production performance.

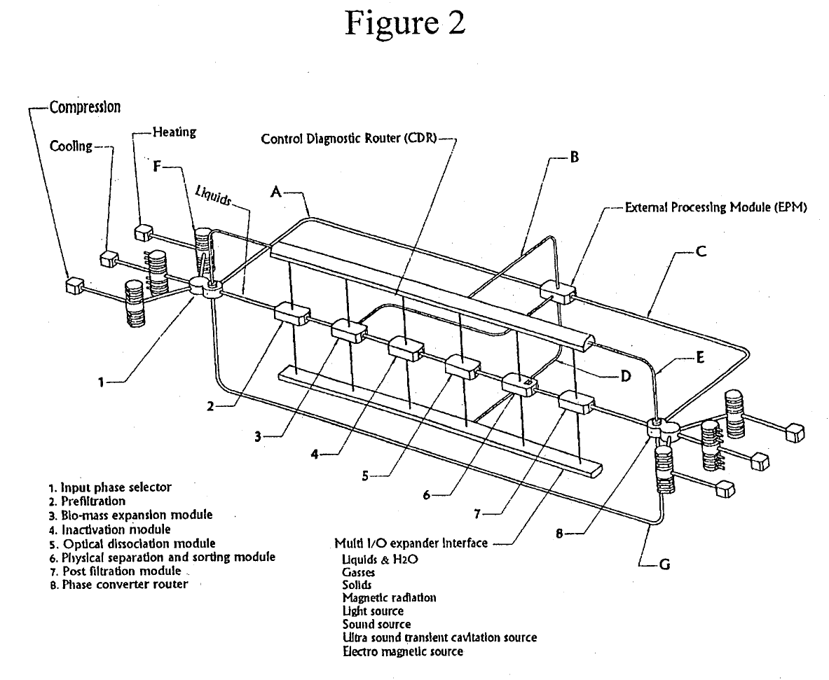

More specifically, these limitations have prevented an socio-economical hybrid solution or methodologies wherein a single production method or platform (Such as disclosed & referenced in FIG. 1, 1-8/a-z of the present invention) could account for the highly diversified varieties of potential infectious events by means of a simplified and streamlined continuous process, more specifically, no prior art in the field offer to date, a method in which both producers and end users may assimilate the knowledge derived from the present invention and economically apply it for its production. In addition to its valuable environmental protection capabilities, the present invention offers a complete solution against the natural evolution of noxious species (i.e. for example: Bacterium, Viruses, Pathogens) and new challenges thus continuously introduced to the Human immune system. More specifically, current knowledge has not yet being extended to cover the production of a single specie specific signaling calibration standards for the production and/or synthesizing of a polybio-compound, such as described in the novel methodology of the present invention (FIG. 1, 1-8 a-z of present invention, FIGS. 2-12, ref. 1-100).

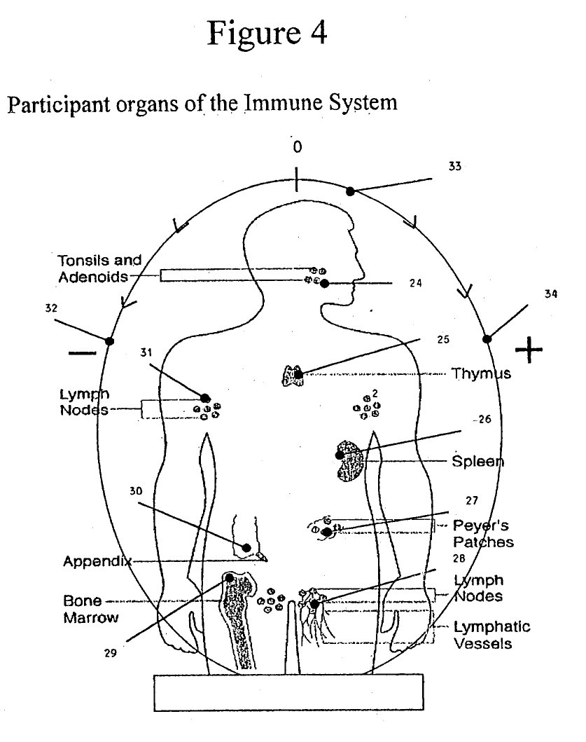

Further more, A method for the production of a predetermined volume of photo inactivated, Bio-reacted, Polyphonically resonating vapor propelled for harmonious proportioning of DNA, & RNA for the apportioning (FIG. 1, 1-8/a-z, FIGS. 2-12, ref 1-100) purpose of furnishing the world of Man, Animals and plants with a powerful water based or air based or solid based [(FIGS. 1-12), (1), (7), (1-8/a-z), (9), (10), (11), (12), (ref. 1-100)] Bio-compound enriched with a continuum of biological substance having an origin or traces of at least about 1 replication sequencers triggering, stirring and educating chemical, photochemical, electro-photochemical, electro-photo-catalyzed, and photo-catalyzed, and/or electrolyzed predetermined volume of modulators, conditioners, and/or triggering chemo-electrical signal generator enhancing and strengthening, expanding, programming, and revitalizing, accessing and retrieving positive bio-immune reactions stabilizing and path finding facilitating streamlined free and UN—hindered flow rate, positively effecting immune components distribution time (FIG. 4, ref. 34+/−), and velocities within a predetermined space or area of the body over a predetermined period of time.

Current methods and means for the production of synthetic, and or natural or combination of modulators, controllers, and/or triggers for the immune system have failed to provide a complete and comprehensive repeatable solution against global varieties of threatening and constantly evoluting noxious species. Further more, never has yet being available a single frame work for the production in situ or for the pre-post-production of a potent Polly-bio-compound, such as in the methodology of the present invention, and said method for its economic manufacturing is aimed at positively enhancing the environment in which we live-in, aimed at, and for the purpose of ammunifying the immune system of Human and Animals, and plants through such global, accessible, and universal distribution platform reaching all populations on earth—Drinking water, and through the enhancement of such water as an environmental protection tool, the method of the present invention, enhance and protect public health, conserve energy, and may be used for wide varieties of beneficial production applications, especially beneficial for the furnishing of enhanced drinking water, and breathing air (liquids and gasses) as proposed in the method according to the present invention.

The current limitation imposed by known method and means for the natural, and synthetic production of antibodies have failed to implement economically a global solution for an accepted efficient solution and have left large population segments exposed to the threats of random, cyclic, Recurrent or non recurrent eventualities in which through out domestic, industrial, agricultural and medical domains—aspects of the immune system operates unresolved, without vital controllability or without the necessary tools for performing re-calibration, referencing, rehearsing and educating of the immune system (i.e. both active, inherent, and the acquired types or activity characteristics) and its relevant associated MHC (1, 2, 3) (Major Histo-Compatibility Complex).

More specifically, The novel methodology of the present invention fulfill the purpose of attuning, and/or accelerating, and/or rehearsing Human Immune System (HIS) Immune System reaction times, intensity curves, and flow rates and distribution, production efficiencies, availability and global, individual's body distribution of (such as Immune-globulin's) and enhance their respective transport velocities, that is why the method according to the present invention is not so limited, that's why the present invention may provide an ammunifying solution for wide variety of applications wherein liquids and/or gasses and/or solids or combination is being consumed, taken, produced and integrated into vast varieties of products.

For examples, Many allergic reactions which have been recorded around the world have left doctors, scientists, engineers and bio-technologists as well as end users puzzled (i.e. from the opulence of the Human Immune System portfolio of reactions), many issues relating to the flow rates. of immune (immunoglobulines)-globulin's production and equipping rates have been left UN attended or without clear and concise answers, and/or solutions for the stabilizing and synchronization of an individual Human, Animals, and plants immune system reaction strings. More specifically, the present invention is not so limited, that's why the novel methodology of the present invention facilities the ability. to synthesize specie specific calibration standards for lubricating, and enhancing the operation, aspects, and capabilities of a particular Immune system to deal with a predetermined bacteria, viruses, and other toxic biological and non biological accumulating compounding volumes.

(Examples of Prior Art and Their Limitations)

Example of prior art in the field is herewith presented in order to illuminate and emphasize the inventive step, inventive progress and commercial applicability and benefits using the novel methodology of the present invention. It is also important to note that water for drinking and air for breathing are often contaminated by wide variety of toxic compounds (such as Selenium, Boron, Bromates), these compounds must be removed to adhere to standards, maintain flow through of blood in vessels, water in pipes, or conduits, and/or chambers, or facilitate washing, cleaning, and removal of noxious particles, and toxic compounding volumes. The present invention also provide a beneficial mechanism for the removal of such toxic compounds (Selenium) from water by photocatalytically, and electronically manipulating the composition, and thus the associated molecular structure (example from Selenium IV-Selenium 0, or Selenium VI, Boron, or other toxic compounds such as Bromates and others. The beneficial mechanism of the sub-microsecond time duration. methodology of the present invention facilitate the formation of larger particulate materials having electronic composition, molecular weight, and some often float on water after exposure to pulses in the UVC having each time domain in the Sub-microsecond time duration's. Fresh, mineral, suspended, mixed or flavored water or vapors could be composed that way using the methodology of the present invention Selenium may be removed by conventional filtration or scraping arms, and cleaned water thus may be extracted at lower physical levels wherein the floating Selenium is picked up, and removed for later processing of line, leaving the waters free of toxic chemicals and ready, and biocompatible for preparation of traceable compounds in water, air, liquids, and gasses. When such beneficial advantages are facilitated according to the methodology of the present invention. Itr is further noted that no methodology currently in use offer a single platform for treatment, and preparation of enhancing liquids, and gases offer the ability to prepare, treat, and process a multiplicity of processes all synhergetically arranged sequentially, or simultaneously, or cyclically, or non recurrently.

The examples presented does not intend to limit the scope of the present invention what so ever, further more, this examples (patents: ADT WO-9958948 A2 1999WO-US 10917 19990513 and patent: PI WO9958948, DNN N2000-D41379, DNC C2000-013819 illustrates the unresolved need for such single electro-triatmic or electromechanical Opto-acoustic, and/or Acousto-optically combined and/or integrated multi-processing platform or production methodology according to the present invention.

More specifically prior art in the field have led to the implementation of diagnostics and analytical methodologies and devices and their scope does not cover production of such polybiological compound (such as in the present invention) and although beneficial, these prior art methodologies may assist the present invention but not productively replace or avail alternative to the method of the present invention. Further more, Prior art in the field of diagnostics and analytical methods for compounding and analyzing biological substances (i.e. such as DNA, RNA) portray the technological evolution which led to the present invention method for the production of a polybiocompound and devices which uses, and utilizes this method. Specifically In contradistinction to prior art in the field, ADT WO-9958948 A2 1999WO-US 10917 19990513 This patent describe method for analytical measurements aimed at capturing and analyzing predetermined amount of analyte. This methodology does not interfere with the novelty of the present invention but rather show the luck of methodology (such as disclosed by the present invention) for the production of a polybiological compound whether water, air or solid based or mixed or any combination thereof.

In contradistinction to prior art for example: AN 1999-443884 (37) WPINDEX DNN N19999-331070, DNC C 1989-130702, DC A18, A23, A26, A96, B04, D16, D22, E13, J04, P75, S03 in the field of biosensors used to detect analyte, and to potentially quantize analyte's amounts. As this type of scope (N1999-331070, DNC C 1989-130702, DC A18, A23, A26, A96, B04, D16, D22, E13, J04, P75, S03) cover the detection of such substances as chemical or biological contaminants amount, concentrations or contamination in garments (e.g. drapers, curtains, rugs, sheets etc.) and is therefor limited to analytical applications or quantity measurements. Strict limitations are imposed to the ability of prior art to process or convert across different phases (as an example: such as from water to air visaversa or mixed combinations) while actually producing beneficial polybiocompound for strengthening of the immune system.

Further more it is impossible to produce a biologically conditioned air for breathing or water for drinking using the analytical methodologies of prior art (mentioned above: N19999-331070, DNC C 1989-130702, DC A18, A23, A26, A96, B04, D16, D22, E13, J04, P75, S03 in the field of biosensors used to detect analyte, and to potentially quantize analyst's (or analyte) amounts. As this type of scope (N1999-331070, DNC C 1989-130702, DC A18, A23, A26, A96, B04, D16, D22, E13, J04, P75, S03), more specifically, by definition, the present invention is not so limited.

U.S. Pat. No. 5,364,645, by Lagunas-Solar, et al, A method for controlling microorganisms by pulsed ultraviolet laser radiation”, this invention is speaking on reducing population, or eliminating bacteria, on the surface of fruits, in contradistinction to the present invention, here only disinfection (i.e. inactivation of DNA, and/or RNA replication sequences), no biomass expansion was done previously, no time stamp given to batches, or predetermined volumes, no address or mentioning of the immune system ability to recognize, nor is the invention able to produce biologically enhanced water, or air, the present invention is disclosing a method for disinfection, or inactivation, but no selection of source, species, specifics, and no disclosure on production of biologically enhanced drinking water, the resulting water processed by Solar patent (U.S. Pat. No. 5,364,645), or fruits, or surface of foods, or anything processed by this (Solar) invention will never have sufficient amount for triggering the immune system, or there may be sufficient amount of element while UN sufficient amount or volume from another component, further more the dissociation, and optical, electrooptical, photochemical, Photocatalysis, electrocatalysis, of the method according to the present invention make sure that the proteins, membranes and other remaining substances are dissociated, separated and sorted in order to create such biologically enhanced and polished, water, or air, that's why the present invention is much less limited and is clearly taking a further step, beyond disinfection, and into dissociation, separation, sorting, and polishing, more specifically the present invention is more advance, more capable of providing a beneficial drinking water and air for the human immune system, or for animals, or for plants, or for liquids and gasses, and wherein the U.S. patent, number U.S. Pat. No. 5,364,645, will not be able to perform the same process in air, or to make sure there is sufficient proteins, or components which the immune system could easily recognize, further more, there is no step to preserve the remains, triggers, signals, or chemical structure, and formation (such as in the present invention), but rather the objective of this invention (Solar), is to reduce, or eliminate the microorganism, in contradistinction, our invention (the present invention) is less limited, and could be thus used for vast myriad of application, especially beneficial for the human immune system, and for organs, or cells of the immune system, more specifically the present invention can tune, i.e. the paten of the present innovation of the invention.

the present invention herewith discloses a novel methodology for the economic production of a polybiocompound to be consumed for the enhancement of the immune system. More specifically that's why the present invention could be used for wide variety of applications in domestic, commercial, Agricultural, medical, food and drinks industries, mineral water output of aquifers, spring water and vitamin water bottling and productions as well as aqua cultured such as fisheries, and protection of public health through as well as mass production of any water or air based products. and in any product which during its composition the liquids and gasses or solids or combination thereof may be passed through partially or wholly and processed by the production methodology of the present invention. For example; Such diversity of applications may be using the method according to the present invention for the production of biowatered (in droplets) or vapor in air or drinking water, or mineral water wherein the phase (Liquids) has been selected for reception for processing into/by the system and is also appear at the end of systems according to the method of the present invention as water. (Same phase, water). Or alternatively phased water could be selected at system input but converted to gas or vapor at the system output according to the present invention vaporizing could be perform using transient cavitation or ultrasound, or by predetermined mono, and/or polyphonic vibrations (from about 1 Hz- to about 218 Mhz, or mechanical (by centrifuge etc.) or by pressure hydraulically or pneumatically or in any combination thereof. Further more the (b) system according to the method of the present invention may include: (a) A compressor, (b) a pump, (c) a pneumatic I/Os, or (d) an Acousto optical interface, (e) or optical modulation interface or I/Os, or (f) a flow reducer, (g) an hydraulic wing for stirring, (h) an optical lens, or objective or difractive or grated or coated elements or combinations, (i) a congenital, or conventional quartz or High Grade Fused Silica (HGFS) optical waveguide or bundle of guides or harness operating from a multi tail common end termination to at least one additional guide or extension,) (j) A photonic band-gaped fibers or bundle, or network for the provision of high intensity source of light, (k) at least one laser system, (l) At least one flush lamp or Continues wave (CW) lamp with emission from the range of about 40 nm to about 2500 nm range, (m) at least one reactor, module, tank, or container, chamber or conduit type, (n) At least one spectral acquisition unit having a networking interface, (o) At least one hydra-pneumatic bridge interface or I/Os, (p) at least one drop of water, (q) At least one volume or breath of clean air, (r) at least one predetermined volume of compressed biocompound in compressed air, at least one measure of bio-water in liquid drink or frozen or delivered and transferred through agriculture and irrigation's, or dripping pipes to vegetables or fruits or any combination thereof holding, said water and preserving its relevant quality parameters, (s) T102 Photo catalyst, (t) Electro catalyst such as I.T.O+T.I.O.2, (w) A white light source having a high intensity source of polychromatic light, (x) A monochromatic light source having a high intensity source of pulsed or continuous wave type of light, (y) An accurate clock for the control command and programmability of cyclic, automatic, recurrent, and non recurrent operation modes, (z) 1^{st}2^{nd}3^{rd} generation Human consumers and their (ready to be enhanced) immune systems, thus the present invention provide for global beneficial update in the evolution of flues and other diseases as well as toning and tuning defense reaction of the immune system. More specifically, as an example: when the water are treated with the various photonic, mechanical, photo-chemical, Photocatalysis, electro-catalysis, and pass through the stages, spheres, or states in the modules, conduits, or chambers of devices according to the present invention, then these water will have contained sufficient proteins traces, and signaling triggers for the enhancement of the end user, the individual person, the consumer own immune system action revitalized, and libido and prana expand for improved health and preparatory body state tuning, and referencing.

the present invention discloses a novel methodology for the production of polybiological compound which could take the form—of, or be integrated into liquids, or gasses, or solids. More specifically and in contradistinction to prior methods and means which only provide analytical techniques and devices, the novelty of the present invention to produce a unique water or air based compound for human consumption is derived, principled and propelled from its ability to offer a continuum processes in which the flow chart order or stages or phases could be altered, and/or be converted or modulated while keeping the processes velocities or characteristics intact. Especially beneficial for enhancing the Human Immune System through drinking of Biowater (i.e. water having biological traces or signatures therein, made safe then conditioned for later recognition.

More specifically the inventive step, modularity of the processes and the entire methodology of the present invention may use analyte or analytical techniques only to enhance its own control mechanisms or locate cycles in which referencing should be obtained, and/or maintenance must be obtained, or to analytically determined the types of polybiological compound produced originally by the method of the present invention but the present invention does not rely on any prior art in the field, the present invention discloses an original production method keeping all natural characteristics, and/or processes in their original form facilitating the rehearsing and overall tuning ability of consumer's immune systems responses and defense and ramediating reactions.

### SUMMARY OF THE INVENTION

It is very beneficial and preferred for Humans, animal and plants allowing for enhancements manufacturing methodology according to the present invention to produce for example biologically traced or residuum water or bio-air (or biosolids), or combinations which all or in parts positively effect the immune system promoting remedy orientated reactions from said liquids and gasses and solids or combinations for wide variety of domestic and commercial market applications as well as in agricultural and medical domains.

More specifically the novel methodology according to the present invention facilitating the production of drinks (FIG. 1, 1-8/a-z, of the present invention) and foods or agricultural produce (containing liquids, gasses, or solids) which strengthen the immune system whether served, stored, or prepared for consumption integrated with vitamins, minerals, nutrients, and/or any combination thereof. Such produce according to the present invention may include: Water, mineral water, spring water, Juices and beverages, syrups, flavored syrups enhanced with vitamins and protein traces according to the resent invention, or juices according to the present invention may be extracted from fruits watered, and/or irrigated by water produced processed or delivered through devices operating according to the novel methodology of the present invention.

The present invention relates to a method for production of biologically enhanced drinking liquid or breathing gas, useful for immunizing by traceable recognition properties of the immune system of living being or plant, comprising; exposing a predetermined volume of liquid or gas or a combination thereof having a predetermined quantity of biomass components having traceable biologically noxious source or origin therein, to a light radiation emerging from at least one high intensity light radiation unit, or to mechanical vibrations emerging from at least one mechanical vibration oscillator unit, for a predetermined time interval wherein the energy amount of said light radiation, mechanical vibration, or a combination thereof, is being sufficient for the dissociation agglomeration or coagulation of at least a part of said traceable biologically noxious source or origin and insufficient for altering their geometrical integrity for future traceability by the immune system of human or of other creature or of a plant, upon consumption.

The method of the present invention may further comprise the step of inserting a predetermined quantity of biomass components having traceable biologically noxious source or origin, into a predetermined volume of liquid or gas or a combination thereof, prior to exposing it to the light radiation or to the mechanical vibration.

The present invention relates also to a method further comprising the step of dilution of a predetermined volume of the liquid or gas or a combination thereof with a predetermined volume of other biologically enhanced or non biologically enhanced liquid or gas or a combination thereof.

A method for production of biologically enhanced drinking liquid or breathing gas further comprising the step of detecting a time stamp positioning mark of the liquid or gas or a combination thereof, as a quality checkout or in order to determine their immunizing characteristics or for calibration, is also a subject of the present invention.

The method for production of biologically enhanced drinking liquid or breathing gas, according to the present invention may further comprise the step or steps required for calibration of the light radiation properties by using a real time feedback of light originated within the treated liquid or gas as a result of the mechanical vibration activity, or calibration of the mechanical vibration properties by using a real time feedback of ultrasonic mechanical vibrations originated within the treated liquid or gas as a result of the light radiation activity, or any combination thereof.

The present invention further relates to a device for the production of biologically enhanced drinking liquid or breathing gas or a combination thereof according to the method of the present invention as defined in by this specification document. The device according to the present invention comprises at least one dissociation-energy generator unit adapted to generate the dissociative resonance of at least one biological, chemical, molecular, atomic, electronic, phononic, constituent within the liquid or gas to be enhanced; at least one chamber or piping means for receiving or for conduction of liquid or gas or a combination thereof wherein said liquid or gas or their combination can be exposed to energy emerging from said dissociation-energy generator unit; at least one control unit having means for determining an effective ratio between the volume of liquid or gas or a combination thereof, and the amount of dissociation energy to which they can be exposed, wherein the effectiveness of said ratio is useful for a dissociation of a quantity at least one of said constituents by said dissociation energy without altering its geometrical integrity for future traceability by the immune system of human or of other creature or of a plant, upon consumption.

More particularly, the biological constituent within the liquid or gas to be enhanced and to which the dissociative resonance of the dissociation-energy generator unit is adapted to, is selected from the group consisting of DNA, RNA, mRNA, lipid membranes or enzime.

According to one preferred embodiment, the dissociation-energy generator unit of the device of the present invention is comprised of at least one radiation unit containing at least one high intensity source of light. Preferably, the control unit has means for determining an effective ratio between the volume of liquid or gas or a combination thereof, and the amount of light radiation to which they can be exposed, wherein the effectiveness of said ratio is useful for a dissociation of at least part of said components by means of said light radiation, without altering their geometrical integrity for future traceability by the immune system of human or of other creature or of a plant, upon consumption.

According to another preferred embodiment of the device according to the present invention, the dissociation-energy generator unit is comprised of at least one mechanical vibration oscillator unit, and the control unit has means for determining an effective ratio between the volume of liquid or gas or a combination thereof, and the amount of mechanical vibration energy to which they can be exposed, wherein the effectiveness of said ratio is useful for a dissociation of DNA or RNA composite components contained within said liquid or gas by means of said mechanical vibrations, without altering their geometrical integrity for future traceability by the immune system of human or of other creature or of a plant, upon consumption.

The present invention further relates to another preferred embodiment of the device for the production of biologically enhanced drinking liquid or breathing gas or a combination thereof according to the method of the present invention, wherein the dissociation-energy generator unit comprises in combination at least one radiation unit containing at least one high intensity source of light, and at least one mechanical vibration oscillator unit; and wherein said liquid or gas or their combination can be exposed to mechanical vibrations emerging from said oscillator unit and simultaneously can be exposed to light energy emerging from said light source, within the chamber or piping means; and wherein the control unit has means for determining effective ratios between the volume of liquid or gas or a combination thereof, the amount and characteristics of mechanical vibration energy to which they can be exposed, and the amount and characteristics of light energy to which they can be exposed, wherein the effectiveness of said ratios is useful for a dissociation of at least part of the predetermined quantity of biomass components having traceable biologically noxious source or origin contained within said liquid or gas, by means of said mechanical vibrations and said light energy, without altering their geometrical integrity for future traceability by the immune system of human or of other creature or of a plant, upon consumption.

The mechanical vibration frequency generated by the dissociation-energy generator unit is between 1 Hertz and 1 Tera Hertz, and the light wavelength is between 1 nm and 3600 nm.

The sub mixing properties or rezonance and the total energy density zone or intensity or flouence rate or monofonic or polyfonic or vibration exatation level, or photo coagulation or agglomeration or transient or static cavitation or sonolysis of the said mechanical vibration and light radiation, are being able to cause dissociative molecular, atomic, electronic effects while keeping the integrity of single or multi component compound thus created according to the method of the present invention especially beneficial for tunning, referencing trigering stimulating enhancing, rehearsing and increasing the sensitivity, oppulance interconnectivity and flush or interoperability or programability, distribution and the flow of positive, accurate decisive actions and reactions of the NHC majore histo compatibility complex type I-II-III the aquired human immune system within a predetermined volume or period of time.

The present invention further relates to a device comprising feedback means for calibrating the light radiation properties by using a real time feedback of light originated within the treated liquid or gas as a result of the mechanical vibration activity, or feedback means for calibrating the mechanical vibration properties by using a real time feedback of ultrasonic mechanical vibrations originated within the treated liquid or gas as a result of the light radiation activity, or any combination thereof.

The present invention further relates to the use of biologically enhanced drinking liquid or breathing gas or a combination thereof produced according to the method as defined in this specification document, for immunizing living creature or plant.

By the use of the biologically enhanced drinking liquid or breathing gas or a combination thereof, post noxious bio-compound opto-physiological enhancements are being transferred or routed for programming or positively referancing charged suspension for rehearsing, and stimulating the acquired immune system, or for chemically equipping immune cells and organs of the human body upon consumption of A++ compound over a predetermined period of time

During the use of biologically enhanced drinking liquid or breathing gas or a combination thereof, at least one human, chicken, or cow, bird, or reptile, insect, or micro, and/or macro organism is consuming said biologically enhanced liquid or gas by spraying, washing, rubbing, dipping, wetting, or drying or said biologically enhanced liquid or gas is being consumed as food stuffs, still, or bubbling drinks, flavored, or enriched with minerals, or nutrients or oxygenation reducing substances for the purpose of enhancing the immune system through dietary schedules, and wherein said dietary schedules are aligned on time track for updating the immune system to potentially threatening infectious events or pending diseases, or for the purpose of preempting, securing, and making safe generations of crops, and animals, man and plants by consuming, in-taking, drinking, and breathing applying, and vaporizing, cleaning with, or soaking in deep, or shallow volumes, or, said biological enhancements according to the present invention calibrate the immune system through enhanced and accelerated production of immune system elements and/or cells, and or organs, and which a responsive, or decisive, or interactive pre-programmed, or predetermined, preset, or variably adjusted for the purpose of harnessing the interconnectivity, and interoperability of responses for remedy, and equipping scope of at least one of the organs, and/or cells such as stem cells, and/or myeloid precursor, and/or platelets, and/or eosinophil, and/or neutrophil, and/or basophil, and/or basophilic type of cells, and/or mast cells, and/or macrophage, and/or plasma cell, and/or sytotoxic t cell, and/or suppressor t cell, and/or helper t cell, and/or lymphoid precursor, and/or b cell, and/or monocyte, and/or tonsils and adenoids, and/or upper lymph nodes, and/or appendix, and/or bone marrow, and/or thymus, and/or spleen, and/or peyer's patches, lower lymph nodes, and/or lymphatic vassals, and/or brain, and/or nerve system, and/or aerobic system, and/or non-aerobic system and any combination thereof, thus once said biologically or photochemically, or electro-optically enhanced liquids and gasses, water, and/or air is consumed, through electrochemical signaling, recognition, and respective memorized, reaction times and procedures, and strings of the immune system participating in the response, or recognition of traceable substances by the brain is being improved, and wherein each of said elements, organs, or cells of the immune system or combinations is quantized and/or referenced and/or stimulated for smoothing non uniformity in responses, and/or updating mhc type 1, 2, 3 and for increasing vitality, awareness, or sensually stimulating the brain or increasing libido, or effecting the chemistry which underline all human physiological interactions through out each individual point in human, animals, and plants life cycle, and thus providing a beneficial points of reference, or reverence from which the immune system could mount adequate responses decisively, swiftly and in tune to the microcosm of events, and environmental parameters.

By the use, at least one moving, produced, protruded, promoted, primed, priorities, or complimented, enhanced, strengthened, triggered, or produced, transferred, or distributed, equipped, armed, or assign cell or organ of the immune system resources is being activated, or replicate on consumption of biologically enhanced and photo-chemically polished drinking water, or breathing air produced in accordance with the reaction caused utilizing, and referencing, or reverencing, memorizing, or updating and calibrating immune system responses in a decisive manner for maximizing remedy orientated action by the immune system to invaders, and wherein such organs or cells include complement, macrophage, viruses, lymphokines, t-cell, b-cell, antibodies, killer cells, and wherein participating organs in such response to said recognition, or memorizing may include tonsiles, adenoids, lymph nodes, appendix, bone marrow, lymphatic cells and vassals, lymph nodes, peyer's patches spleen thymus, brain, and wherein each intake of the biologically enhanced and photochemical polished volume may update individual mhc type 1, 2, 3, and thus updating their awareness, immune system ability to interact and decisively deal with infection or threatening bacteria, or viruses, or antibodies, or diseases and foreign substances, according to the method.

The use of biologically enhanced drinking liquid or breathing gas or a combination thereof, at least one drop, or breathing measure of biological traceable drinking water, or breathing air, a conduit or chamber for storing or holding, transferring, or diluting, concentrating, or adding to said drop, or breathing measure a predetermined amount of water, or air, liquids, or gasses, provisions a high quality, immune system supplement for enhancing immune system in fish, shrimps, prawns, rice fields, wheat fields, aquaculture, dripping and irrigation for vegetation, and fruits, and for the enhancement of water based animals and plants, or for the enhancing of the human immune system opulent response portfolio after consumption or intake of said animals and plants in foods, beverages, and eating supplements.

The biologically enhanced drinking liquid or breathing gas or a combination thereof according to the present invention is useful for the management and maintenance of bodily immune system resources through drinking, or irrigating vegetables and fruits, or for animals and for empowering human immune system continuum of libido-pranic type vitalizing reaction or remedy events and reify relevant transport and distribution, production and definition-swiftly, should an invasion occurs by a bacterium, virus, pathogens, or other noxious species, and wherein triggered defense reaction by the immune system, immune organs, immune cells is decisive, swift, takes shorter time span, and automatically updates mhc 1, 2, 3 and/or causing at least one replication, production, equipping, empowering, chemically modifying, or adding, or subtracting, or signaling, or recognizing, or any combination thereof of at least one participant in said immune response decisive action, and said participants include stem cells, and/or myeloid precursor, and/or platelets, and/or eosinophil, and/or neutrophil, and/or basophil, and/or basophilic type of cells, and/or mast cells, and/or macrophage, and/or plasma cell, and/or sytotoxic t cell, and/or suppressor t cell, and/or helper t cell, and/or lymphoid precursor, and/or b cell, and/or monocyte, and/or tonsils and adenoids, and/or upper lymph nodes, and/or appendix, and/or bone marrow, and/or thymus, and/or spleen, and/or peyer's patches, lower lymph nodes, and/or lymphatic vassals, and/or brain, or any combination, therein involving at least one actively charged, or distributed, maintained and stored, released and targeted, or selected components, and sub-components for action, and remedy.

The use of biologically enhanced drinking liquid or breathing gas or a combination thereof according to the present invention, establishes trigerring in advance, a predetermined immune action by selecting traces according to the method of the present invention wherein at least one components, organ, or cell's performances thus, improved by consumption by drinking and breathing said biologically and optophysiological enhancments, and wherein traceable marks are recognized promoting the enhancment of said components of the immune system in fish, animals, humans or combination involving stem cells, and/or myeloid precursor, and/or platelets, and/or eosinophil, and/or neutrophil, and/or basophil, and/or basophilic type of cells, and/or mast cells, and/or macrophage, and/or plasma cell, and/or sytotoxic t cell, and/or suppressor t cell, and/or helper t cell, and/or lymphoid precursor, and/or b cell, and/or monocyte, and/or tonsils and adenoids, and/or upper lymph nodes, and/or appendix, and/or bone marrow, and/or thymus, and/or spleen, and/or peyer's patches, lower lymph nodes, and/or lymphatic vassals, and/or brain.

The use of the biologically enhanced products according to the present invention, is immunizingly quenching thirst of consumers while improving their species specific responsive, or decisive, or interactive pre-programmed, or predetermined, preset, or variably adjusted for the purpose of harnessing the interconnectivity, and interoperability of responses for remedy, and equipping scope of at least one of the organs, and/or cells such as stem cells, and/or myeloid precursor, and/or platelets, and/or eosinophil, and/or neutrophil, and/or basophil, and/or basophilic type of cells, and/or mast cells, and/or macrophage, and/or plasma cell, and/or sytotoxic t cell, and/or suppressor t cell, and/or helper t cell, and/or lymphoid precursor, and/or b cell, and/or monocyte, and/or tonsils and adenoids, and/or upper lymph nodes, and/or appendix, and/or bone marrow, and/or thymus, and/or spleen, and/or peyer's patches, lower lymph nodes, and/or lymphatic vassals, and/or brain, and/or nerve system, and/or aerobic system, and/or non-aerobic system. the use is beneficial for the management of bodily resources and empowering human immune system by when intake by consume occurs (post 1-8/a-z) prompting the rehearsing of end users major histocompatibility complex or acquired immune system opulent portfolio of defense redemption or remedy, especially beneficial in drinks and foods stuffs, forming action ability to react positively and decisively against invaders of a predetermined space over a predetermined period of time.

In order to implement a facile use of the enhanced products, a bottle or any other appropriate container is holding a globally prepared, and/or specifically produced opto-physiological, photochemically polished drinking water, and said water is held, or stored, temporarily, or permanently, or prominently until consumption for the purpose of quenching thirst, and improving the operation integrity of the Human Immune System (HIS), and wherein at least one organ, and/or cell of the immune system is being effected and improved post consunption, and wherein such immune components may include at least enhanced and accelerated production of immune system elements and/or cells, and or organs, and which a responsive, or decisive, or interactive pre-programmed, or predetermined, preset, or variably adjusted for the purpose of harnessing the interconnectivity, and interoperability of responses for remedy, and equipping scope of at least one of the organs, and/or cells such as stem cells, and/or myeloid precursor, and/or platelets, and/or eosinophil, and/or neutrophil, and/or basophil, and/or basophilic type of cells, and/or mast cells, and/or macrophage, and/or plasma cell, and/or sytotoxic t cell, and/or suppressor t cell, and/or helper t cell, and/or lymphoid precursor, and/or b cell, and/or monocyte, and/or tonsils and adenoids, and/or upper lymph nodes, and/or appendix, and/or bone marrow, and/or thymus, and/or spleen, and/or peyer's patches, lower lymph nodes, and/or lymphatic vassals, and/or brain, and/or nerve system, and/or aerobic system, and/or non-aerobic system and any combination thereof, thus once said biologically or photochemically, or electro-optically enhanced liquids and gasses, water, and/or air is consumed, through electrochemical signaling, recognition, and respective memorized, reaction times and procedures, and strings of the immune system participating in the response, or recognition of traceable substances by the brain is being improved, and wherein each of said elements, organs, or cells of the immune system or combinations is quantized and/or referenced and/or stimulated for smoothing non uniformity in responses, and/or updating mhc type 1, 2, 3 and for increasing vitality, awareness, or sensually stimulating the brain or increasing libido, or effecting the chemistry which underline all human physiological interactions through out each individual point in human, animals, and plants life cycle.

The present invention discloses: “A method for production of a unique Polly-bio-compound (opto-physiologically enhanced, and electro-optically and photochemically polished) forming water based drinks and drips, and/or vapors and or gas, for drinking and irrigation, or combinations for Human, and animal consumption, or for vegetation and fruits, or grains, or nuts, or any combinations, further more, said enhancements could be stored (given for drinking) in said hosts (plants, trees, vegetation, fruits, nuts, grains, sea weeds, or combinations), which stored said enhancements without deteriorating their respective traceable enhancements, or remains, or membrane parts, or proteins, or photochemical damage product portfolio, and more specifically, said portfolio is cyclically, recurrently, non recurrently, or continuously get harvested as said host get ripe, through out the cyclic seasons, said biologically enhanced, traced water, and have through out photosynthesis promote itself for fruition while maintain the original characteristics, shapes of, or geometry, or chemistry seam and physiological theme or string of the originally produced bio-compound (according to the method of the present invention), such Biowater (BW), held or added, stored or released with air, or mixed with flavors or tinted for consumption by and/or through and/or pre/post devices for use according to the methodology of the present invention comprising” an Electro-Triatmic Opto-Physiological Modulator (OPM, ETOPM), especially beneficial for the production of biologically enhanced, traced or resonated polished water (according to the processes in FIG. 1, 1-8/a-z, FIGS. 2-12, ref. 1-100) having at least one trace of, and/or combination of traces, and/or proteins by products or produce combinations, and/or membranes, or photodamage products and by products, or Photocatalysis by products, or electro-catalysis by products, and/or traces produce phonically, and/or photonically (i.e. by the control and/or modulation of photons and photonic interaction, such as in photochemical interactions) (or by multi-photonic absorption facilitated through use of the method according to the present invention, to induce immunity, and to enhance the operation globally, and/or specifically of an individual immune system actions enhancing their responses, velocity, maintenance, abilities, and references, and/or calibration (standards), or helping an individual immune system to deal with potential threats, incoming infectious events, and better prepare an individual immune system to decisively win over the diversity of conditions, which otherwise had to be learned, and dealt with more slowly, and painfully i.e. such as influenza, colds, flu, allergies, bacteria, viruses, pathogens, cancer, transplantation procedures and preparations, medical preparations, foods staffs, drinks such mineral waters, spring water, flavored beverages, and a combinations enhanced with vitamins, or nutrients, or combinations including vegetation's, fruits, nuts, grains, trees, powders and combinations and mixes thereof. A further environmentally preferred embodiment of the present invention is to serve, or irrigate rice while growing for the purpose of storing said optophysiological enhancements therein, and upon harvest and subsequent consumption take effect (i.e. enhancements of the drinking water the rice have thus “consumed”, have in 2^{nd} generation interaction causes enhancements to the immune system of the person who consumed the rice. More specifically, this 1^{st}, and/or, 2^{nd}, and/or 3^{rd} generation interactions (such as humans eating or consuming fruits, nuts, grains, rice, vegetables, seaweed, mushrooms, or drinking mineral waters, waters, spring waters, sprayed waters)

A method for the production of biologically enhanced, enable and potent drinking water having biological material traces and morpheme of protein or representation of DNA and/or RNA signatures, or past replication sequences, and/or repair or reactivation or photodamage portfolio of components, and wherein said representation has been physiological and electro-optically dissociated, or electronically or physically sorted, or by membrane or centrifuge separated or mixed, or reduced, or diluted or modulated and prepared for consumption, once consumed and recognized said biologically able bio-compound is now especially useful for enhancing the Human Immune System.

A method for the production of biologically enhanced, enabled, encrypted, and uncorrupted pure polished potent drinking water

comprising:

Receiving a global water, or liquids, or gasses, or any combination input having a time stamp, containing a known source of biological origin, (FIG. 1, 1-8/a-z, FIGS. 2-12, ref. 1-100) having a predetermined volume and potential for accommodating biomass expansion, and wherein said water, or liquids, or gasses, prior to reaching input phase selector has been cooled, heated, compressed or combination for conversion and adapting to be selected, or bypassed to be processed individually, and wherein said water, and/or liquids, and/or gasses is, or are having a predetermined transmission, or turbidity, or a predetermined level of suspended solids or TSS and/or biological or non biological substances or compounds of species specific proteins or calibration standards with predetermined photo-damage or dissociation resonativistic potential

Accommodating or pumping a predetermined feed, or biomass suspension or expansion or a source of liquids or gasses or solids or any combinations into or through a predetermined conduit or chamber or module or biological settler having a predetermined volume, cross section and a geometry with at least one input and output, or inlet or outlet or combinations of terminals or integrated interfaces for interfacing, or interconnecting, or inter-operating with a control diagnostic router, and/or multi input and output expander with option for onboard concentrators or cylindrical and multi shapes reactors,

Transferring said liquids, and/or gasses, and/or solids or any combination thereof through (FIG. 1, 1-8/a-z, FIGS. 2-12, ref. 1-100) compressing or gating, or by passing or intermodulating said liquids or gases or solids or any combination thereof through a semi opaque pre-filtration unit having a predetermined cut of or threshold or at least one membrane from about 0.1 Micron to about 1000 Micron, a transmission capacity and cut-of threshold for the removal of large particulate materials;

Exposing said liquids, or gases or solids or any combination thereof for bio-expansion (FIG. 1, 1-8/a-z, FIGS. 2-12, ref. 1-100), and opto-physiological, and opto-acousto enhancements by at least one pulsed wave or continuous wave, or transient type of light or sound energies or combination dose having a predetermined fluence rate, average energy, and predetermined intensity, as well as energy density, or combinations from at least one radiation unit interconnected, and/or inter-operated with or interfaced to the multi input and output expander, or control and diagnostic router to at least one multi input, and/or output expander interface, and/or linked to control diagnostic router via electronic means, or optical means (using photonic band gapped fibers, or conventional TIR orientated fibers, or directly delivering, or activating light sources) and/or directly having a high intensity light source therein (i.e. for example: within a module, chamber, conduits, band gapped aerobic wave guide, or combination and wherein at least one wavelength from the region from about 40 nm to about 2400 nm and energy from about 1 micro watt to about a few giga-wafts interferes, or being applied, or geometrically utilized and wherein at least one beam of light from the said radiation unit is stirred or directed for distribution by at least one optical fibers or photonic crystals and/or photonic band gaped platforms (FIGS. 1-11, ref. 1-100) aerobic waveguides or combinations or directly to said liquids, or gases or chamber or conduit or module or any combination thereof or said liquids or gasses, or light, or combination may flow throughout the said guides or directly be compressed or freely flow by gravitation or atmospheric pressure and/or pumping; enhancing and strengthening and expanding repair activity, proteins production, and/or definition yields, and/or by acousto-optic, or sonic, and/or photo-reactivation of the noxious microorganisms contained within said liquids and gasses or any combination over a predetermined period of time from about 1 Millisecond to about 24 hours.

Inactivating said liquids, or gases or solids or any combination thereof by delivering directly, and/or by transferring by photonic band gapped, or conventional high grade fused silica (HGFS), or quartz, or sapphire, or polymer, or organo-elastomers or combination of optical fibers, or non toxic aerobic liquid light guides, at least one pulsed wave, and/or continuous wave, and/or transient type of light or sound, and/or sonic energies or combination dose having a predetermined fluence rate, average energy, and predetermined intensity, as well as energy density, or combinations from at least one additional radiation unit interconnected or inter-operated, or interfaced to said multi input, output expander interface, and/or linked to said control and diagnostic router (CDR), DNA and/or RNA replication sequences of specific noxious species by electro-photochemical, and/or electro-optical, and/or electro optocatalytic processes (FIG. 1, 1-8/a-z, FIGS. 2-12, ref. 1-100), and/or Non thermal plasmas, and/or Electron-beam, and/or Y-rays, and/or Supercritical water Oxidation, and/or Electrohydraulic Cavitation & Sonolysis, and/or Photocatalytic Redox Processes, and/or H2O2/UV, and/or H2O2/O3, and/or O3/UV, variant oxidation agent and/or any combinations thereof (FIG. 1, 1-8/a-z, FIGS. 2-12, ref. 1-100);

Dissociating said liquids, and/or gasses, and/or solids or combinations, optically in optical dissociation module (FIG. 1, 1-8/a-z, FIGS. 2-12, ref. 1-100) by delivering directly, and/or by transferring by photonic band gapped, or conventional high grade fused silica (HGFS), or quartz, or sapphire, or polymer, or organo-elastomers or combination of optical fibers, or non toxic aerobic liquid light guides, at least one pulsed wave, and/or continuous wave, and/or transient type of light or sound, and/or sonic energies or combination dose having a predetermined fluence rate, average energy, and predetermined intensity, as well as energy density, or combinations from at least one additional radiation unit interconnected or inter-operated, or interfaced to said multi input, output expander interface, and/or linked to said control and diagnostic router (CDR), and/or mechanically, electronically, electrooptically, and/or acoustically and/or sonically or combination (FIGS. 1-12, ref. 1-100) for physical separation of said noxious species predetermined components, to form a predetermined volume of Biocompound containing a plurality of predetermined traceable proteins, or photo-damage products;

Transferring (FIG. 1, 1-8/a-z, FIGS. 2-12, ref. 1-100), or phase converting and routing said bio-compound enhancements through a predetermined semi transparent or opaque, post filtration unit or module, or membrane or combination having a cut off from about 0.1 Micron to about 1000 Micron, or by-passing, compounding or mixing or converting said polybiological compound into the gas state, liquid and/or solid state platforms (FIGS. 1-11, ref. 1-100), and/or adjusting its relative temperature up to steam or gradually, or quickly down through condensation to about ice forming, or heating, cooling or compressing, or for combinations thus making a crystalline for morphemic choice of lattice hosting, or tactically aurora aromatically tracing or calibrating continually or non recurrently said bio-able compound to its biological time referenced or reverenced source of origin, or time stamp, thus each individually made volume of quantified bio able compound or any combination thereof may be processed prior to processing by modules, or spheres of the present invention, by variety of cross platform processes selected from electro-photochemical, and/or electro-optical, and/or electro optocatalytic processes, and/or Non thermal plasmas, and/or Electron-beam, and/or Y-rays, and/or Supercritical water Oxidation, and/or Electrohydraulic Cavitation & Sonolysis, and/or Photocatalytic Redox Processes, and/or H2O2/UV, and/or H2O2O3, and/or O3/UV, and/or any combinations thereof in proportions, is apportioned platforms (FIGS. 1-11, ref. 1-100) to represent an elemental of components, which when traced back in time for a period sufficiently long from about 2 to 18 DNA or RNA replication sequences back, for the purpose of furnishing synchronously said quenching bio able compound for human and animal consumption, by drinking and dripping or by irrigating and or temporarily or permanently, or prominently said compound is being held or distributed for storage and bottling, analysis or for feeding animals, or watering agricultural produce to be later consumed by man updating and enhancing consumer's immune system performance within a predetermined period of time.

As an example, a preferred embodiment according to the methodology of the present invention wherein at least one vitamin or nutrient or additional complementing, or harmonious, biocompatible or geneto-compatible substrate or combinations is added to the biologically enhanced drinking water platforms (FIGS. 1-11, ref. 1-100) having a predetermined volume or quantity, according to the method of the present invention platforms (FIGS. 1-11, ref. 1-100) and wherein said addition is aiding, or positively enhancing the carrying, dispensing, release or time release, or distribution in an individual consumer, or producer body improving the inter-operability of the brain, and bon marrow, or organs of the immune system involved in the production and maintenance control and management of the immune system opulent resources such as stem cells, myeloid precursor, platelets, eosinophil, neutrophil, basophilic, basophilic or mast cells, macrophage, plasma cell, sytotoxic t cell, suppressor t cell, helper t cell, lymphoid precursor, b cell, monocyte or any combination thereof, thus enhancing the ability of the human immune system to respond decisively and conserve its resources while, already familiarized threats or invaders, or antigens, or combination thereof are dealt with swiftly platforms (FIGS. 1-11, ref. 1-100) and correctly for maximum remedy effects. A further preferred embodiment of the present invention is especially beneficial for agricultural produce according to the method of the present invention. More specifically, the biologically enhanced, or traceable (i.e. by the immune system) drinking water produced according to the method disclosed by the present invention may be provided for irrigation, dripping, and watering applications of fields, green houses, and special growing houses wherein it is furnished to plants for storage, or for further integrated processing, a further preferred embodiment according to the method of the present invention is wherein said biologically enhanced, and photochemical polished, traceable drinking water is furnished to animals for the production of biologically enhanced foods, such as aquaculture for shrimps or fisheries, prawns and crabs, medical preparations, medical transplants, and other immune orientated compounds and substances. More specifically when said enhanced water is provided for plants having large capacity to accommodate water or contained high percentages of water therein, platforms (FIGS. 1-11, ref. 1-100) such as water melon, lettuce, apples, whet, corn, grains, greens vegetation, milk products, blood products, plasma products. A preferred embodiment according to the present invention will include a pond wherein its water are made according to the processes disclosed in the method of the present invention, and wherein at least one fish, and/or shrimps, and/or prawns, and of crabs, and/or shellfish, and/or sea weeds, and/or sea vegetation may be strengthening their immune system, or soaking in and holding or storing, storing temporarily, or permanently platforms (FIGS. 1-11, ref. 1-100) (permanently means for the duration of their cyclic life hence they may be consumed by human at a later date, (such as in the example of fish, meats, and vegetation and greens, ruits, nuts, and any plants, herbs bushes and trees, and any combination thereof. enhanced as the opulent environment in which they exist and grow in. Claim. 51, A device according to the method of the present invention wherein the module, sphere, conduit, or chamber is embedded, integrated, or attached directly or receiving by photonic band gapped, or directly being projected with, illuminated, irradiated, or exposed to electro magnetic radiation from which at least one photon, or beam of light may include wavelength from about 40 nm, to about 2,400 nm, and wherein the spatial characteristics of the delivered light is uqulized or distributed spatially, or uniformly forming appropriate high energy density zones from about 1 mj per square centimiter, to about 100 Jouls per squers centimeter, and wherein said light is having a pulsed, or continuos characteristics, and wherein each portion of energy directed at target within said modules, or conduits, or chambers is having time domain definitions of intensity, or fluence rate, or fluence dose, or dose, or peak powers from about 0.10 kilowatt, to about 100 Gigawatts, and wherein said light is hybridly generated or singularly generated from at least one laser, or lamp, or flash lamp, or low, high, medium pressure light sources and flasshes, or any combination thereof and wherein said time domain effecting pulse width or pulse duration, or continues average energy delivered at color temperature from 100 kelvin to about 10 thousand kelvin and wherein whitelight, or Ultra violet light, or infrared light is involved in producing said traceable enhancments which upon consumption positivelly enhance the immune system which a responsive, or decisive, or interactive pre-programmed, or predetermined, preset, or variably adjusted for the purpose of harnessing the interconnectivity, and interoperability platforms (FIGS. 1-11, ref. 1-100) of responses for remedy, and equipping scope of at least one of the organs, and/or cells such as stem cells, and/or myeloid precursor, and/or platelets, and/or eosinophil, and/or neutrophil, and/or basophil, and/or basophilic type of cells, and/or mast cells, and/or macrophage, and/or plasma cell, and/or sytotoxic t cell, and/or suppressor t cell, and/or helper t cell, and/or lymphoid precursor, and/or b cell, and/or monocyte, and/or tonsils and adenoids, and/or upper lymph nodes, and/or appendix, and/or bone marrow, and/or thymus, and/or spleen, and/or peyer's patches, lower lymph nodes, and/or lymphatic vassals, and/or brain, and/or nerve system, and/or aerobic system, and/or non-aerobic system.

A preferred embodiment of the present invention wherein using the method of the present invention, (according to claims 1-52) of the present invention wherein at least one module, spheres, conduits or chambers multi input expander, control and diagnostic router, or processing sequence, or dose, photonic, biologic, electronic, catalytic, electrooptics polishing are involved, integrated, or attached, stationary or mobilized by air, sea, and/or on land, by wheels, and floating in a boat, or lifted by transport plains, or hover-crafts, or tracks, and container tracks, and thus once positioned temporarily, or permenetly, or mobilized to disaster areas, or in countries, or geographic location wherein public health and immune systems exhibit deficiencies, or may face threats of infectious incoming events, thus furnishing said bags, or bottles, or cardboard boxes containing said enhancements in water based products especially beneficial for the mobile production plants of biologically enhanced, and opto-physiologically polished drinking bags, preventing recontamination by end users, and updating, and enhancing, strengthening and positively effecting, as well as safely and immunifyinglly quenching thirst of consumers while improving their species specific responsive, or decisive, or interactive pre-programmed, or predetermined, preset, or variably adjusted for the purpose of harnessing the interconnectivity, and interoperability of responses for remedy, and equipping scope of at least one of the organs, and/or cells such as stem cells, and/or myeloid precursor, and/or platelets, and/or eosinophil, and/or neutrophil, and/or basophil, and/or basophilic type of cells, and/or mast cells, and/or macrophage, and/or plasma cell, and/or sytotoxic t cell, and/or suppressor t cell, and/or helper t cell, and/or lymphoid precursor, and/or b cell, and/or monocyte, and/or tonsils and adenoids, and/or upper lymph nodes, and/or appendix, and/or bone marrow, and/or thymus, and/or spleen, and/or peyer's patches, lower lymph nodes, and/or lymphatic vassals, and/or brain, and/or nerve system, and/or aerobic system, and/or non-aerobic system, thus updating populstion s immune systems in geographically seperated locations, as well as providing a valuable enviornmental protection tool, especially beneficial when positioned on mobile platforms (FIGS. 1-11, ref. 1-100) or track, or shipd, or plains, or stationarry positioned in manuifecturing sites, or self sufficiently producing enhancments according to the present invention, emhancing the immune systems of consumers upon consumption (i.e. especially beneficial for the production of biowater, or drinking air).

A preferred embodiment of the present invention wherein using the method of the prsent invention the processing modules and/or spheres are located, positioned, or integral to a track, helicopter, ship, or plain for helping disaster areas wherein natural effects have destroyed infrastructure, and wherein there's luck in clean immunifying drinks, and population are in need of updating or synchronizing their immune system, or MHCs.

A preferred embodiment of the present invention wherein a novel application platforms, are presented with associated processes for environmental protection of public health.

Examples of the applications therein does not intend to limit the scope of the present invention what so ever; according to claims of the method of the present invention; (FIGS., 1-100,(FIGS. 2-12, ref. 1-70,), (research disclosure, abstract, and experimental data attached for clarity and to portray, and fortify the economic benefits and advantages associated with the present invention; “A ship in a bottle synthesis of Biowater production processes” is herewith disclosed:

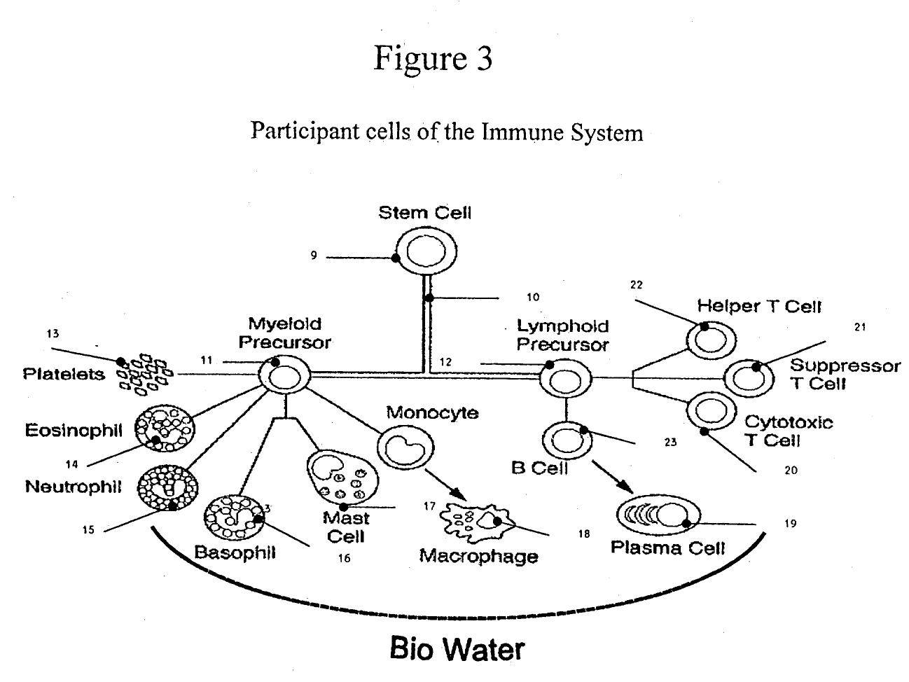

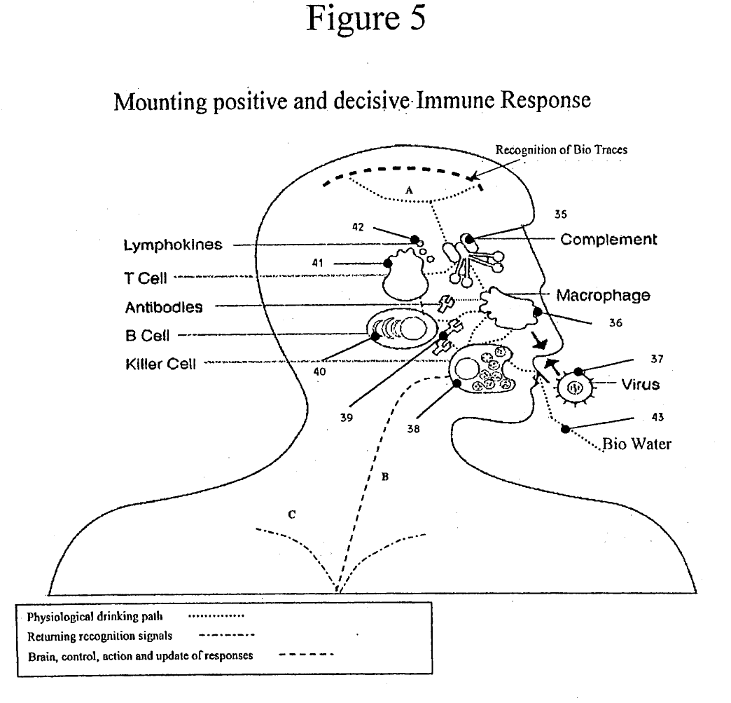

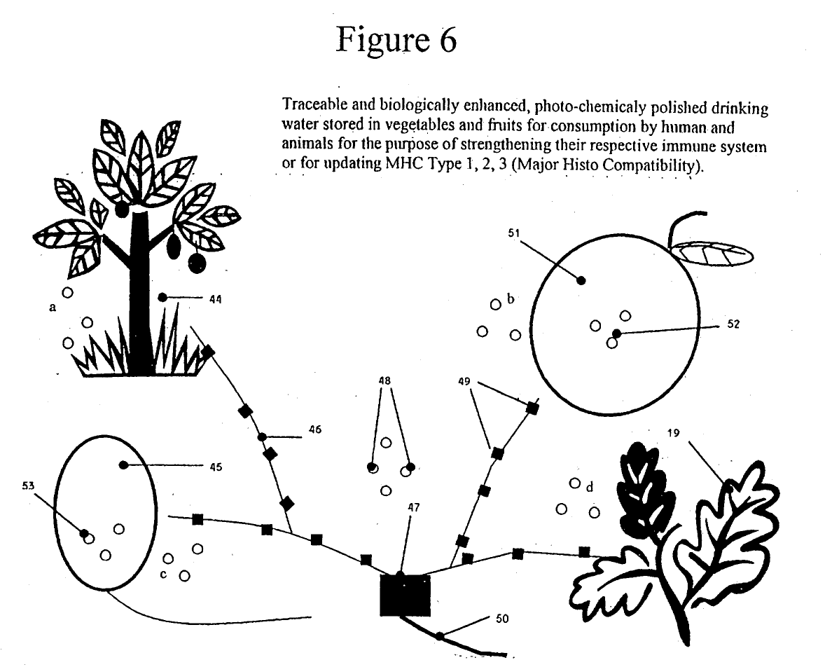

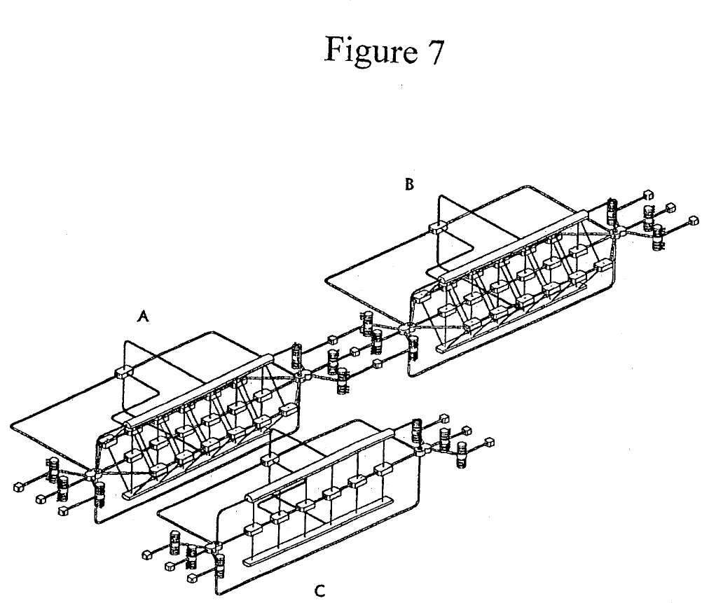

“A triad Spheroid Electro-Triatmic opto-electronic, Acousto-optic modulator Drinking water and breathing air Compound, or liquids and gasses formed by Opto-electronic opto-physiological method {(as in FIG. 1, 1-8, FIG. 1, 1-8a-z), FIG. 3, references 9-23, FIG. 4, 24-34, FIG. 5 35-43, FIG. 6, FIG. 7) or (FIGS. 1-8/a-z) for production of Biologically enhanced traceable, electro-optically polished drinking water, and breathing air, or liquids or gasses, especially beneficial for the management and maintenance of bodily resources through drinking, or irrigating, and empowering Human immune system continuum of libido-pranic type vitalizing reaction or remedy events and reify relevant transport and distribution, production and definition-swiftly, should an invasion occurs by a bacterium, virus, pathogens, or other noxious species.

More specifically in order to portray the novelty of the present invention, the following figures from 1-12 and from 7-12, (FIGS. 1-12, ref. 1-100) illustrate the processes according to the present invention and does not intend to limit the scope of the invention in any way what so ever.

Figure one illustrate a schematic view of the processes according to the present invention especially beneficial for the production of biologically enhanced and polished drinking water the. A method for the production of Biologically enhanced and polished water (i.e. Biowater) is forthwith presented, comprising:

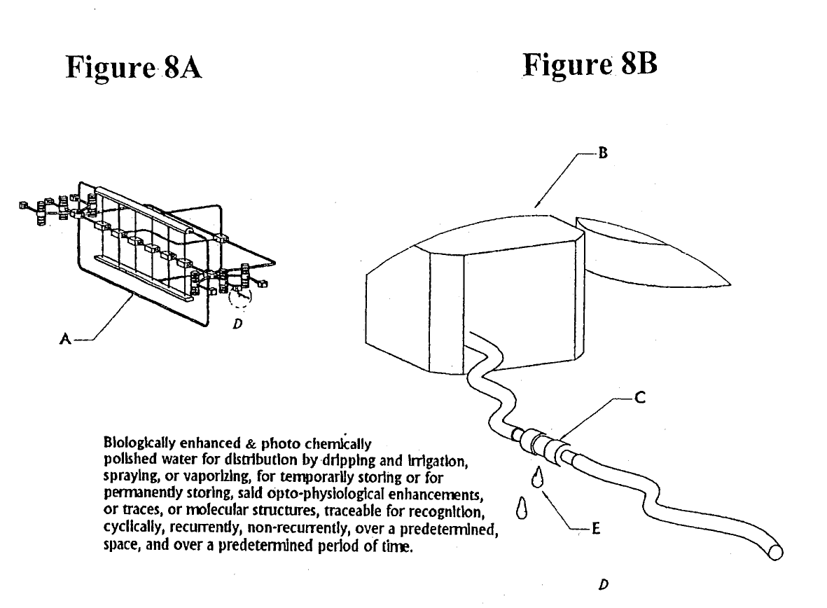

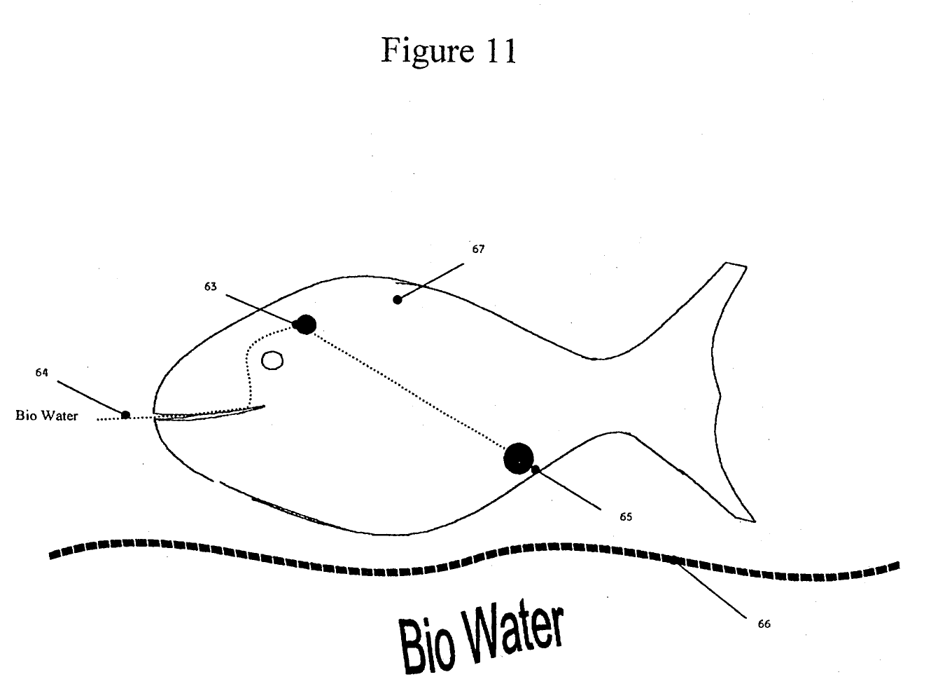

FIG. 1, (1-8), (a-z), A method for production of biological enhanced or traced or photo-chemically polished drinking water, comprising: Water or water based liquids or gasses or mixes are being selected (1) at the input to at least one phase selector (2);Transmitting said liquids or gasses or combination through at least one pre-filtration unit or interface prior to bio-mass expansion module at a flow rate from about 1mililiter/hour to about 700,000 Litters/hours or triplicate if better transmission and thousand fold higher when synchronously operated for maximizing production yields of polybiological water, and wherein said biologically enhanced water (Biowater) is transgressing, or trance-verging, or transversing at least one conduit, or chamber (1), (7), (FIGS. 1-8, 2-12), (a-z) module, (7, 6, 5,) further inactivating, dissociating, and/or sorting mechanically, or optically, or recurrently, or non-recurrently being transformed and polished, enhanced and bio-traced, recycled or pass through a single or multi-passed to become, health promoting, and reducing the stress, calibrating, rehearsing, and educating human immune system (HIS), its vast portfolio of positive reaction and decisive econo-physiological Interconnectivity solution (i.e. body post consumption is using its own resources conservatively and in tune with its buffers or raw potentials for defense reaction against invader and/or inter-operability between the brain of an individual consume to its bon marrow production ability of immunoglobulines, or electrochemically through photochemistry for 1^{st}, 2^{nd}, or 3^{rd}, generation consumption interaction of at least one protein accumulated or volume compounded (FIG. 1, 1-8) will be, especially beneficial for domestic, commercial, agricultural, environmental and medical applications wherein water or air (i.e. liquids and/or gasses) is used as hosting media from which, hoses could traduces, or tract, or traduce, or induce proceeding to accommodate or accomodantic compound wide variety of useful applications such as found in mineral water production and bottling and production facilities, or aquifers (FIG. 1, 1-8/a-z), or factories (FIG. 1, 1-8/a-z), spring water production sites (1-8, a-z), Disinfection sites for drinking water or wastewater (1-8), (a-z), filtration sites (FIG. 1, 1-8/a-z), and generally any water treatment site (FIG. 1, 1-8, a-z) which may provide water for drinking. Any order of modules, conduits, or chambers may be involved in processes according to the present invention such as pipes and tanks, chambers, and/or containers and photonic band gaped waveguides (FIGS. 11-8/a-z), aerobic and/or non toxic guides for liquids and gasses, or combinations or liquid waveguides, HGFS (1-8/a-z, including multi inputs and outputs expander interface (MIOEI) and other conventional TIR type waveguides from glass and polymers, any and all of the involved Modules, conduits and chambers could be interface (FIG. 1, 1-8/a-z) in any order (1-8, a-z FIG. 1 of the present invention)including the use of photonic band gaped waveguides to serve as reactors and/or modules for the purpose of implementing and assimilating effecting and operating devices according to the methodology of the present invention, the method of the present invention include the sequential, cyclic, current, recurrent, non-recurrent, turbulent and uniform flow types involving at least one components selected by the input selector which is having any number of input selection inserts therein (only 8 shown at input and output) and distributed for processing by and through input phase selector (1), pre-filtration (2), biomass expansion module (3), inactivation module (4), optical dissociation and/or particle size reduction effects are applied, prior or after physiological or mechanical physical separation and sorting module, phase converter, and router and wherein a multi I/Os Expander Module is illustrated intentionally for clarity, showing and providing Interconnectivity and interoperability between any connected or linked synchronized or aligned or interfaced conduits, chambers, modules and so facilitate a wide diversity of geometrical utilization of devices using the method of the present invention in conjunction with, or independently, or directly receiving additional (direct) input or involvement and support for electro-photochemical, and/or electro-optical, and/or electro optocatalytic processes, and/or Non thermal plasmas, and/or Electron-beam, and/or Y-rays, and/or Supercritical water Oxidation, and/or Electrohydraulic Cavitation & Sonolysis, and/or Photocatalytic Redox Processes, and/or H2O2/UV, and/or H2O2O3, and/or O3/UV, and/or any combinations thereof, an external processing module (EPM) is illustrated connected, and linked (B) and is shown for clarity externally connected (B), for expanding processing or saving time when reactivating, or biomass expanding, or inactivating, or strengthening, or selecting or routing, or phase converting or processing according to the method of the present invention, when liquids and gases are being processed therein (within the processes, stages, phases, spheres) such as drinking water, gasses, solids having a predetermined size, volume or predetermined quantities said processing is involving magnetic radiation, electro-magnetic radiation, light sources (any type of light source or combination covering both CW, or PW, Continuous wave types, and/or Pulsed Wave types) to include lamps, lasers, flash-lamps, FELS, EAFELs, or hybrid, or synchronized multi tasking and multi processing, or any processing elements using simultaneous or separate, sequential or polyphonic, or multiphotonic absorption processes, or diagnostics by dissorption for operation of light source and energy sources in the flow chart and schematic illustration of the method according to the present invention, (F) represent a manifold input, and/or output accessible to each or any of the (reactors), modules, or conduits, or chambers, and wherein external feeds could be routed directly to any of the processing modules according to the method of the present invention allowing end users and producers to configure the system to their own individual needs (A) represent a bypass allowing directly routing any liquid or gas to be processed and connecting or interfacing input or output to the External Processing Module (EPM), (B) represent the connection between the EPM, and the CDR (Control Diagnostic Router), (C) represent a passage, or flow through conduit from the external Processing module (EPM), (D) represent an identical transfer conduit as ((B)) and is designated for connection of the multi I/Os interface for the enhancement of command control and Interconnectivity, and interoperability between the expander and the external processing module (EPM), (E) represent a link between CDR, and input phase selector and the phase converter router (FIG. 1, 1-8/a-z), (G) represent a global by pass allowing direct transmission, or delivery, or flow through of any substance to be processed according to the method of the present invention, without actually being processed by any of the processing modules of the present invention, especially beneficial for the production of biologically and photo-chemically enhanced and polished liquids and gasses wherein diluting or concentration, addition or complementation is desired according to the method of the present invention, or as a preparatory stage or phase, or sphere preparing for entry or input into the EPM (External Processing Module) module Compression, cooling, and heating are included as preprocessing elements for the provision of the different state desired in the processing according to the method of the present invention, these pre processing are included for clarity illustrating the ability of the present invention to change, or alter the phase (i.e. for an example: such as water, steam, and back to water, or from water to ice, and through the condensation temperature to water again). (A, B, C, D, E, F, G) thus method for the production of biologically enhanced drinking water and/or liquids and gasses, or solids or combination is herewith disclosed, enable, encrypted, and made uncorruptedlly pure, by photo-chemically polished potently for immunifying, impressed with substrate representations and apportioned comprising:

Receiving a global water, or liquids, or gasses, or any combination input having a time stamp, containing a known source of biological origin, (FIG. 1, 1-8/a-z, FIGS. 2-12, ref. 1-100) having a predetermined volume and potential for accommodating biomass expansion, and wherein said water, or liquids, or gasses, prior to reaching input phase selector has been cooled, heated, compressed or combination for conversion and adapting to be selected, or bypassed to be processed individually, and wherein said water, and/or liquids, and/or gasses is, or are having a predetermined transmission, or turbidity, or a predetermined level of suspended solids or TSS and/or biological or non biological substances or compounds of species specific proteins or calibration standards with predetermined photo-damage or dissociation resonativistic potential

Accommodating or pumping a predetermined feed, or biomass suspension or expansion or a source of liquids or gasses or solids or any combinations into or through a predetermined conduit or chamber or module or biological settler having a predetermined volume, cross section and a geometry with at least one input and output, or inlet or outlet or combinations of terminals or integrated interfaces for interfacing, or interconnecting, or-inter-operating with a control diagnostic router, and/or multi input and output expander with option for onboard concentrators or cylindrical and multi shapes reactors;

Transferring said liquids, and/or gasses, and/or solids or any combination thereof through (FIG. 1, 1-8/a-z, FIGS. 2-12, ref. 1-100) compressing or gating, or by passing or inter-modulating said liquids or gases or solids or any combination thereof through a semi opaque pre-filtration unit having a predetermined cut of or threshold or at least one membrane from about 0.1 Micron to about 1000 Micron, a transmission capacity and cut-of threshold for the removal of large particulate materials;

Exposing said liquids, or gases or solids or any combination thereof for bio-expansion (FIG. 1, 1-8/a-z, FIGS. 2-12, ref. 1-100), and opto-physiological, and opto-acousto enhancements by at least one pulsed wave or continuous wave, or transient type of light or sound energies or combination dose having a predetermined fluence rate, average energy, and predetermined intensity, as well as energy density, or combinations from at least one radiation unit interconnected, and/or inter-operated to at least one multi input, and/or output expander interface, and/or linked to control diagnostic router, and/or directly having a high intensity light source therein (i.e. for example: within a module, chamber, conduits, band gapped aerobic wave guide, or combination and wherein at least one wavelength from the region from about 40 nm to about 2400 nm and energy from about 1 micro watt to about a few giga-wafts interferes, or being applied, or geometrically utilized and wherein at least one beam of light from the said radiation unit is stirred or directed for distribution by at least one optical fibers or photonic crystals and/or photonic band gaped aerobic waveguides or combinations or directly to said liquids, or gases or chamber or conduit or module or any combination thereof or said liquids or gasses, or light, or combination may flow throughout the said guides or directly be compressed or freely flow by gravitation or atmospheric pressure and/or pumping; enhancing and strengthening and expanding repair activity, proteins production, and/or definition yields, and/or by acousto-optic, or sonic, and/or photo-reactivation of the noxious microorganisms contained within said liquids and gasses or any combination over a predetermined period of time from about 1 Millisecond to about 24 hours.

Inactivating said liquids, or gases or solids or any combination thereof by delivering directly, and/or by transferring by photonic band gapped, or conventional high grade fused silica (HGFS), or quartz, or sapphire, or polymer, or organo-elastomers or combination of optical fibers, or non toxic aerobic liquid light guides, at least one pulsed wave, and/or continuous wave, and/or transient type of light or sound, and/or sonic energies or combination dose having a predetermined fluence rate, average energy, and predetermined intensity, as well as energy density, or combinations from at least one additional radiation unit interconnected or inter-operated, or interfaced to said multi input, output expander interface, and/or linked to said control and diagnostic router (CDR),DNA and/or RNA replication sequences of specific noxious species by electro-photochemical, and/or electro-optical, and/or electro optocatalytic processes (FIG. 1, 1-8/a-z, FIGS. 2-12, ref. 1-100), and/or Non thermal plasmas, and/or Electron-beam, and/or Y-rays, and/or Supercritical water Oxidation, and/or Electrohydraulic Cavitation & Sonolysis, and/or Photocatalytic Redox Processes, and/or H2O2/UV, and/or

H2O2/O3, and/or O3/UV, variant oxidation agent and/or any combinations thereof (FIG. 1, 1-8/a-z, FIGS. 2-12, ref. 1-100);

Dissociating said liquids, and/or gasses, and/or solids or combinations, optically in optical dissociation module (FIG. 1, 1-8/a-z, FIGS. 2-12, ref. 1-100) by delivering directly, and/or by transferring by photonic band gapped, or conventional high grade fused silica (HGFS), or quartz, or sapphire, or polymer, or organo-elastomers or combination of optical fibers, or non toxic aerobic liquid light guides, at least one pulsed wave, and/or continuous wave, and/or transient type of light or sound, and/or sonic energies or combination dose having a predetermined fluence rate, average energy, and predetermined intensity, as well as energy density, or combinations from at least one additional radiation unit interconnected or inter-operated, or interfaced to said multi input, output expander interface, and/or linked to said control and diagnostic router (CDR), and/or mechanically, electronically, electro-optically, and/or acoustically and/or sonically or combination (FIGS. 1-12, ref. 1-100) for physical separation of said noxious species predetermined components, to form a predetermined volume of Bio-compound containing a plurality of predetermined traceable proteins, or photo-damage products; Transferring (FIG. 1, 1-8/a-z, FIGS. 2-12, ref. 1-100), or phase converting and routing said bio-compound enhancements through a predetermined semi transparent or opaque, post filtration unit or module, or membrane or combination having a cut off from about 0.1 Micron to about 1000 Micron, or by-passing, compounding or mixing or converting said Polly-biological compound into the gas state, liquid and/or solid state, and/or adjusting its relative temperature up to steam or gradually, or quickly down through condensation to about ice forming, or heating, cooling or compressing, or for combinations thus making a crystalline for morphemic choice of lattice hosting, or tactically aurora aromatically tracing or calibrating continually or non recurrently said bio-able compound to its biological time referenced or reverenced source of origin, or time stamp, thus each individually made volume of quantified bio able compound or any combination thereof may be processed prior to processing by modules, or spheres of the present invention, by variety of cross platform processes selected from electro-photochemical, and/or electro-optical, and/or electro opto-catalytic processes, and/or Non thermal plasmas, and/or Electron-beam, and/or Y-rays, and/or Supercritical water Oxidation, and/or Electrohydraulic Cavitation & Sonolysis, and/or Photocatalytic Redox Processes, and/or H2O2/UV, and/or H2O2O3, and/or O3/UV, and/or any combinations thereof in proportions, is apportioned to represent an elemental of components, which when traced back in time for a period sufficiently long from about 2 to 18 DNA or RNA replication sequences back, for the purpose of furnishing synchronously said quenching bio able compound for human and animal consumption, by drinking and dripping or by irrigating and or temporarily or permanently, or prominently said compound is being held or distributed for storage and bottling, analysis or for feeding animals, or watering agricultural produce to be later consumed by man updating and enhancing consumer's immune system performance within a predetermined period of time.

FIG. 2 illustrate a schematic view of a single sphere of multi-processing especially beneficial for the production of biologically enhanced and photo-chemically polished drinking water the. A method and devices using said method for the production of Biologically enhanced and photo-chemically polished water (i.e. Biowater) is forthwith presented, comprising: A multi-processing Electro-Triatmic Spheroid Modulator (ETSM) workstation (FIG. 1, 1-8/a-z), (FIG. 2, 1-8/a-z), (A, B, C, D, E, F, G), according to the method of the present invention for the production of biologically enhanced, traced or polished drinking water (FIG. 1, 1-8/a-z), (FIG. 2, 1-8/a-z) especially beneficial for automatically or synchronously educating, rehearsing and enhancing all types of Major Histocompatibility Complexes (FIGS. 1-7) (i.e. MHC types: I, II, III,) and upon consumption promote immunifying by predetermined stimulated reactions for the purpose of dynamically activating a continuum of smooth and released from hindrances, and/or miss-conceptual recognition, improves vitality and operational-decisiveness of the relevant immune response reactions. Further more, the consumer by enhancing long term operation performance of the immune system by intro-fusing a series of synthesized Opto-physiological (FIG. 1, 1-8/a-z), (FIG. 2, 1-8/a-z), (A, B, C, D, E, F, G), for consumption a novel Polly-Bio-Compound (PBC) which when consumed to quench thirst, automatically reference or calibrate, or rehearse or updates the Human brain ability to flawlessly update for production, equip and/or remind of remedy or production ability of species specific calibration standards, said polybiocompound post consumption augment or surpass refreshing synchronously the continuum in which an individual own defense mechanism's react and the subjective time resolution underlining the Interconnectivity and interoperability between relevant component of the immune system defenses, their transitive (A, B, C, D, E, F, G), and/or transitive, or translative powerhouse of organs (FIG. 4, 24-34), (FIG. 3, 9-23), each with its own specie specific calibration standards (FIG. 1, 1-8/a-z, FIG. 2, FIG. 3, FIG. 4, FIG. 5) including (a) Cyclic (FIG. 1,a-z), (b) Recurrent (FIG. 1,a-z), (c) Non-Recurrent productive ability for action against invaders, their menu of control commands and programmability for equipping relevant hydrapneumatic Opto-electrotriatmic phase modulation effecting the chemistry for which a particular sphere (FIG. 1, a-z of the present invention) and the field of action within which a predetermined positive decisive immune system-action occurs against a specific bacteria, and/or virus, and/or Pathogen or combinations at high resolution maximizing recognition or utilization of resources (FIG. 3, 4, 5, 6, 7) or combination for the purpose of accelerating the preparation phase (FIG. 1, 1-8/a-z) for effective economical redemption, and/or remadiation thus been remediative (FIG. 3, 9-23), (FIG. 4, 24-34) within a predetermined individual's own body space (FIG. 3, 4, 5, 6, 7) and the immune system operation therein (FIG. 3, 4, 5, 6, 7) or volume over a predetermined period of time for which complete remedy is signaling.

FIG. 3 illustrate a schematic view of the beneficial interaction achieved by consumption of biologically enhanced, photo-chemically polished drinking water and the enhancing effects are forthwith presented comprising: utilizing the method according to the method of the present invention (FIG. 1, 1-8,a-z), (FIG. 2, 9-23, A, B, C, D, E, F, G), a process of command control interaction in the brain (not shown) is effecting many organs of the body (FIG. 3, 9-23) and their respective production or chemical and/or biological, and or physiological(FIG. 3, 9-23) activities involving the participation of a plurality of cells (FIG. 3, 9-23) processes (FIG. 1, 1-8/a-z), (FIG. 2. A, B, C, D, E, F, G), according to the present invention especially beneficial for the production of biologically enhanced and photo-chemically polished drinking water, the. A method for the production of Biologically enhanced and polished water (i.e. Biowater) is forthwith presented, comprising an enhancement, in the operability, capabilities, abilities, and Interconnectivity associated with the interoperability of the cells, their respective immune system organs of the body and thus the benefits or the potential for the specific tuning of the enhancements is illustrated (participants shown), and the photo-chemical interactions (FIG. 1, 1-8/a-z not shown) and polishing could be tuned in a predetermined velocity or dilution or additives, or additions or biomass expansion, or by photochemical inactivation, or dissociation or by electro-catalysis, or photo-catalysis, or sound, or ultrasound (FIG. 1, 1-8/a-z) in such way as to cause updates (FIG. 3, 4, 5, 6, 7) in the immune system and to resolve and link, synchronize and protect and remedy (FIG. 1, 2, 3, 4, 5, 6, 7) public health in accordance with the method of the present invention (FIG. 2, 9-23, A, B, C, D, E, F, G) through the drinking of the biologically enhanced and photo polished Biowater according to the method of the present invention.

FIG. 4, illustrate a method for the production of biologically enhanced liquids according to the method of the present invention for the creation of biologically enhanced liquids and gasses (such as air or water), comprising: traceable for recognition and photo-chemically polished; (in line with the production processes (FIG. 1, 1-8/a-z)) method is forthwith presented which trigger, activates, enhance the operation of, and increase the reference of stimulation and calibration of the participant organ of the immune system (FIG. 4, 24-34) the organs is illustrated in their respective physical location distributed for clarity and their names indicated for clear orientation, these organs (FIG. 4, 24-34) gets activated once consumption of the enhanced liquids and gasses peruse, these organs are partially responsible for the creation manipulation and empowering capabilities to equip the cells of the immune system (such as indicated in FIG. 3, 9-24). Water or air provide a godd hosting mediums for the storage and or temporary hosting or carrying medium to/from consumers body and could also be given or distributed for irrigation or dripping system for vegetation and fruits types having large amounts of water therein (such as water melon, apple, lettuce, mushrooms, not shown in FIG. 4), such agricultural produce could also be consumed by end users or producers and update and enhanced their immune system through foods and beverages (i.e. diet) as well as through drinking said enhanced water which have been produced according to the method of the present invention, the frame shown encapsulating the organs of the immune system is designated for illustrating the scope and the wide positively enhancements, and effects which encapsulate directly or indirectly through triggering, educating, rehearsing and maintaining the operational opulence of the immune system according to the effects caused once consumption of the Biowater according to the present invention peruse.

FIG. 5, illustrates consumption of biologically enhanced and photochemical polished drinking water, in accordance with the method of the present invention. A biologically and photochemical enhanced drinking water is shown being consumed by an individual comprising: Positive effects of the immune system (FIG. 5, 35-43, B, A, C) caused by the consumption of Biowater according to the method of the present invention (FIG. 1, 1-8/a-z, FIG. 2, 3, 4) is illustrated herewith, effects on the brain and Histo-compatibility or MHC type 1, 2, 3 is also included (FIG. 5, 35-43), the biologically enhanced and photo-chemically polished drinking water is shown being consumed (43) and going down on their way out of the consumer body (actual output not shown), (C) represent incoming signals and recognition stemming from within the human body (C) and delivered by electrochemical signals to the brain (not shown), (A) represent the increased awareness caused by the consumption and subsequent recognition of biological origin in the brain, the immune system is illustrated as having a response, or decisive response prompted or triggered by the Biowater, or bioair, or biologically enhanced and photo-plasmic, or photochemical effects of the method of the present invention (FIG. 1, 1-8/a-z, FIG. 2, 3, 4), especially beneficial when consumed in water based compounds (FIG. 5, 35-43), as illustrated by the figures of the present invention all effects are disclosed herein both physical drinking path, returning recognition signals, and the brain effecting, controlling, and updating the response of the immune system, making and causing it to occur in a high resolution and in a decisive manner, the present method of the invention also facilitate the updating of the immune system responses swiftly and without drawing accesive resources from a depleted, or suffering immune system responsiveness.

FIG. 6, illustrate the opto-physiological enhancement of the present invention in fruits and vegetables such as lettuce, water melons, apples, nuts, grains, rice, wheat for bread and flower, said enhancement is distributed in the form of water drips and irrigation, is aimed at illustrating that the consumption of said enhancements could be of fruits, vegetables, and plants (2^{nd} generation consumption interaction) rather then direct consummation of any liquid, or gasses, or combination, especially beneficial for the enhancement of the Human Immune system.

FIG. 7, illustrate an example for serially, or parallel connection (A&B), (A&C), inter-operated, synched, or linked interfaced systems, FIG. 7, illustrate a device orientation utilizing the methodology of the present invention, according to the present invention, a parallel connection is illustrated (A&C), in order to portray the flexibility of the method of the present invention (serially connected for high flow rates, while parallel connected for thoroughness, and high resolution of end results, and enhancements, according to the methodology of the present invention each of the system or combination is capable of performing independently a method for the production of biologically enhanced drinking water and/or liquids and gasses, or solids or combination enable, encrypted, and made uncorruptedlly pure, by photo-chemically polished potently for immunifying, impressed with substrate representations and apportioned comprising:

Receiving a global water, or liquids, or gasses, or any combination input having a time stamp, containing a known source of biological origin, (FIG. 1, 1-8/a-z, FIGS. 2-12, ref. 1-100) having a predetermined volume and potential for accommodating biomass expansion, and wherein said water, or liquids, or gasses, prior to reaching input phase selector has been cooled, heated, compressed or combination for conversion. and adapting to be selected, or bypassed to be processed individually, and wherein said water, and/or liquids, and/or gasses is, or are having a predetermined transmission, or turbidity, or a predetermined level of suspended solids or TSS and/or biological or non biological substances or compounds of species specific proteins or calibration standards with predetermined photo-damage or dissociation resonativistic potential

Accommodating or pumping a predetermined feed, or biomass suspension or expansion or a source of liquids or gasses or solids or any combinations into or through a predetermined conduit or chamber or module or biological settler having a predetermined volume, cross section and a geometry with at least one input and output, or inlet or outlet or combinations of terminals or integrated interfaces for interfacing, or interconnecting, or inter-operating with a control diagnostic router, and/or multi input and output expander with option for onboard concentrators or cylindrical and multi shapes reactors;

Transferring said liquids, and/or gasses, and/or solids or any combination thereof through (FIG. 1, 1-8/a-z, FIGS. 2-12, ref. 1-100) compressing or gating, or by passing or inter-modulating said liquids or gases or solids or any combination thereof through a semi opaque pre-filtration unit having a predetermined cut of or threshold or at least one membrane from about 0.1 Micron to about 1000 Micron, a transmission capacity and cut-of threshold for the removal of large particulate materials;

Exposing said liquids, or gases or solids or any combination thereof for bio-expansion (FIG. 1, 1-8/a-z, FIGS. 2-12, ref. 1-100), and opto-physiological, and opto-acousto enhancements by at least one pulsed wave or continuous wave, or transient type of light or sound energies or combination dose having a predetermined fluence rate, average energy, and predetermined intensity, as well as energy density, or combinations from at least one radiation unit interconnected, and/or inter-operated to at least one multi input, and/or output expander interface, and/or linked to control diagnostic router, and/or directly having a high intensity light source therein (ABC, or any combinations) (i.e. for example: within a module, chamber, conduits, band gapped aerobic wave guide, or combination and wherein at least one wavelength from the region from about 40 nm to about 2400 nm and energy from about 1 micro watt to about a few giga-watts interferes, or being applied, or geometrically utilized and wherein at least one beam of light from the said radiation unit is stirred or directed for distribution by at least one optical fibers or photonic crystals and/or photonic band gaped aerobic waveguides or combinations or directly to said liquids, or gases or chamber or conduit or module or any combination thereof or said liquids or gasses, or light, or combination may flow throughout the said guides or directly be compressed or freely flow by gravitation or atmospheric pressure and/or pumping; enhancing and strengthening and expanding repair activity, proteins production, and/or definition yields, and/or by acousto-optic, or sonic, and/or photo-reactivation of the noxious microorganisms contained within said liquids and gasses or any combination over a predetermined period of time from about 1 Millisecond to about 24 hours (ABC, or any combinations).

Inactivating said liquids, or gases or solids or any combination thereof by delivering directly, and/or by transferring by photonic band gapped, or conventional high grade fused silica (HGFS), or quartz, or sapphire, or polymer, or organo-elastomers or combination of optical fibers, or non toxic aerobic liquid light guides, at least one pulsed wave, and/or continuous wave, and/or transient type of light or sound, and/or sonic energies or combination dose having a predetermined fluence rate, average energy, and predetermined intensity, as well as energy density, or combinations from at least one additional radiation unit interconnected or inter-operated, or interfaced to said multi input, output expander interface, and/or linked to said control and diagnostic router (CDR),DNA and/or RNA replication sequences of specific noxious species by electro-photochemical, and/or electro-optical, and/or electro optocatalytic processes (FIG. 1, 1-8/a-z, FIGS. 2-12, ref. 1-100), and/or Non thermal plasmas, and/or Electron-beam, and/or Y-rays, and/or Supercritical water Oxidation, and/or Electrohydraulic Cavitation & Sonolysis, and/or Photocatalytic Redox Processes, and/or H2O2/UV, and/or H2O2/O3, and/or O3/UV, variant oxidation agent and/or any combinations thereof (FIG. 1, 1-8/a-z, FIGS. 2-12, ref. 1-100);

Dissociating said liquids, and/or gasses, and/or solids or combinations, optically in optical dissociation module (FIG. 1, 1-8/a-z, FIGS. 2-12, ref. 1-100) by delivering directly, and/or by transferring by photonic band gapped, or conventional high grade fused silica (HGFS), or quartz, or sapphire, or polymer, or organo-elastomers or combination of optical fibers, or non toxic aerobic liquid light guides, at least one pulsed wave, and/or continuous wave, and/or transient type of light or sound, and/or sonic energies or combination dose having a predetermined fluence rate, average energy, and predetermined intensity, as well as energy density, or combinations from at least one additional radiation unit interconnected or inter-operated, or interfaced to said multi input, output expander interface, and/or linked to said control and diagnostic router (CDR), and/or mechanically, electronically, electro-optically, and/or acoustically and/or sonically or combination (FIGS. 1-12, ref. 1-100) for physical separation of said noxious species predetermined components, to form a predetermined volume of Bio-compound containing a plurality of predetermined traceable proteins, or photo-damage products;

Transferring (FIG. 1, 1-8/a-z, FIGS. 2-12, ref. 1-100), or phase converting and routing said bio-compound enhancements through a predetermined semi transparent or opaque, post filtration unit or module, or membrane or combination having a cut off from about 0.1 Micron to about 1000 Micron, or by-passing, compounding or mixing or converting said Polly-biological compound into the gas state, liquid and/or solid state, and/or adjusting its relative temperature up to steam or gradually, or quickly down through condensation to about ice forming, or heating, cooling or compressing, or for combinations thus making a crystalline for morphemic choice of lattice hosting, or tactically aurora aromatically tracing or calibrating continually or non recurrently said bio-able compound to its biological time referenced or reverenced source of origin, or time stamp, thus each individually made volume of quantified bio able compound or any combination thereof may be processed prior to processing by modules, or spheres of the present invention, by variety of cross platform processes selected from electro-photochemical, and/or electro-optical, and/or electro opto-catalytic processes, and/or Non thermal plasmas, and/or Electron-beam, and/or Y-rays, and/or Supercritical water Oxidation, and/or Electrohydraulic Cavitation & Sonolysis, and/or Photocatalytic Redox Processes, and/or H2O2/UV, and/or H2O2O3, and/or O3/UV, and/or any combinations thereof in proportions, is apportioned to represent an elemental of components, which when traced back in time for a period sufficiently long from about 2 to 18 DNA or RNA replication sequences back, for the purpose of furnishing synchronously said quenching bio able compound for human and animal consumption, by drinking and dripping or by irrigating and or temporarily or permanently, or prominently said compound is being held or distributed for storage and bottling, analysis or for feeding animals, or watering agricultural produce to be later consumed by man updating and enhancing consumer's immune system performance within a predetermined period of time.

FIG. 8, illustrate a block schematic view of dripping (C) irrigation output from system (FIGS. 1-11), to a dripping pipe (C), drops of the opto-physiologically enhanced water are herewith presented (E), (D) represent the output from the system according to the present invention, while (B) represent an interface capable of cooling, heating, and/or compression for accommodating wide varieties of end users end connection equipment for the distribution of such enhancements according to the methodology of the present invention. Fruits, and/or vegetation, or plants, animals may be consumed (not shown) after dripping, and/or watering have occur, and so benefits gets transferred to said veg, fruits, plants, animals for later consumption by humans.

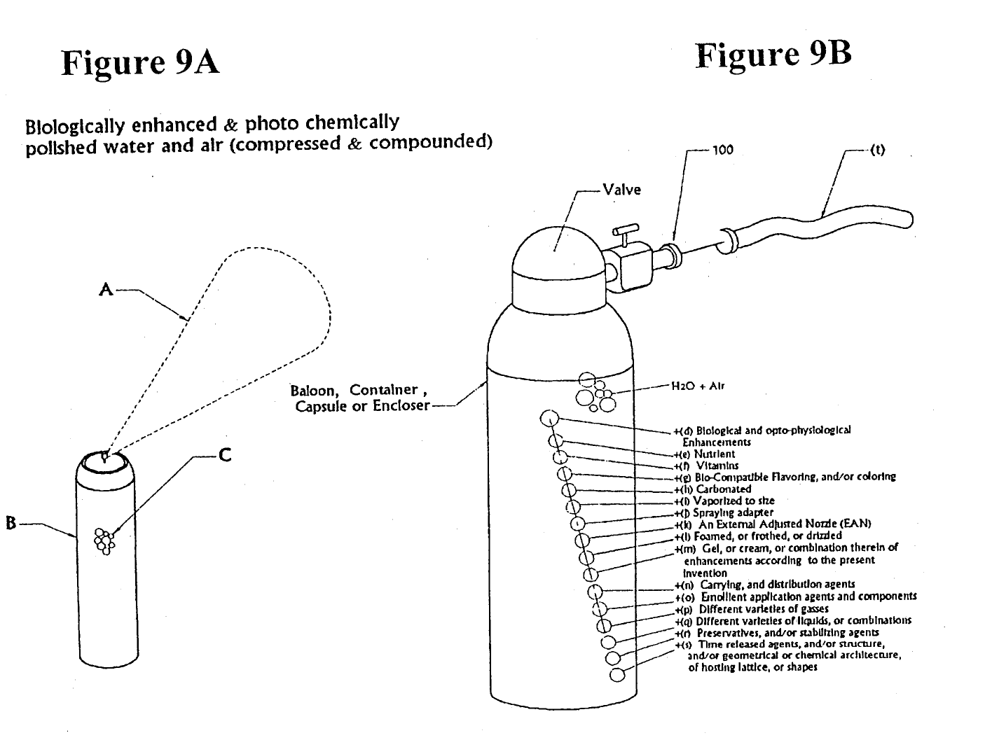

FIG. 9, A device storing the enhancements according to the methodology of the present invention is illustrated; A device for storing and applying the enhancements according to the present Invention is disclosed comprising a spray (ABC), (c) represent active traceable compound or substances according to the methodology of the present invention (see FIGS. 1-8), (A) represent the sprays content for (drinking and rubbing applications), (B) represent the holding bottle (compressed), or capsule, or containers holding said enhancements therein. An input to an external system, such as an externally connected mass production juice dispensers is incorporated for clarity, indicating that such enhancements according to the methodology of the present invention could easily interface to a wide varieties of water or air based, liquids, and/or gasses manufacturing processes, especially beneficial for strengthening the Human Immune System (HIS).

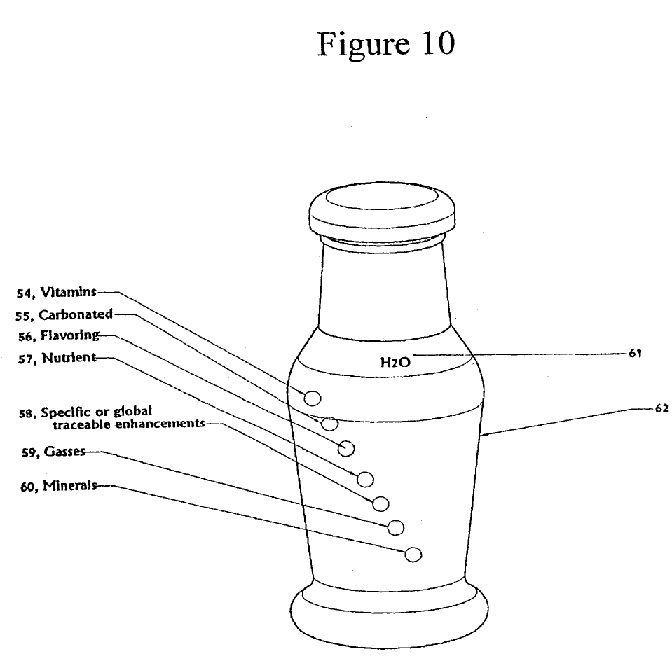

FIG. 10, illustrate a bottle holding, or storing enhancements according to the methodology of the present invention; A device holding biologically enhanced, and/or opto-physiologically improved, and/or photochemically polished drinking water is herewith presented comprising: (54) represent added vitamins, (55) illustrate added carbonated, (56) represent flavoring, (57) represent nutrients, (58) illustrate globally prepared, or specifically produced active ingredients (i.e. enhancements), (59) represent gasses variety, or individually gases added, (60) illustrates minerals added to the enhanced compound according to the methodology of the present invention, (61) represent H20, water as the base for the drinking enhanced compound of the present invention, (62) represent the holding, or storing bottle, or container, or capsule, pressurized, or stabilized, or simply storing said enhancements for later use, a valve is illustrated for clarity and to illustrate the flexibility of connecting, and dispersing, spraying, or releasing a predetermined amount of said enhancements according to the method of the present invention.

FIG. 11, illustrates, a fish held, and been breaded, immersed in the water, and said water are enhanced by the method of the present invention, said enhancement's active ingredients are showed in (64), (63) represent the fish's own immune system, (65) represent fish become the storing medium for the said enhancements (by being, and/or consuming said enhanced water), (67) represent an outline of the fish body itself which consumes, and live in said enhanced photochemically polished drinking water, or water for the fish to live in, (66) represent the global water environment of the fish, it is illustrated as already being mixed with enhancements according to the method of the present invention especially beneficial for the strengthening, improving, and enhancing human immune system (HIS), MHC, type 1, 2, 3), through eating said fish, once the fish has consumed sufficient amount of said enhancements, or for improving the fish's, own immune system, by feeding it said enhancements prepared especially from reverenced, or from noxious species which threatens the fish (or other animals, or humans, in brief both: strengthening the Human immune system (HIS) through eating, and/or consuming the fish, and/or strengthening the fish immune system through adding, or subtracting, and/or mixing said enhancements according to the methodology of the present invention.

Receiving in the context of the present invention means that the novel methodology of the present invention is able to accommodate, process (FIG. 1, 1-8/a-z) and compound liquids, gasses, and solids (FIG. 1, (1), 1-8/A-Z) at flow rates ranging from 1 litre per hour to about tens of Millions of gallons per day. More specifically the present invention preserve the natural characteristics of the source, aquifer, or feeding pumped input while turning noxious elements inherent in the bacteria, viruses and noxious microorganisms- into a beneficial, innocuous forms which tune, strengthened and enhance the reaction of the immune system of Humans, animals and plant.

Transferring through or by passing means in the context of the present invention that in accordance with specific water quality parameters, and the regulations, standards and requirements of specific application in the field, the liquids, gasses and solids may be passed through a prefiltration unit in order to barge the passage through of a certain particulate material sizes (0.1 Mic-1000 Mic).

Exposing in the context of the present invention means that the. liquids, or gasses or solids are exposed over a predetermined period of time for electromagnetic radiation or vibrations or transient cavitation or steady cavitation (>26 kHz T.C/s.c) or any light and sound combination (i.e. sonications) thereof.

Enhancing and strengthening in the context of the present invention that the noxious species present within said liquids and gasses or solids are becoming strong and/or replicating or biomass expanding due to the illumination or distribution of energies (i.e. such as light and sound/sonication), facilitating an accelerated proteins production and highlighting proteins yield or definitions.

Inactivating in the context of the present invention means that any bacteria present in the water, prior to selection, and subsequent processing (by the processes according to the present invention) is to remain alive for at least one stage, phase, or sphere, or for the entire duration of the process before inactivation occurs (FIG. 1, 1-8/a-z), and wherein inactivation is to occur, at least one generation, or 1 replication sequences of DNA & RNA have occurred previously in contradistinction to all disinfection and or conventional (prior) inactivation methodologies, such bio-mass expansion prior to inactivation is beneficial for processes according to the present invention augmenting the amounts from which biological traces or proteins, or amino-acids, or membranes, or photodamage products have being produced or secreted, extracted, separated, sorted and profuse phase converted without damage to the geometrical shapes of said biological traces using the synchronized and resolved electro-triatmic optical and physiological modulation capabilities of the method of the present invention.

More specifically, after inactivation occurs a bacteria that cannot replicate cannot infect that's why the present invention ensure that any derivatives or mix or compounding volume of the biocompound according to the present invention is safe at the output, or last stage in the process, and can be consumed for both domestically or commercially or for agriculture and irrigation applications wherein bio-organic produce is furnished without any threat of contamination by bacterium's which could replicate and therefor cause harm (i.e. no replication, no infection). Upon consumption of such biologically enhanced and polished water—the brain of the consommé or consumers retrieve data from the water or air being consumed, said water is being completely safe and have being made super safe by the processes according to the present invention, the immune system act and keep high level of alertness and awareness and so maintain its portfolio of potential reactions maintained and accessible at all times

Dissociating according to the context of the present invention means the separation, and dissociation by light and sound or mechanical means or combined synchronized or asymmetric interoperability (for the purpose if dissociation) combination of a predetermined selection of noxious species components (i.e. proteins, photodamage products, chemical triggers, cell membranes for example) in order to ensure long shelf life of the various proteins and also to determined the required concentration of the biocompound according to the present invention.

Transferring according to the novel methodology of the present invention means that any particulate materials forming during any of the processes according to the novel methodology of the present invention may be selectively removed by positioning a physical barrier above and/or below a certain threshold. More specifically such post filtration is especially beneficial for the production of liquids, or gasses having relatively high transparencies. More specifically such beneficial post-filtration unit may be required in accordance to standardization and

A triad Spheroid Electro-Triatmic drinking water Compound means in the context of the present invention, A spherical processor (FIG. 1, 1-8/a-z of the present invention) able to process simultaneously on more then one sphere able to process on three spheres (3) simultaneously or sequentially or serially or cyclically, or in parallel, or recurrently or non-recurrently water selected from a natural source or aquifer, or water which already been partially processed or wholly processed and wherein Triad represent “three” or (Tri-at-ma or trident, or trio) meaning three optional states, or three optionally beneficial phases, spheroid represent that any particular processes according to the present inventions may apear on more then one sphere, a sphere represent the three different states i.e. Liquids, Gasses, Solids and each of such sphere is therefor given its own processing chart or order or flow rate or production efficacy or combination. More specifically in the context of the present invention Triad spheroid Electro Triatmic means that the operation of the system is key for the production of the drink compound and its production sequences and flow charts operate simultaneously on three spheres, and wherein optical or physiological or biological manipulation in any given sphere is subject to maintaining source characteristics while expanding bio-mass and protein secretion through multi processing

legislation in any specific domestic, commercial and or agricultural applications wherein the novel methodology of the present invention is being utilized.

Acquired immunodeficiency Syndrom means In the context of the present invention A life threatening disease caused by virus and characterized by breakdown of the body's immune defenses.

Active immunity in the present invention means that the immunity produced by the body in response to stimulation by a disease causing organism or vacsine.

Agamaglobulinemia in the context of the present invention means An almost total lack of immunoglobulines, and/or antibodies.

Allergen in the context of the present invention means any substance that causes an Alergy, and/or Alergic reaction by the misfunctioning of the MHC type 1, 2, 3, and/or any combination thereof.

Alergy in the context of the present invention means An inappropriate and harmful responese of the immuna system to normally harmless substances (i.e see Alergic reaction as an example)

Anaphylactic Shoch means in the context of the present invention a life threatening Alergic reaction characterized by sweeling of the body tissues including the throat, dificulties in breathing, andd a suden fall in blood pressure.

Anergy means in the context of the present invention A state of unresponsivnes, induced when the T cell antigen receptor is stimulated, that effectivelly freezes T cell responses, pending a 2^{ND} Signal from the Antigen presenting cell illustrate the

A++ in the context of the present invention means; The programmability of acquired immune system by the intake of enhanced water, or air. It is directly related to the dynamics, kinetics of traceable recognition by the MHC type I, II, III, the distribution of immune components, and organ activities as well as to the level of cleanliness, i.e. the removal of toxic compounds (such as Selenium, Bromates, Boron and others) by altering their electronic, chemical, molecular composition, and structure, thus enabling to remove them, thus ensuring bio-compatibility for the traceable compounds according to the methodology of the present invention.

A++ also indicate the addition of a plurality of wavelength having the ability to agglomerate particles to larger sizes thus enabling to filter them, pre/post processing by the Electro-Triatmic Modulator (see ETM)

Antibody means in the context of the present invention a soluble protein mollecule produced and secreted by B cell in the response to an antigen, which is capable of binding to the specific antigen.

Antobody-dependent Cell—midiated Citotoxicity (ADCC) means in the context of the present invention An immune response in which antibody, by coating target cells makes them vandurable to attack by immune cell (see coating and marking cells)

Antigen means in the context of the present invention any substance that, when in troduced into the body is recognized by the immune system.

Antigen-presenting cells means in the context of the ptresent invention a B cells, cells of the monocyt lineage (including macrophages as s dantritic cells), and various other body cells that present antigen in a form that T cells can regognize.

Anti nuclear antibody (ANA) in the context of the present invention means An autoantibody directed against a substance in the cell nucleus.

Antiserum means in the context of the present invention a Serum that contains antibodies.

Antitoxins means oin the context of the prtesent invention Antobodies that in terlockk with and inactivate toxines produced by certain bacteria.

Appendix means in the context of the present ibnvention a lymphoide organ in the intenstine.

Attenuated means in the context of the present invention a weakend; no longer infecious (innocies form)

Autoantibody in the context of the present invention means an antibody that reacts against a persons own tissue.

Auoto immune desease in the context of the present invention means a desease that result when the immune system mistakenly attacked the bodies own tissues. For examplke the Rheumatoid arthritis and systemic lupus erythematosus aree auto immune deseases.

Bacterium in the context of the present invention means a microscopic noxious microorganizm composed of a singl cell. Mant but not all bacteria cause desease.

Bassophile means in the context of the present invention A white blood cell that contributes to inflamatory reactions. A long with mast cells, Basophiles are responsible for the symptomes of Alergy (and/or Alergic reactions).

B cells means in the context of the present invention a small white blood cells crucial to the immune defenses. Also known as B lymphocites, they are derived from bon Marrow And develope into plasma cells that are the source of antobodies.

Biological response modifiers means in the context of the present invention a substances, ither natural or synthesized, that boost, direct, or restor normal immune defenses. BRMs include interferons, interleukins, thymus hormones and monoclonal antibodies.

Biotechnology in the context of the present invention means the use of living organizms or their products to make or moodify a substance. Biotechnology includes recombinent DNA technique (such as genetic ingineering), and hybridoma technoilogy.

Bon-marrow in the context of the present invention means soft tissue located in the cavities of the bones. The bone marrow is the source of all blood cells.

Ceellular immunity in the context of the present invention means immune protection provided by the direct action of the immune cells (as distinct from soluble mollecules such as antibodies).

Chromosomes means in the context of the present invention physical structures in the cellls neuclies that have the genes. Each Human cell has 23 pares of chromosomes.

Clone in the context of the present invention means (n) A groupe of genetically identical cells or organlzms decended from a single common ancester., (v) to reproduce multiple identical copies.

Complement means ion the context of the present invention a complex series of blood proteins whos action complement the work of antibodies. Complement destroys bacteria produces inflamation, and regulate the immune reactions.

Complement cascade means in the context of the present invention a precize sequence of events usuallt triggered by an antigen/antibody complex in which each component of the comlement systen is activated in turnes.

Constanr region means in the context of the present invention that part of an Antibody structure that is characteristic for each antibody class.

Co stimulation means in the context of the present invention the delivery of a 2^{nd} signall from an antigen presenting cell to a T cel. The 2^{nd} signal rescues the activated T cell from Anergy, allowing it to produce the lymphokinds necessary for the growth of additional T cells.

Cytikines means in the context of the ptresent onvention a powerful chemical substances secrated by Cells. Cytokines include lymphokines produced by lymphocites and monokines produced by monycites and macrophages.

Dandritic cells means in the context of the present oinvention white blood cells found in the splin and other lymphoid organs. Dandritic cells typically use thread like tentacles to enmesh antigen, which they present to T cells.

DNA (deoxyribonucleic acid) means in the context of the present invention nucleic acid that is found in the cell nucleus and that is the carrier or co. represents genetic information. (i.e see RNA, Rebonucleic Acid or any combination thereof)

Enzyme means in the context of the present invention a protein, produced by living cells, that promotes the chemical processes of life without itself being altered.

Eosinophil means in the context of the present invention a white blood cell that contains granules filled with chemicals damaging to parasites, and enzymes that damp down inflammatory reactions.

Epitop means in the context of the present invention a unique shape or marker carried on antigen's surface, which triggers a corresponding antibody response.

Fungus means in the context of the present invention a member of a class of relatively primitive vegetable organism. Fungi include mushrooms, yeast, rusts, molds and smuts.

Gene means in the context of the present invention a unit of genetic material (DNA) that carries the directions a cell uses to perform a specific function, such as making a given protein.

Graft-versus-host disease (GVHD) means in the context of the present invention a life threatening reaction in which transplanted immunocompetent cells attack the tissues of the recepiant (such as in transplant medical proceadures).

Granulocytes means in the context of the present invention A white blood cell file with granules comtaining potent chemicals that allow the cell to digest micro organizms (noxious types and non noxious types) or to produce inflamatory reactions. Neutrophiles eosinophiles and basophiles are examples of granulocytes.

Helper T cells means in the context of the present invention A subset of T cells that tipically carry the T4 marker and are essential for turning on the antibody production, activating cytotoxic T cells, and innitiating many other immune responses.

Hematopoiesis means in the context of the present invention the formation and development of blood cells, usually takes place in the bone marrow.

Histo Compatibility Testing means in the context of the present invention A method of matching the self antigens (HLA) on the tissues of a transplant doner with those of the recipiant. The closer the natch, the better the chance that the transplant proceadure will be succesful “and will take”

HIV means in the context of the present invention (Human Immunodificiancy virus) it is the virus that causes AIDS.

Human lococyte antigens (HLA) means in the context of the present invention a protein in markers of celf use in histocompatibility testing. Soma HLA types also corolate with certain autoimmune deseases.

Humoral immunity means in the context of the present invention an immune protection provided by the soluble factors such as antobodies, which circulate in the body fluids or Humors, primarily Serum and lymph.

Hybridoma means in the context of the present invention A Hybrid cell created by fusing B lymphocytes with as long lived neoplastic plasma cell or T lymphocyte with a lymphoma cell. B cell Hybridoma secrates a singl specific antibody.

Hypogammmaglobuilinemia means in the context of the present invention is an abnormally low level of immunoglobuline.

Ideotypes means in the context of the present invention is a unique and characteristic part of an antibody variable region, which can dam cells serve as antigenes.

Immun Complex (IC) means in the context of the present invention a Claster of interleukines, antigenes and antibodies.

Immun response means in the context of the present invention the reactions of the immune system to forign substances.

Immunoassay means in the context of the present invention A test using antobodies to identify and quantify substances. Often the antibody is link to a marker such as a fluorecent mollecule, a radio active mollecule or an anzyme type, or any combination thereeof.

Immunocompetent means in the context of the present invention the capability of developing an immune response.

Immunoglobulines means in the context of the present invention a family of large protein mollecules also known as antibodies.

Immunosupression means in the context of the present invention A reduction of the immunresponses for instance by giving drugs to prevent transplant rejection/s.

Immunotoxines means in the context of the present invention A monoclonal antibody linked to a natural toxines, a toxic drugs or radio-active substance or combination.

Inflamatory respons means in the context of the present invention A redness, warmth, swelling, pain, and/or loss of function produced in response to infection as the result of increased flood flow and an oinfluxe of immune cells and secrations. I8nterleukines means in the context of the present invention A major group of lymphokines and monokines.

Kupffer cells means in the context of the present invention are a specialized macrophages in the liver.

LAK cells means in the context of the present invention A lymphocytes transformed in the laboratory into lymphokines activeted Killer cells which attack tumor cells.

Langerhans cells means in the context of the present invention A drandritic cells in the skine that pick up antigen and transport it to lymph nodes.

Leukocytes means in the context of the present invention an all white blood cells. Lymph means in the context of the present invention a transparent, slightly yellow fluide that carry lymphocytes, bathes the body tissues, and drains into the lymphatic vessels.

Lymphatic vessels means in the context of the present invention a bodywide network of channels, similar to the blood vessels, which transport lymph to the immune organs and into the bloodstream.

Lymph nodes means in the context of the present invention small bean-shaped organs of the immune system, distributed widely throughout the body and linked by limphatic vessels. Lymph nodes are garrisons of B, T, and other immune cells.

Lymphocytes means in the context of the present invention small white blood cells produced in the lymphoid organs and paramount in the immune defenses.

Lymphoid organs means in the context of the present invention the organs of the immune system, where lymphocytes develop and congregate. They include the bone marrow, thymus, lymph nodes, spleen, and various other clusters of lymphoid tissue. The blood vessel and lymphatic vessels can also be considered lymphoid organs.

Lymphokines means in the context of the present invention powerful chemical substances secreted by lymphocites. These soluble molecules help direct and regulate the immune responses.

Macrophages means in the context of the present invention a large and versatile immune cell that acts as a microbe-devouring phagocyte, an antigen-presenting cell, and an important source of immune secretions.

Major histocompatibility complex (MHS) means in the context of the present invention a group of genes that controls several aspects of the immune response. MHC genes code for self markers on all body cells.

Mast cells means in the context of the present invention a granule-containing cell found in tissue. The contents of mast cells, along with those of basophils, are responsible for the symptoms of allergy.

Mechanical Vibrations in the contex of the present invention relates to any type and property of mechanical vibrations in the range between 1 Hertz and 1 Tera Hertz.

Microbs means in the context of the present invention minute living organisms, including bacteria, viruses, fungi and protozoa.

Microorganisms means in the context of the present invention microscopic plants or animals.

Molecule means in the context of the present invention the smallest amount of a specific chemical substance that can exist alone. (the break a molecule down into its constituent atoms is to change its character. A molecule of water, for instance, reverts to oxygen and hydrogen).

Monoclonal antibodies means in the context of the present invention antibodies produced by a single cell or its identical progeny, specific for a given antigen. As a tool for binding to specific protein molecules, monoclonal antibodies are invaluable in research, medecine and infdustry.

Monocyte means in the context of the present invention a large phagocytic white blood cell which, when it enters tissue, develops into a macrophage.

Monokines means in the context of the present invention powerful chemical substances secreted by monocytes and macrophages. These soluble molecules help direct and regulate the immune responses.

Natural killer (NK) cells means in the context of the present invention large granule-filled lymphocites that take on tumor cells and infected body cells. They are known as natural killers because they attack without first having to recognize specific antigens.

Neutrophil means in the context of the present invention a white blood cell that is an abundant and important phagocyte.

Nucleic acids means in the context of the present invention large, naturally occuring molecules composed of chemical building blocks known as nucleotides. There are two kinds of nucleic acids, DNA and RNA.

OKT3 means in the context of the present invention a monoclonal antibody that targets mature T cells.

Opportunistic infection means in the context of the present invention an infection in an immunosuppressed person caused by an organism that does not usually trouble people with healthy immune systems.

Opsonize means in the context of the present invention to coat an organizm with antibodies or complement protein so as to make it palatable to phagocytes.

Organism means in the context of the present invention an individual living thing.

Paraasite means in the context of the present invention a plant or animal that lives, grows, and feeds on or within another living organizm.

Passive immiunity means in the context of the present invention immunity resulting from the transfare of antibodies or antiserum produced by another individual.

Peyer's patches means in the context of the present invention a collection of lymphoides tissues in the intenstinal tract.

Phagosites means in the context of the present invention a large white blood cells that contribute to the immune defenses by ingesting microbes or other cells and/or forign particles.

Plasma cells means in the context of the present invention a large antibody producing cell that develope from B cell.

Plaatelets means in the context of the present invention A granule containing a cellular fragments critycal for blood clotting and sealing of wounds. Plattelets also contributes to the immune respons.

Plymorphes means in the context of the present invention short for Poly Morpho Neuclear locosytes or granulocytes.

Proteins means in the context of the present invention an organic compound made up of aminoacids. Proteins are one of the major constituants of plants and animals and human cells.

Protozoa means in the context of the present invention a group of one celled animals, a few of which can cause Human desease (including maleria and sleepoing sikness types)

Rheumatoid factor means in the context of the present invention an auto antibody found in the serum of the most persones with rheumatoid artheritis.

RNA (Rebonucleic Acid) means in the context of the present invention a neucleic acid that is found in the cytoplasm and also in the neucleious of some cells. One function of RNA is to direct the synthesis of proteins.

Scavanger cells means in the context of the present invention any of the diverse group of cells that have the capability to engolfe and destroy forign materials, dead tissues, or other cells.

SCID mouse means in the context of the present invention a laboratory animal that, lacking anzyme necessary to fassion an immune system of its own, can be turned into a model of the human immune system when injected with human cells or tissues.

Serum means in the context of the present invention is a clear liquids that seperates from the blood when it is allowed to clott this fluids retains any antibodies that were present in the whole blood.

Severe combined immunodefficency desease (SCID) means in the context of the present invention A life thtreatening condition in which infants are born laccking all major immune defenses.

Splin means in the context of the present invention a lymphoid organ in the abdominal cavity that is important centre for immune system activities.

Stem cells means in the context of the present invention Cells from which all blood cells are derived. The bone marrow is rich in stem cells.

Suunit vaccine means in the context of the present invention a vaccine that uses marely one component of an infacious agent rather then the whole to stimulate an immune respons.

Supeantigens means in the context of the present invention a class of antigens, including certain bacterial toxines, that unleach a massive and damaging immune response.

Supressor T cells means in the context of the present invention a subset of T cells that turn off antibody production and other iimune response.

T cells means in the context of the present invention a small white blood cells that orchestrate and/or directly participate in the immune defenses. Also known as Tlymphocytes, they are proccesed in th thymus and secret lymphokines.

Thymus means in the context of the present invention a primary lymphoides organ, high in the chest where T lymphocite prolifirate and mature.

TIL means in the context of the present invention A TUMOR Infiltrating Lymphocytes. This immune cells are extracted from a tumor tissue, treated in laboratory and re-injected into the cancer patient.

Tissue typing means in the context of the present invention (see MHC, Major Histocompatibility Testing HCT).

Tolerance means in the context of the present invention a state of nonresponsivnes to a particular antigen or group of antigens.

Tonsils and adenoids means in the context of the present invention are a prominent oval massive of lymphoid tissues on either side of the throat.

Toxins means in the context of the present invention agents produced by plants and bacteria, normally very damaging to mammalian cells, that can be delivered directly to target cells by lionkiun gthenm to monoclonal antibodies or lymphokines.

Vaccine means in the context of the present invention a substance that contains antigenic components from an infectious organism by stimulating an immune response (but not disease) it protect against subsequent infection by that organism

Variable region in the context of the present invention means that part of an antibodies structure that differs from one antibody to another.

Virus in the context of the present invention means a sub-microscopic microbe that causes infectious disease. A virus can reproduce only in living cells.

Water Jet in the context of the present invention means a real time flowing liquid wave guid having any shape wherein light oscilates, locked therein and reflected by Total Internal Reflection (TIR).

Libido—pranic means in the context of the present invention a state of increased vitality (i.e. the word prana, pranic means vitality, or states of ample vitality), or such states in which the body is tuned, and the immune system is at rest, empowered by biological traces of specific origin, and nature. Libido is the sensual drive induced as a result of the body being tuned, and/or referenced, and systems in the body aren't over loaded, and thus a state of improved feelings is induced, i.e. a libido-pranic state.

Resonativistic means in the context of the present invention, a state of resonance created when energy of high peak powers is being applied, projected into, coupled to, or are being generated into biomass, such resonance is subject to the expansion ration, or density of the holding media, or medium, the term represent a state in which light and sound, ultrasound and mechanical, and physiological process are occurring and causing resonance which when said resonance utilizes the individual resonance of elements, organs, or cells of the noxious species, and could identify, or recognize, separate, sort and inactivate, dissociate, or vibrate said biomass,

For the purposes of this invention the following terms is included for the purpose of introducing the context (photochemistry context) for the benefits of the present invention could be easily and accessible explained, such terms and terminology in photochemistry mean:

Physical Constants of interest in Ultraviolet and Photochemistry means in the context of the present invention:

| Constant | Symbol | Value | Units |
|---|---|---|---|
| Speed of light | c | 2.99792458 × 10\hat{ }8 | M s \hat{ } (−1) |
| Charge on | e | 1.60217733 × | C |
| electron | | 10\hat{ }(−19) | |
| Planck constant | h | 6.6260755 × | J s |
| | | 10\hat{ }(−34) | |
| Boltzmann | k | k 1.380658 × | J K \hat{ } (−1) |
| constant | | 10\hat{ }(−23) | |
| Avogadro number | NA | 6.0221367 × | mol \hat{ } (−1) |
| | | 10\hat{ }23 | |
| Gas constant | R | 8.31451 | J mol \hat{ } (−1) K \hat{ } |
| | | | (−1) |

Further Characteristics of light and context explanatory notes are herewith included:

Planck Law of Radiation means in the context of the present invention:

Light which has both particle and wave properties. It is transmitted in discrete packets of energy (photons) and yet has a frequency and wavelength. The connection between these two properties is embodied in the Planck flaw of Radiation

Photochemical wave changes means in the context of the present invention:

The usual wavelength range in Photochemistry is 100-1000 nm (100.000-10,000 cm). Light photons with wavelengths longer than 1000 nm have a photon energy too

small to cause chemical change when absorbed, and photons with wavelengths shorter than 100 nm have so much energy that ionization and molecular disruptions characteristic of radiation chemistry prevail The total photochemical wavelength range is divided up into bands with specific names as given in Table 2.

Spectral ranges or interest in Photochemistry means in the context of the present invention:

| | Wavelength | Wavenumber | Energy Range |
|---|---|---|---|
| Range Name: | Range I nm | Range I cm | (kJ einstein˜′˜) |
| Near Infrared | 7(X) − I(XX) | 14.286 − I() O(X) | 120-171 |
| Visible | 400-700 | 25,000 14.286 | 171-299 |
| ultraviolet | | | |
| UVA | 315 400 | 31.746-25,000 | 299-380 |
| UVB | 2M0 315 | 35.714-31,746 | 3S0-427 |
| Vacuum Ultraviolet | 100-200 | 100,000-50,000 | 59$-1196 |
| (VUV) | | | |

Little photochemistry occurs in the Near Infrared. except for some photo-synthetic bacteria. which arc capable of storing solar energy at wavelengths out to 980 nm. The Visible range is completely active for photosynthesis in green plants and algae. Also many dyes can undergo photochemical transformations themselves or sensitize reactions in other molecules. Most studies in photochemistry involve the Ultraviolet range. The division into three sub-ranges is connected with the human skin's sensitivity to ultraviolet light. The UVA range causes changes in the skin that lead to sun tanning. The UVB range can cause sun burning and is known to eventually induce skin cancer. The UVC range is extremely dangerous since it is absorbed by proteins, RNA and DNA and can lead to cell mutations and/or cell death. The UVC range is sometimes called the germicidal range, since it is very effective in inactivating bacteria and viruses. The Vacuum Ultraviolet range is absorbed by almost all substances (including water and air). Thus it can only be transmitted in a vacuum. The absorption of a VUV photon causes one or more bond

breaks. However, even though photons with wavelengths less than 561.6 nm are capable of splitting the H2O2 molecule, no photolysis, or proteolysis occurs in this wavelength region because H2O2 does not begin to absorb ultraviolet light until below 300 nm. This illustrates the first Law of Photochemistry, namely that no photochemical reaction can occur unless a photon of light is absorbed.

Coherent and incoherent light means in the context of the present invention:

Light sources used in photochemistry can either be coherent (all emitted photons are in phase with each other as they propagate) or incoherent (all emitted photons have random phases). All lasers emit coherent radiation and usually at one wavelength. The dispersion is very small so that a laser beam remains at or near its original diameter as it propagates, The light emitted by all other light sources is almost always incoherent. Most of these sources are either “hot element” sources (e.g., the incandescent light bulb) or “plasma” sources (e.g., a fluorescent light tube).

Point sources, means in the context of the present invention:

Light sources have finite dimensions (e.g., often a cylindrical shape). Emission from such a source is difficult to treat mathematically. It is convenient to mode] these sources as a collection of point sources, in which all light is emitted from the point equally in all directions. The optics treatment for point sources is especially simple.

In the context of the present invention the Terms and concepts associated with the emission of tight, are herewith included for clarity of explanation and to simplify the understanding of the method of the present invention, especially wherein photochemistry is involved, or photochemical polishing is active in the processing according to the present invention:

The light emitted from a source can be viewed in many different ways. In this Section, the various terms that may be used to describe this emission are defined and explained.

Radiant energy in the context of the present invention means:

Radiant energy (Q) is a total amount of radiant emission (J) from a source over a given period of time,

Radiant power in the context of the present invention means:

The radiant power (P) of a source is the rate of radiant energy or total radiant power (W) emitted in all directions by a light source. For example, the radiant power of the Sun is 3-842×10²⁶ w. in theory, P, should include all wavelengths emitted by the source; however,˜is usually restricted to the wavelength range of interest to photochemistry. For example, if a light source is being used for ultraviolet photochemistry, P would be specified for emission in the 200-400 nm ultraviolet range.

Radiant power efficiency in the context of the present invention means:

The radiant power efficiency (q) is defined as

Q=P/e

where e is the input electrical power (W) supply.

Radiant emittance or excitance in the context of the present invention means:

The r Radiant emittance or excitance of a source is the radiant power emitted from an infinitesimal area on the surface of the source.

Radiant Intensity in the context of the present invention means:

The radiant intensity (I) (W sr\hat{ }(−1)) is the total radiant power P emitted by a source in a given direction about an infinitesimal solid angle.

Radiance in the context of the present invention means:

Radiance (L) is defined as the radiant power d²P, emitted from an infinitesimal area dA of the source surface in a given direction about the solid angle di, divided both by the solid angle di) and the orthogonally projected area.

The emittance M from an infinitesimal surface element dA is obtained by integrating L in spherical polar coordinates over the hemisphere of all outward-bound directions above dA.

An isotropic light source is defined as one in which the radiance L is uniform over all outward directions. Terms and concepts associated with the receipt of light When light is emitted from a source, it radiates outward at the speed of light, when it impinges on an object, it may be reflected, transmitted or absorbed. There are several terms that relate to the receipt of light.

Fluence Rate in the context of the present invention means:

Fluence Rate (E) (W m\hat{ }(-2) is the radiant power of all wavelengths passing from all directions through an infinitesimally small sphere of cross-sectional area d, divided, by

CM

Irradiance in the context of the present invention means:

Irradiance (symbol E; units W m\hat{ }(−2) is defined as the total radiant power of wavelengths incident on an infinitesimal element of surface of area as containing the point under consideration divided by as. The following are some important points regarding characteristics and differences between “irradiance” and “fluence rate”:

Examples: For a parallel and perpendicularly incident beam, not 5cattered or reflected, irradiance and fluence rate become identical. For any UV source within a three-dimensional volume, the integration of UV irradiance over the interior surface of the volume yields the UV power of the lamp., This is not true for UV fluence rate. The appropriate term for UV disinfection is “UV fluence rate” because a microorganism can receive UV power from any direction, especially when there is more than one UV lamp in the vicinity. In general usage, the irradiance or fluence rate may be expressed as MW cm\hat{ }(−2). The irradiance is often incorrectly termed “light intensity” see the proper definition of “radiant intensity” above.

Light dose or fluence in the context of the present invention means:

The light dose or fluence (symbol H. units J m˜2) is the total radiant energy of all wavelengths passing from all directions through an infinitesimally small sphere of cross-sectional area dA, divided by dA It is given by the average fluence rate times the exposure tune in seconds. The term UV dose is often used in UV disinfection literature. It represents the UV exposure of a given organism in the germicidal range.

Spectral units in the context of the present invention means:

All of the terms for tight emission or incidence refer to all relevant wavelengths. One can define spectral derivatives for each of these terms. For example. the light power emission of a LIV lamp is often expressed as the spectral power (W nm\hat{ }(−1), defined as the power output in a narrow wavelength band divided by the width of the band. The solar spectrum received at the Earth's surface is described in terms of the solar spectrum irradiance. Also the spectral distribution of a lamp emission is often given an a plot of spectral power versus wavelength.

Photon based units in the context of the present invention means:

Photo-chemistry involves the interaction of photons of light with molecules and in the context of the present invention means: the definitions units that are based on photons.

Photon irradiance, photon fluence rate and photon flow in the context of the present invention means:

Each of the spectral terms can be convened to a corresponding equivalent photon flow and fluence rate′ by dividing the term by the average photon energy in the narrow wavelength band.

Quantum yield in the context of the present invention means:

The quantum yield (unitless) Q is a measure of the photon efficiency of a photochemical reaction. e Is defined as the number of moles of product formed or reactant removed (P) per Einstein of photons absorbed

Line sources means in the context of the present invention:

When atoms are raised to an excited state, they emit only in very narrow lines with virtually no emission between the lines. The low-pressure mercury lamp is a very common lamp of this type. Table 3 gives the wavelengths and relative emittance for the emission lines of a low-pressure mercury vapor lamp.

Certain radiation units and associated light sources, (lasers) and lamps emit at longer wavelengths, This is the basis of the very popular fluorescent lamp. For example, The emission lines of a mercury lamp are only sharp when the pressure of the gas is low (<10 tore). If the pressure is increased, the lamp can carry much more power, but the emission lines broaden. For the same length of lamp (about 120 cm), a medium pressure lamp (pressure about 1000 torr) can carry up to 30,000W. These lamps are very common in commercial Systems utilizing ultraviolet light. FIG. 5 shows a comparison of the emission of low pressure and medium pressure lamps in the ultraviolet region.

Excimer lamps in the context of the present invention means:

Excimer lamps arc unique in that they emit in a narrow band of wavelengths. An excimer is an atomic dimmer that is stable only in the excited state and dissociates on decaying to the ground state. Table 4 gives the wavelengths of sonic of the common excimer lamps.

Examples: Emission wavelengths (or some common excimer lamps

| Excimer | Wavelength (nm) | Excimer | Wavelength (nm) |
|---|---|---|---|
| Xe2 | 172 | XeCl | 308 |
| KrCl | 222 | I2 | 342 |
| Cl2 | 259 | | |

Flash Lamps

Flash lamps are similar to continuous wave (CW), but could also operate in (PW), pulsed mode operation, and are lamps that consist of a cylindrical quartz tube with electrodes at each end and filled with a ga.½ (e.g. xenon). A power supply “fires” the lamps by discharging a large amount of electrical energy in a very short period of time (several us) by applying a very high voltage (10-30 kV}. The resulting plasma reaches temperatures of 10,000-13,000 K and the emission is essentially that of a blackbody (see FIG. 4). In commercial flash lamp Systems the lamp, a typical “flashed” about 30 times per s, but given a special electronic pulsing circuitry is added, repetition rates could reach Khz regime.

FEL in the context of the present invention, means Free Electron Laser and its derivatives, wherein space charged technologies (such as the Electrostatically Accelerated Free Electron Laser) include an electronic pulsing circuits, charging, or an accelerator (Such as R. F. Linac) is involved in the production of photons (around 100,000,000 photons per electrons, in contradistinction of a conventional crystal based laser having around 1 photon per electron, it is a laser which have less maintenance associated with its operation and its wall plug efficiency is reaching around the 40-51% respectively, and respectfully of the exact pumping geometry used.

The term EAFEL means in the context of the present invention Electrostaticely Accelerated Free Electron Laser and is an extremely efficient laser pumping geometry wherein recycling of the Accelerated electrons is being performed by utilizing deceleration techniques and its wall plug efficiency is estimated to reach in access of the 55% (amount of light being produced or converted from the electricity being consumed for its operation.

In the context of the present invention biologically enhanced or photo-chemically polished drinking water or breathing air means any liquid or gas which have been passed through or processed by the method of the present invention such as water and/or air), more specifically such processes involved in the polishing and enhancing may include; Optical inactivation, disinfection, inactivation of DNA and/or RNA replication sequences, Photocatalysis, electro catalysis, a hybrid of photo and electro-catalysis, optical dissociation, physiological dissociation, biomass expansion, filtration (pre/post), physical separation and sorting, reactivation, activation, sonication, acoustics, electroacoustics, electro-optical treatment (by photons of light), transgressing, or transversing said liquids and gasses through a photonic band gaped wave guides having an aerobic, non toxic passage for light and liquids or gasses or combination together, synchro and each separately or any combination thereof.

Peak power in the context of the present invention means, the energy generated when squeezing (i.e. such as when pulsing) electromagnetic energy in short duration of time, for example: a pulse of a given average energy and power—lasting, or having a pulse width of around 1 second (1s) will generate several watts in peak power, a pulse lasting or having width of microseconds(ms) will generate peak powers reaching the kilo-watts scale, while a pulse lasting nano (ns) seconds will generate peak powers reaching into the hundreds of million of watts which is especially beneficial for purposes such as optical dissociation, optical inactivation, optical polishing, and optical secretion and spectroscopy for control and diagnostics, so in short the shorter the pulse duration the higher its respective peak power.

Multi-photon-absorption-processes means in the context of the present invention a processes which when harnessed (such as in the method of the present invention) could be very beneficial for the photochemistry involved in the processing according to the present invention, for example when 10 mj of energy (250,000 photons) are projected into a liquid or gas, the time it takes this projection is very important for the process of the present invention, if these photons will be furnished over 1 second time domain, then it leaves sufficient time for the electrons in said liquid or gasses, to relax back to the relaxed state, but if we apply these photons in a time domain of 5 nano seconds, then we do not leave time for the electrons to relaxed and the processes is called Multiphoton absorption processes, this processes are non linear in nature and yield much higher quantum yield, or efficiencies, or speed of the reactivation, or a more efficient methodology for optical treatment, processing and polishing according to the present invention.

A hybrid of light sources means in the context of the present invention plurality of light sources and wherein their total spectral emitence, or total spectral distribution, or their total irradiance will cause multiphotonic absorption processes by means of super imposing their time domain (example: 1 light source is slow=1s pulse duration, and additional light sources are very fast=5 ns laser for example), their total irradiance is great and beneficial for the processes of the method of the present invention to occur efficiently, further more, such hybridization could includes, lamps and lasers, lasers and flash lamps, or any combination of CW, or PW type of light source working together, in synchronisity, and or sequentially or link or resolved by time domain manipulation for maximizing photonic interaction in matter.

Photo catalysis means in the context of the present invention: The use of energy of a photon of light to catalyze a chemical reactions. More specifically, such reaction may include the decomposition of water into hydrogen, and oxygen, and the complete oxidation of organic contaminants in aqueous environments. More specifically, the first step in photocatalysis is for the catalyst material to absorb photon of light in order to excite an electron from the velance band (VB), to the conduction band (CB), thus creating an electron-hole-pairs. Each Species must then migrate to the surface before recombination occurs. If this condition are met, the electron can be transferred to a surface adsorbed molecule, reducing it. The overall process is illustrated, it is important to note that for the processes to occur efficiently (preventing pre-matured recombination of the said electron-hole pairs), the rates of reduction, and the oxidation must be comperable. The position of the band edges is critical for each step of the process, in the context of the present invention a photocatalyst material which is stable in water is tio2, (or known as Titanium Oxide).

Electrocatalysis, similar to that explained in photo-catalysis, but instead of photons, an electrical charge is used, through the use of semiconductor material which has been specially selected (its band gap) for the charge applied, for the context of the present invention an electrocatalysis, stable in water is I.T.O, or known in its chemical name and signature Indium Tinoxide). Further more, it is especially beneficial to combine, and operate both electrocatalysis, and photo-catalysis simultaneously, or serially, or sequentially or in unison, or each separate catalytic is triggered separately or any combination thereof.

### DETAILED DESCRIPTION OF THE INVENTION

The novel methodology of the present invention discloses the provision of a novel production method for manufacturing especially prepared drinking water having Polly-Bio-Compound (PBC) (FIG. 1, 1-8/a-z) and said Biowater is capable of enhancing, rehearsing and tuning the immune systems of Humans and animals and be stored or held in plants for cyclic, seasonal or non recurrently held or distributed for quenching thirst by consumption. Further more, the present invention discloses a novel methodology for the immunifying of the immune system aspects through the introduction of a predetermined volume of photo-products derived in situ or added or subtracted to water, having an origin at least one cyclic, current, (1-8/a-z) or recurrent, (1-8/a-z) or, non-recurrent (1-9/a-z) replication sequences or phase change or combination. More specifically, the present invention provide for a beneficial mechanism for the creation (FIG. 1, 1-8/a-z) and economical manufacturing of a liquid drink compound based on water, causing improvements in maintenance command control and operation of the Human Immune System (HIS) once consumed. More specifically, such benefits induced by drinking or consuming the bio-compound according to the present invention causes enhancements to reception and recognition of compound (electro) chemical signaling, such recognition and subsequent required programming of the elemental according to the present invention is done by use of light and/or sound, and/or mechanical or physiological multiprocessing (FIG. 1, 1-8/a-z) and interaction and/or combinations according to the present invention. More specifically, such biologically enhanced drinking water which may be called Biowater (according to the present invention) in order to simplify the percussive lengthy textual presentation and portray the protective nature of the present invention using single word for so disclosing its plurality of benefits in short through the disclosing of a clearly defined novel methodology encapsulating control and manipulation of relevant time domain parameters associated with the production and delivery mixing and super excitation of said light causing controlled projection, emission, reflectance and wide range of flawless interactions by programming beams of light originated from population inversion or charge space technologies from about 1 electron per photon to about 1 Billion photon per single electron and wherein EAFEL, or FEL, or crystal based or gas discharge based or E-beam pumping and through predetermined absorption and coupling to bio-mass a predetermined fluence rate and intensities of origination, the present invention utilizes peak power of light to inactivate a predetermined volume or amount of noxious species or potential harmful invaders and dissociating said inactivated volume to proceed sequentially or physiological and wherein physically sorting said biologically enhanced drinking water for mixing, vapor, or vending or bottling or packaging or serving or storing, or temporarily or permanently distribute for later consumption, and wherein elements from noxious microorganism and other invaders presenting threats to the self, recognized immune system are being changed by light and being made innocuous and prompting and promoting body remedy. Especially beneficial for the production (1-8, FIG. 1 (a-z)) of a biologically enhanced drinking water free of toxic chemicals, beneficial for the immune system. Further more, such Biowater having a sufficient Polly-bio-compound traces therein to effect the immune system of humans animals or be stored in plants by means of dripping irrigation for accumulating and compounding volumes for further use or consumption. Especially beneficial to be accommodated in water or any liquids and/or gasses (and/or solids), or to be carried, delivered, distributed, stored, held or served and prepared for consumption through any combination thereof. The present invention provide a novel environmental protection for applications requiring updating of the population immune system, such task is beneficial and challenging and could be met according to the present invention by using simple water, and biomass expanding and treating, processing and converting said water to be biologically enhanced, containing traceable proteins, chemicals, organic, or non organic compounds, or photo-damage products, membranes, amino-acids or predetermined amounts of DNA, and RNA substances caused by time logged or stamped replication sequences. A further preferred embodiment according to the present invention wherein a particle reduction in incorporated or integrated, or is a part or complimenting any of the processing abilities of the separate modules, or conduits, or lasers, or ultrasound sources or guides, more specifically, an environmentally novel embodiment of the present invention for the production of biologically enhanced, encrypted, and uncorrupted polished and pure, potently traced and apportioned drinking water comprising:

Receiving a selected water input to a module having conduit, or chamber geometry and predetermined shape, or interface having at least one sphere of processing therein or thereafter and integral input global time reference per batch or volume a flow meter, and said module is having a predetermined volume and potential for accommodating biological mass expansion; Accommodating or pumping a predetermined feed, or biomass suspension or expansion or a source of liquids or gasses or any combinations into or through a predetermined conduit or chamber or module or biological settler having a predetermined volume, cross section and a geometry with at least one input and output, or inlet or outlet or combinations of terminals or integrated interfaces with option for onboard concentrators or cylindrical reactors; Transferring through, compressing or gating, or by passing or inter-modulating said liquids or gases or solids or any combination thereof through a semi opaque pre-filtration unit having a predetermined cut of or threshold or at least one membrane from about 0.1 Micron to about 1000 Micron, and said filtration unit is having a predetermined transmission capacity and cut-of threshold for the removal of large particulate materials; Exposing said liquids, or gases or solids or any combination thereof to at least one PW or CW light or sound or combination dose having a predetermined fluence rate, average energy, and predetermined intensity, as well as energy density, or combinations from at least one radiation unit having a high intensity light source and wherein at least one wavelength from the region from about 1 nm to about 3000 nm and energy from about 1 micro watt to about a few giga-wafts interferes and wherein at least one beam of light from the radiation unit is stirred for distribution by at least one optical fibers or photonic crystals and/or photonic band gaped aerobic waveguides or combinations or directly to said liquids, or gases or chamber or conduit or module; Dissociating mechanically, electronically, electro-optically, and/or acoustically or combination of said noxious species predetermined components, to form a predetermined volume of Biocompound containing a plurality of predetermined proteins, or photo-damage products; Transferring or phase converting—said bio-compound through a predetermined semi transparent or opaque, post filtration unit or membrane having a cut off from about 0.1 Micron to about 1000 Micron, or by-passing, compounding or mixing or converting said Polly-able biological compound into the gas state, liquid and/or solid state, and/or adjusting its relative temperature up to steam or down through condensation to about ice forming crystalline for morphemic choice of lattice hosting or tactically aurora aromatically tracing or calibrating continually or non recurrently said bio-able compound to its biological time referenced or reverenced source of origin; compounding or mixing or converting said Polly-able biological compound into the gas state, liquid and/or solid state, and/or adjusting its relative temperature up to steam or down through condensation to about ice forming crystalline for morphemic choice of lattice hosting or tactically aurora aromatically tracing or calibrating continually or non recurrently said bio-able compound to its biological time referenced or reverenced source of origin, thus each individually made volume of quantified bio able compound or any combination thereof in proportions is apportioned to represent an elemental of components, which when traced back in time for a period sufficiently long from about 2 to 18 DNA or RNA replication sequences back, for the purpose of furnishing synchronously said quenching bio able compound for human and animal consumption, by drinking and dripping or by irrigating and or temporarily or permanently, or prominently said compound is being held or distributed for storage and bottling, analysis or for feeding animals, or watering agricultural produce to be later consumed by man updating and enhancing consumer's immune system performance within a predetermined period of time.

1. A method for production of biologically enhanced drinking liquid or breathing gas, useful for immunizing by traceable recognition properties of the immune system of living being or plant, comprising; exposing a predetermined volume of liquid or gas or a combination thereof having a predetermined quantity of biomass components having traceable biologically noxious source or origin therein, to a light radiation emerging from at least one high intensity light radiation unit, or to mechanical vibrations emerging from at least one mechanical vibration oscillator unit, for a predetermined time interval wherein the energy amount of said light radiation, mechanical vibration, or a combination thereof, is being sufficient for the dissociation agglomeration or coagulation of at least a part of said traceable biologically noxious source or origin and insufficient for altering their geometrical integrity for future traceability by the immune system of human or of other creature or of a plant, upon consumption.

2. A method for production of biologically enhanced drinking liquid or breathing gas according to. Claim 1, further comprising the step of inserting a predetermined quantity of biomass components having traceable biologically noxious source or origin, into a predetermined volume of liquid or gas or a combination thereof, prior to exposing it to the light radiation or to the mechanical vibration.

3. A method for production of biologically enhanced drinking liquid or breathing gas, according to any one of the preceding claims, further comprising the step of dilution of a predetermined volume of the liquid or gas or a combination thereof with a predetermined volume of other biologically enhanced or non biologically enhanced liquid or gas or a combination thereof.

4. A method for production of biologically enhanced drinking liquid or breathing gas according to any one of the preceding claims, further comprising the step of detecting a time stamp positioning mark of the liquid or gas or a combination thereof, as a quality checkout or in order to determine their immunizing characteristics or for calibration.

5. A method for production of biologically enhanced drinking liquid or breathing gas, according to any one of the preceding claims, further comprising calibration of the light radiation properties by using a real time feedback of light originated within the treated liquid or gas as a result of the mechanical vibration activity, or calibration of the mechanical vibration properties by using a real time feedback of ultrasonic mechanical vibrations originated within the treated liquid or gas as a result of the light radiation activity, or any combination thereof.

6. A method for production of biologically enhanced drinking liquid or breathing gas, according to any one of the preceding claims, comprising; receiving predetermined volume of a global input of liquid or gases having a traceable biologically noxious source, or time stamp positioning mark; Accommodating or pumping simultaneously a predetermined feed of biologically and photo-chemically compatible, optically sensitive multi-component suspension or mix having a predetermined A++ action spectrum, refractive index, and conductive bandgap or barrier for charge hoping to occur in DNA or RNA composite components therein; transferring or projecting said liquids, and/or gasses, and/or solids or any jet combination thereof in a predetermined space, density or time so as to make it flow while absorbing light to glow; exposing said liquids, or gases or solids or any combination sequentially, cyclically, recurrently or non recurrently to at least one radiation unit having a high intensity source of light over a predetermined period of time; dissociating said multi-components multi-composite liquids, and/or gasses, and/or solids or combinations optically and transiently without critically altering their geometrical integrity over a predetermined period of time.

7. A method for production of biologically enhanced drinking liquid or breathing gas, according to any one of the preceding claims, comprising; receiving a global water, or liquids, or gasses, or any combination input having a time stamp, containing a known source of biological origin, (FIG. 1, 1-8/a-z, FIGS. 2-12, ref. 1-100) having a predetermined volume and potential for accommodating biomass expansion, and wherein said water, or liquids, or gasses, prior to reaching input phase selector has been cooled, heated, compressed or combination for conversion and adapting to be selected, or bypassed to be processed individually, and wherein said water, and/or liquids, and/or gasses is, or are having a predetermined transmission, or turbidity, or a predetermined level of suspended solids or TSS and/or biological or non biological substances or compounds of species specific proteins or calibration standards with predetermined photo-damage or dissociation resonativistic potential; accommodating or pumping a predetermined feed, or biomass suspension or expansion or a source of liquids or gasses or solids or any combinations into or through a predetermined conduit or chamber or module or biological settler having a predetermined volume, cross section and a geometry with at least one input and output, or inlet or outlet or combinations of terminals or integrated interfaces for interfacing, or interconnecting, or inter-operating with a control diagnostic router, and/or multi input and output expander with option for onboard concentrators or cylindrical and multi shapes reactors; transferring said liquids, and/or gasses, and/or solids or any combination thereof through (FIG. 1, 1-8/a-z, FIGS. 2-12, ref. 1-100) compressing or gating, or by passing or inter-modulating said liquids or gases or solids or any combination thereof through a semi opaque pre-filtration unit having a predetermined cut of or threshold or at least one membrane from about 0.1 Micron to about 1000 Micron, a transmission capacity and cut-of threshold for the removal of large particulate materials; exposing said liquids, or gases or solids or any combination thereof over a predetermined time accumulative parameters for bio-expansion (FIG. 1, 1-8/a-z, FIGS. 2-12, ref. 1-100), and opto-physiological, and opto-acousto enhancements by at least one pulsed wave or continuous wave, or transient type of light or sound energies or combination dose having a predetermined fluence rate, average energy, and predetermined intensity, as well as energy density, or combinations from at least one radiation unit interconnected, and/or inter-operated to at least one multi input, and/or output expander interface, and/or linked to control diagnostic router, and/or directly having a high intensity light source therein (i.e. for example: within a module, chamber, conduits, band gapped aerobic wave guide, or combination and wherein at least one wavelength from the region from about 40 nm to about 2400 nm and energy from about 1 micro watt to about a few giga-watts interferes, or being applied, or geometrically utilized and wherein at least one beam of light from the said radiation unit is stirred or directed for distribution by at least one optical fibers or photonic crystals and/or photonic band gaped aerobic waveguides or combinations or directly to said liquids, or gases or chamber or conduit or module or any combination thereof or said liquids or gasses, or light, or combination may flow throughout the said guides or directly be compressed or freely flow by gravitation or atmospheric pressure and/or pumping; enhancing and strengthening and expanding repair activity, proteins production, and/or definition yields, and/or by acousto-optic, or sonic, and/or photo-reactivation of the noxious microorganisms contained within said liquids and gasses or any combination over a predetermined period of time from about 1 Millisecond to about 24 hours; inactivating said liquids, or gases or solids or any combination thereof by delivering directly, and/or by transferring by photonic band gapped, or conventional high grade fused silica (HGFS), or quartz, or sapphire, or polymer, or organo-elastomers or combination of optical fibers, or non toxic aerobic liquid light guides, at least one pulsed wave, and/or continuous wave, and/or transient type of light or sound, and/or sonic energies or combination dose having a predetermined fluence rate, average energy, and predetermined intensity, as well as energy density, or combinations from at least one additional radiation unit interconnected or inter-operated, or interfaced to said multi input, output expander interface, and/or linked to said control. and diagnostic router (CDR), DNA and/or RNA replication sequences of specific noxious species by electro-photochemical, and/or electro-optical, and/or electro optocatalytic processes (FIG. 1, 1-8/a-z, FIGS. 2-12, ref. 1-100), and/or Non thermal plasmas, and/or Electron-beam, and/or Y-rays, and/or Supercritical water Oxidation, and/or Electrohydraulic Cavitation & Sonolysis, and/or Photocatalytic Redox Processes, and/or H2O2/UV, and/or H2O2/O3, and/or O3/UV, variant oxidation agent and/or any combinations thereof (FIG. 1, 1-8/a-z, FIGS. 2-12, ref. 1-100); dissociating said liquids, and/or gasses, and/or solids or combinations, optically in optical dissociation module (FIG. 1, 1-8/a-z, FIGS. 2-12, ref. 1-100) by delivering directly, and/or by transferring by photonic band gapped, or conventional high grade fused silica (HGFS), or quartz, or sapphire, or polymer, or organo-elastomers or combination of optical fibers, or non toxic aerobic liquid light guides, at least one pulsed wave, and/or continuous wave, and/or transient type of light or sound, and/or sonic energies or combination dose having a predetermined fluence rate, average energy, and predetermined intensity, as well as energy density, or combinations from at least one additional radiation unit interconnected or inter-operated, or interfaced to said multi input, output expander interface, and/or linked to said control and diagnostic router (CDR), and/or mechanically, electronically, electro-optically, and/or acoustically and/or sonically or combination (FIGS. 1-12, ref. 1-100) for physical separation of said noxious species predetermined components, to form a predetermined volume of Bio-compound containing a plurality of predetermined traceable proteins, or photo-damage products; transferring (FIG. 1, 1-8/a-z, FIGS. 2-12, ref. 1-100), or phase converting and routing said bio-compound enhancements through a predetermined semi transparent or opaque, post filtration unit or module, or membrane or combination having a cut off from about 0.1 Micron to about 1000 Micron, or by-passing, compounding or mixing or converting said Polly-biological compound into the gas state, liquid and/or solid state, and/or adjusting its relative temperature up to steam or gradually, or quickly down through condensation to about ice forming, or heating, cooling or compressing, or for combinations thus making a crystalline for morphemic choice of lattice hosting, or tactically aurora aromatically tracing or calibrating continually or non recurrently said bio-able compound to its biological time referenced or reverenced source of origin, or time stamp, thus each individually made volume of quantified bio able compound or any combination thereof may be processed prior to processing by modules, or spheres of the present invention, by variety of cross platform processes selected from electro-photochemical, and/or electro-optical, and/or electro opto-catalytic processes, and/or Non thermal plasmas, and/or Electron-beam, and/or Y-rays, and/or Supercritical water Oxidation, and/or Electrohydraulic Cavitation & Sonolysis, and/or Photocatalytic Redox Processes, and/or H2O2/UV, and/or H2O2O3, and/or O3/UV, and/or any combinations thereof in proportions, is apportioned to represent an elemental of components, which when traced back in time for a period sufficiently long from about 2 to 18 DNA or RNA replication sequences back, for the purpose of furnishing synchronously said quenching bio able compound for human and animal consumption, by drinking and dripping or by irrigating and or temporarily or permanently, or prominently said compound is being held or distributed for storage and bottling, analysis or for feeding animals, or watering agricultural produce to be later consumed by man updating and enhancing consumer's immune system performance within a predetermined period of time.

8. A method for production of biologically enhanced drinking liquid or breathing gas, according to any one of the preceding claims, comprising; receiving a selected water input to a module having conduit, or chamber geometry and predetermined shape, or interface having at least one sphere of processing therein and when each of said spheres is able to perform at least one process involving oxidation at high resolution of accuracy using at least one stage of processing according or selected from electro-photochemical, and/or electro-optical, and/or electro optocatalytic processes, and/or Non thermal plasmas, and/or Electron-beam, and/or Y-rays, and/or Supercritical water Oxidation, and/or Electrohydraulic Cavitation & Sonolysis, and/or Photocatalytic Redox Processes, and/or H2O2/UV, and/or H2O2/O3, and/or O3/UV, and/or UV, or any oxidizing agent, or any combinations thereof, and whereby during said processes, or thereafter, and integral to said processes or combinations, each of said spheres or modules provides for input global time reference, or time stamp per batch or volume, or quantities, and a flow meter is incorporated globally or specifically per module, and said module is having a predetermined volume and potential for accommodating biological mass expansion, and wherein said water is having a transmission, or turbidity, or a predetermined suspended solids or TSS and/or biological or non biological substances or compounds of species specific biological origin extending at least one replication sequences back in time and said compound contain oxygen, or proteins or Hydrogen, or vitamins, or minerals, or nutrients, or carriers having known predetermined particle size distribution PSD, and predetermined quantum yield of transfer efficiency or calibration standards for transferee and wherein additional variable volume is introduced from an external satellite module, or external processing module, having identical geometrical utilization and processing capabilities for proximity or remote interoperability for synchronized operation with said receiving module and wherein its content is locked or reference or reverence by diagnostics, resolving predetermined photo-damage or dissociation, or excitation thus effecting and causing resonativistic potential to manifest, or materialize leaving an impression or imprint of sorted origin of biological source or input for later recognition; Accommodating or pumping a predetermined feed, or biomass suspension or expansion or a source of liquids or gasses or any combinations into or through a predetermined conduit or chamber or module or biological settler having a predetermined volume, cross section and a geometry with at least one input and output, or inlet or outlet or combinations of terminals or integrated interfaces with option for onboard concentrators or cylindrical reactors; Transferring through, compressing or gating, or by passing or inter-modulating said liquids or gases or solids or any combination thereof through a semi opaque pre-filtration unit having a predetermined cut of or threshold or at least one membrane from about 0.1 Micron to about 1000 Micron, and said filtration unit is having a predetermined transmission capacity and cut-of threshold for the removal of large particulate materials; Exposing said liquids, or gases or solids or any combination thereof to at least one PW or CW light or sound or combination dose having a predetermined fluence rate, average energy, and predetermined intensity, as well as energy density, or combinations from at least one radiation unit having a high intensity light source and wherein at least one wavelength from the region from about 1 nm to about 3000 nm and energy from about 1 micro watt to about a few giga-watts interferes and wherein at least one beam of light from the radiation unit is stirred for distribution by at least one optical fibers or photonic crystals and/or photonic band gaped aerobic waveguides or combinations or directly to said liquids, or gases or chamber or conduit or module or any combination thereof or said liquids or gasses flow throughout the said guides or directly compressed or freely flow by gravitation or atmospheric pressure; Enhancing and strengthening and expanding repair activity, proteins production and/or definition yields, and/or by acousto, or sono-optic-photo-reactivation of the noxious microorganisms contained within said liquids and gasses or any combination over a predetermined period of time from about 1 Millisecond to about 24 hours; Inactivating DNA and/or RNA replication sequences of specific noxious species by electro-photochemical, and/or electro-optical, and/or electro opto-catalytic processes or any combinations thereof; Dissociating mechanically, electronically, electro-optically, and/or acoustically or combination of said noxious species predetermined components, to form a predetermined volume of Biocompound containing a plurality of predetermined proteins, or photo-damage products; Transferring or phase converting—said bio-compound through a predetermined semi transparent or opaque, post filtration unit or membrane having a cut off from about 0.1 Micron to about 1000 Micron, or by-passing, compounding or mixing or converting said Polly-able biological compound into the gas state, liquid and/or solid state, and/or adjusting its relative temperature up to steam or down through condensation to about ice forming crystalline for morphemic choice of lattice hosting or tactically aurora aromatically tracing or calibrating continually or non recurrently said bio-able compound to its biological time referenced or reverenced source of origin, thus each individually made volume of quantified bio able compound or any combination thereof in proportions is apportioned to represent an elemental of components, which when traced back in time for a period sufficiently long from about 2 to 18 DNA or RNA replication sequences back, for the purpose of furnishing synchronously said quenching bio able compound for human and animal consumption, by drinking and dripping or by irrigating and or temporarily or permanently, or prominently said compound is being held or distributed for storage and bottling, analysis or for feeding animals, or watering agricultural produce to be later consumed by man updating and enhancing consumer's immune system performance within a predetermined period of time.

9. A method for production of biologically enhanced drinking liquid or breathing gas, according to any one of the preceding claims, comprising; receiving a selected liquid or gasses input source having a predetermined volume and generative potential for photochemical polly-interactive, photonically induced to represent or trace a predetermined geometry or chemistry or compound having at least one DNA and or RNA replication sequence proliferated for bio-mass expansion; accommodating an additional volume of water congenitally identical in at least one parameter or having a predetermined flow rate characterized by mixing parameters (FIG. 1, 1-8/a-z) or quality parameters, or Current, or cyclic, or recurrent, or non recurrent, or replication sequences of DNA and/or RNA, or, waved continuum substrate of predetermined volume encapsulating remains of photo-damaged portfolio of substances and proteins augmenting by photochemical reaction synthesis including at least one process selected from by electro-photochemical, and/or electro-optical, and/or electro optocatalytic processes, and/or Non thermal plasmas, and/or Electron-beam, and/or Y-rays, and/or Supercritical water Oxidation, and/or Electrohydraulic Cavitation & Sonolysis, and/or Photocatalytic Redox Processes, and/or H2O2/UV, and/or H2O2O3, and/or O3/UV, and/or any combinations thereof introducing. photons which geometrically or Geo-genetically utilizes time domain for optically command controlling compounding volumes of proportionate proteins, membranes, and innocuous substances of. parts and combinations of components therein, and wherein electro-optically secreted physiologically or mechanically barred or sorted and routed, or converted by flawless continuum of photochemical reactions across at least two spheres in one or more order of interaction, or in at least one state, or phase or combinations states or stages of phases by activation, or reactivation of repair mechanism of noxious species definition of source in accordance with the characterization of original inlet or source wherein noxious species remains originate the frame work of which quality parameters are determined of said liquids, or gasses, or solids; volumes of bio-mass of species specific proteins or membrane combinations are being mixed or stored or packed, or vapor vended, or produced and distributed for agriculture for commercial re-consumed economically viable calibration standards with predetermined photo-damage or dissociation potential, or resonance, or polyphonically transient cavitationed for simultaneously or sequentially, serially or in parallel, producing said bio-compound and illuminating or irradiating it over a predetermined space over a predetermine period of time thus charging it for consumption; Accommodating or pumping a predetermined feed, or bio-mass suspension or expansion or a source of liquids or gasses or solids or any combinations into or through a predetermined conduit or chamber or module modeled for having a predetermined volume, cross section and at least one input and output or inlet or outlet or combinations of terminals or integrated interfaces; Transferring through or by passing or intermodulating or mixing said liquids or gases or solids or any combination thereof through a semi opaque pre-filtration unit having a predetermined cut of or threshold from about 0.1 Micron to about 1000 Micron for the removal of large particulate materials; Exposing said liquids, or gases or any combination thereof to at least one Pulsed wave (PW) or Continuos Wave (CW) light or sound or combination dose having a predetermined fluence rate, average energy, and predetermined intensity, as well as energy density, or combinations from at least one radiation unit having a high intensity light source and wherein at least one wavelength from the region from about 1 nm to about 3000 nm and energy from about 1 micro watt to about a few Gigawatts and wherein light from the radiation unit is distributed by at least one optical fibers or photonic crystals and/or photonic band gaped aerobic waveguides or combinations or directly to said liquids, or gases or solids or chamber or conduit or any combination thereof; Enhancing and strengthening and expanding repair activity, proteins production and/or definition yields, and/or photo-reactivation of the noxious microorganisms contained within said liquids and gasses or solids or any combination over a predetermined period of time from about 1 Millisecond to about 24 hours; Inactivating DNA and/or RNA replication sequences of specific noxious species by electro-photochemical, and/or electro-optical, and/or electro optocatalytic processes or any combinations; Dissociating and secreting mechanically, electronically, electrooptically, and/or acoustically or combination of said noxious species predetermined components, to form a predetermined volume of Biocompound containing a plurality of predetermined proteins, or photo-damage products or sufficient representation; Transferring or phase converting—said Biocompound through a predetermined semi transparent post filtration unit having a cut off from about 0.1 Micron to about 1000 Micron, or by-passing, compounding or mixing or converting said polybiological compound into the gas state, and/or liquid state, and/or solid state or any combination thereof in proportions for the purpose of furnishing said Biocompound for consumption, and or temporarily or permanently held for storage and or for feeding agricultural produce to maintain said compound characteristic causing immune enhancements to be later consumed by man to that effect.

10. A method for production of biologically enhanced drinking liquid or breathing gas, according to any one of the preceding claims, comprising; receiving or accommodating a selected gasses or liquid mix, or a predetermined source of water having known turbidity, Total Suspended Solids (TSS) or Biological compounds, or non biological compounds, or particle size distribution (PSD), OR transmission, or UVT, or absorption, or combination containing a predetermined dissolved oxygen or flavored or diluted, or enhanced for consumption input—source, or aquifer r for mineral, fresh or spring water having a predetermined volume, as well; as generative potential for poly-phonically, or multi-phonically, or multi photonically accommodating an additional volume congenitally identical in at least one parameter or having a predetermined individual flow rate characterized by its own quality parameters and when mixed, diluted or combined yield (A/0) Current, or (A/1) cyclic, or (A/2) recurrent, or (A/3) non recurrent, or (A/4) replication sequences of (A/5) DNA and/or (A/6) RNA, or, (A/6) waved remains of photo-damaged portfolio of products and augmenting by (A/7) photochemical reaction synthesis (A/8) introducing photons which geometrically or geo-genetically utilizes (A/9) time domain for optically command controlling compounding volumes of proportionate proteins, membranes, and innocuous substances and combinations which are (A/10) electrooptically secreted, or mechanically barred or sorted and routed by flawless continuum of photochemical reactions across at least two spheres in at least two order interaction or more, or in at least one state, or phase or combinations states or stages of phases by activation, or reactivation of repair mechanism of noxious species definition of source and characterizing of original inlet and wherein noxious species remains by vibrations (FIG. 1, 1-8/a-z) the frame work of quality parameters of said liquids, or gasses; volumes of bio-mass of species specific proteins or membranes combinations are being mixed or stored or packed, or vapor vended, or produced (FIG. 1, 1-8/a-z of the present invention) and distributed for agriculture for commercial re-consumed economically viable calibration standards with predetermined photo-damage or dissociation potential, or resonance, or polyphonically transient cavitationed for simultaneously or sequentially, serially or in parallel, producing said bio-compound and illuminating or irradiating it over a predetermined space over a predetermine period of time thus charging it for consumption; Accommodating or pumping a predetermined feed, or bio-mass suspension or expansion or a source of liquids or gasses or solids or any combinations into, or through a predetermined conduit or chamber or module modeled for having a predetermined volume, cross section and at least one input and output or inlet or outlet or combinations of terminals or integrated interfaces; Transferring through or by passing or intermodulating said liquids or gases (or solids) or any combination thereof through a semi opaque pre-filtration unit having a predetermined cut of or threshold from about 0.1 Micron to about 1000 Micron for the removal of large particulate materials; Exposing said liquids, or gases or solids or any combination thereof to at least one Pulsed wave (PW) or Continuos Wave (CW) light or sound or combination dose having a predetermined fluence rate, average energy, and predetermined intensity, as well as energy density, or combinations from at least one radiation unit having a high intensity light source and wherein at least one wavelength from the region from about 1 nm to about 3000 nm and energy from about 1 micro watt to about a few Gigawatts and wherein light from the radiation unit is distributed by at least one optical fibers or photonic crystals and/or photonic band gaped aerobic waveguides or combinations or directly to said liquids, or gases or solids or chamber or conduit or any combination thereof; Enhancing and strengthening and expanding repair activity, proteins production and/or definition yields, and/or photo-reactivation of the noxious microorganisms contained within said liquids and gasses or solids or any combination over a predetermined period of time from about 1 Millisecond to about 24 hours; Inactivating DNA and/or RNA replication sequences of specific noxious species by electro-photochemical, and/or electro-optical, and/or electro optocatalytic processes or any combinations; Dissociating and secreting mechanically, electronically, electrooptically, and/or acoustically or combination of said noxious species predetermined components, to form a predetermined volume of Biocompound containing a plurality of predetermined proteins, or photo-damage products; Transferring or phase converting (FIG. 1, 1-8/a-z) said Bio-compound through a predetermined semi transparent post filtration unit having a cut off from about 0.1 Micron to about 1000 Micron, or by-passing, compounding or mixing or converting said polybiological compound into the gas state, and/or liquid state, or jelly, or amorphous state or any combination thereof in proportions for the purpose of furnishing said air based Biocompound for consumption, and or temporarily or permanently held for storage and analysis or for feeding agricultural produce to be later consumed and breathed by man, animal and plants.

11. A method for production of biologically enhanced drinking liquid or breathing gas, according to any one of the preceding claims wherein tracers, markings, signaling or essential proteins, and membranes, and photodamage products are stored, or held temporarily or permanently in water droplets, and wherein said droplets are released or vaporized through, and aerated, or vaporized into and from liquids, or beverages, or syrups or juice dispenser, or syrup dispensing units, or bottles of mineral or spring waters (FIG. 1, 1-8/a-z), or biologically enhanced drink and beverages production sites and manufacturing plants as well as natural and man made aquifers.

12. A method for production of biologically enhanced drinking liquid or breathing gas, according to any one of the preceding claims, wherein liquids or gasses or solids which have been processed (FIG. 1, 1-8/a-z) and or partially or sequentially or cyclically (1-8), (a-z) pass, or been selected, or filtered, or illuminated or irradiated from about 1 nm to about 3000 nm through each and/or any combination of the processing (distribution) chart, may be beneficial for consumption by humans, and or for distribution into agriculture, dripping irrigation and vegetation and fruit volume or matrix varieties for the purpose of being consumed through foods and drinks, medical, and commercial agricultural produce facilitated by using the method of the present invention (FIG. 1, 1-8/a-z).

13. A method for production of biologically enhanced drinking liquid or breathing gas, according to any one of the preceding claims, wherein a bottling plant for producing mineral or spring water is selected as a source and wherein said biologically enhanced water is mixed with a predetermined quantity of selected additives or flavors, or form base from which new beverages or syrups or nutrient compliment beneficial architecture or components or dilution or wherein said added or integrated profuse or infused, inserted, or subtracted aid in the carrying or distribution maximizing the time span from which the benefits of uniquely impersonated, biologically enhanced drinking water is being felt after consumption occurs.

14. A method for production of biologically enhanced drinking liquid or breathing gas, according to any one of the preceding claims, wherein modules, or conduits, or chambers, or external satellite processing module are interpreted or logged on time track or referenced, interconnected and/or inter-operated in any order, such as for serial, or parallel, or for array architecture and wherein the modular potential of devices making use of, or operating according to the method are processing a predetermined volume taking into account the required bio-mass-expansion and flow rates, and bypasses, or treatability is measured to coincide with quantities, and quantified proportions are then distributed for packing, vending, vaporizing, and bottling said biologically enhanced drinking water for consumption over a predetermined period of time from the time reference of source, origin, or time stamp, or any of said modules maximizing shelf life of biowater according to the method of the present invention.

15. A method for production of biologically enhanced drinking liquid or breathing gas, according to any one of the preceding claims, wherein the light radiation involved in the production processes is emerging from any light source or radiation unit having a high intensity source of light from about 1 microwave to about few giga-watts, and wherein its fluence rate, average energy, and spectral distribution is encapsulating at least one region of the electromagnetic spectrum from about 40 nm to about 4000 nm, and wherein its spatial characteristics including continues wave operation or pulsed wave operation and wherein operating in pulses is selected or programmed, time domain is thus controlled, triggered or referenced to biological time input reference and each pulse in a burst or from a continuum of repetition rates is lasting from about 1second to about 1picosecond, or from 7 microseconds to around 1 femtosecond, having per shot peak powers reaching from the region of about 1 nano joule, to about 22 joules, per pulse respectively and when resolved to biological input time reference and extrapolated will guide dosimeters or values for which curves effecting the concentration or dilution, expansion or recognition scope or velocities associated with opto-mechanically, electro-optically, photo-catalysis, or electro-catalysis as involved in the method according to he present invention, especially beneficial for the enhancement of the human immune system by consumption of a predetermined proportions, apportions in accordance with specie specific calibration standards therein.

16. A method for production of biologically enhanced drinking liquid or breathing gas, according to any one of the preceding claims, wherein vitamins or nutrients are being added, or subtracted, or enriched or complimenting said biological enhancements, and wherein said vitamins or nutrients are enhanced for the provision of carrying or distributing said biologically enhanced water for generally and particularly promoting health of the immune system chemical activity, production yields, or generally referenced state.

17. A method for production of biologically enhanced drinking liquid or breathing gas, according to any one of the preceding claims, wherein at least one module, or conduit, or chamber or array of a geometry used in the production process is coated with at least one semi-conductor having stabilized or destabilized for the provision of photo-catalysis, or electro-catalysis or any combinations thereof or sequentially or polyphonically operated in unison, or resolved and time referenced, or reverenced for maximizing safety of operation and inter-operability of devices using the method of the present invention.

18. A method for production of biologically enhanced drinking liquid or breathing gas, according to any one of the preceding claims, wherein at least one light source involved in a photochemistry process during the production is a flush lamp type of light source, and wherein at least one beam of light from said light source is in the region from about 40 nm to about 2400 nm, and wherein its respective time domain is from about 1picosecond to about 5 ns, and wherein each individual pulse generates energy in access of 250 milijoul per pulse, or more then 250 milliwatt per second, and wherein the spectral relevance for the processes of the present invention encapsulating the IR, visible, UVA, or UVB, UVC, OR VUV, or any combination thereof, or at least one process selected from electro-photochemical, and/or electro-optical, and/or electro optocatalytic processes, and/or Non thermal plasmas, and/or Electron-beam, and/or Y-rays, and/or Supercritical water Oxidation, and/or Electrohydraulic Cavitation & Sonolysis, and/or Photocatalytic Redox Processes, and/or H2O2/UV, and/or H2O2O3, and/or O3/UV, and/or any combinations thereof have been triggered, induced, activated, inactivated, turned off, or accented, reduced, or increased, or controlled synchronously for biomass expansion, or for inactivation, or for dissociation, or for sorting, or apportioning, portioning, diluting, and or mixing, reducing and increasing components volumes within compound or stabilizing, or sterilizing said biological, and optophysiological, or combinations of photochemical and electrooptical altering and enhancing drinking water, and air for breathing for the purpose of consumption.

19. A method for production of biologically enhanced drinking liquid or breathing gas, according to any one of the preceding claims, wherein at least one external module is being linked, or synchronized, interconnected, or inter-operated or referenced, or reverenced, or monitored, or incorporated into the production processes.

20. A method for production of biologically enhanced drinking liquid or breathing gas, according to any one of the preceding claims, wherein modules involved in the production process are arranged in parallel, or in serial configurations or a combination thereof.

21. A method for production of biologically enhanced drinking liquid or breathing gas, according to any one of the preceding claims, wherein a phase router is used in the production process and the output of the phase router is encapsulated within at least one bottle, or bag, or conduit or chamber for consumption in a predetermined prepackaged portions, or apportioned to be distributed to at least one vegetable selected from water containing vegetation, or fruits, or being distributed or delivered, or carried or spread using drip irrigation or agricultural diffusers for the purpose of harboring said biologically enhanced drinking water in plants prior to consumption of fruit thereof especially beneficial for the consumption of biowater according to the present invention as part of the daily diet of individual end users, or producers, thus strengthening their immune system by utilizing intake in drinking and in food separately or synchronously or in according with preference or, reverence used for calibration against species specific noxious qualities or geometry derived thereof or in accordance with variable availability of each components according to the method of the present invention, or in any combination thereof.

22. A method for production of biologically enhanced drinking liquid or breathing gas, according to any one of the preceding claims, wherein a settler is used for the expansion of bio-mass during the production process and is utilizing at least one polymer hollow shapes, such that the biofilm formation which accumulates therein provide sufficient expansion of the bio-mass available and time reference at the input selector or accommodation modules and wherein thus the level of traces, or recognizable components at any given time is in access of the required level of presence needed or requested to trigger, be recognized, or be analyzed, or promote the functioning and smooth responses of the consume of the biological enhancements and/or polishing made to the water according to the present invention, and wherein only post filtration is needed or involved complimenting prefiltration of the present invention for specific quantity or particle size distribution.

23. A method for production of biologically enhanced drinking liquid or breathing gas, according to any one of the preceding claims, wherein vegetation, or fruits, or plants are irrigated or watered with biologically enhanced products produced according to the present invention and wherein liquid is extracted from said vegetables and fruits for enhancement of the production substrate qualities or thickness through diluting and subtracting, adding or concentrating said processing components to include also parts of the fruit and vegetables, therein or in any combination thereof.

24. A method for production of biologically enhanced drinking liquid or breathing gas, according to any one of the preceding claims, wherein the last stage in a production site using integrated production effort involving use of the method, is a juice dispenser or syrup dispenser, or mass production for bottling plant or factory.

25. A method for production of biologically enhanced drinking liquid or breathing gas, according to any one of the preceding claims, wherein at least one external module for processing, or feeding, or monitoring, or adding, or mixing is being used in the production in order to expand the processing powers of the devices using the method.

26. A method for production of biologically enhanced drinking liquid or breathing gas, according to any one of the preceding claims, wherein a separation or sorting of a predetermined volume or quantity of components made available by photochemistry is done by centrifuge, or mechanical separation interface, or by weight or quantity measured or reference on-line or off line, by ultra-sound transient cavitation from about 1 Hz to about 126 kilo-hertz or by photonic dissociation using peak power or adequate average energy, and wherein the energy required for dissociation is greater then the energy used for inactivation, thus enhancing the controllability of quality and quantity of elements therein.

27. A method for production of biologically enhanced drinking liquid or breathing gas, according to any one of the preceding claims, further comprising the step of dissociating said liquids, and/or gasses, and/or solids or combinations, optically in optical dissociation module (FIG. 1, 1-8/a-z, FIGS. 2-12, ref. 1-100) by delivering directly, and/or by transferring by photonic band gapped, or conventional high grade fused silica (HGFS), or quartz, or sapphire, or polymer, or organo-elastomers or combination of optical fibers, or non toxic aerobic liquid light guides, at least one pulsed wave, and/or continuous wave, and/or transient type of light or sound, and/or sonic energies or combination dose having a predetermined fluence rate, average energy, and predetermined intensity, as well as energy density, or combinations from at least one additional radiation unit interconnected or inter-operated, or interfaced to said multi input, output expander interface, and/or linked to said control and diagnostic router (CDR), and/or mechanically, electronically, electro-optically, and/or acoustically and/or sonically or combination (FIGS. 1-12, ref. 1-100) for physical separation of said noxious species predetermined components, to form a predetermined volume of Bio-compound containing a plurality of predetermined traceable proteins, or photo-damage products.

28. A method for production of biologically enhanced drinking liquid or breathing gas, according to any one of the preceding claims, further comprising the step of transferring (FIG. 1, 1-8/a-z, FIGS. 2-12, ref. 1-100), or phase converting and routing. bio-compound enhancements through a predetermined semi transparent or opaque, post filtration unit or module, or membrane or combination having a cut-off from about 0.1 Micron to about 1000 Micron, or by-passing, compounding or mixing or converting said polly-biological compound into the gas state, liquid and/or solid state, and/or adjusting its relative temperature up to steam or gradually, or quickly down through condensation to about ice forming, or heating, cooling or compressing, or for combinations thus making a crystalline for morphemic choice of lattice hosting, or tactically aurora aromatically tracing or calibrating continually or non recurrently said bio-able compound to its biological time referenced or reverenced source of origin, or time stamp, thus each individually made volume of quantified bio able compound or any combination thereof may be processed prior to processing by modules, or spheres of the present invention, by variety of cross platform processes selected from electro-photochemical, and/or electro-optical, and/or electro opto-catalytic processes, and/or Non thermal plasmas, and/or Electron-beam, and/or Y-rays, and/or Supercritical water Oxidation, and/or Electrohydraulic Cavitation & Sonolysis, and/or Photocatalytic Redox Processes, and/or H2O2/UV, and/or H2O2O3, and/or O3/UV, and/or any combinations thereof in proportions, is apportioned to represent an elemental of components, which when traced back in time for a period sufficiently long from about 2 to 18 DNA or RNA replication sequences back, for the purpose of furnishing synchronously said quenching bio able compound for human and animal consumption, by drinking and dripping or by irrigating and or temporarily or permanently, or prominently said compound is being held or distributed for storage and bottling, analysis or for feeding animals, or watering agricultural produce to be later consumed by man updating and enhancing consumer's immune system performance within a predetermined period of time.

29. A method for production of biologically enhanced drinking liquid or breathing gas, according to any one of the preceding claims, further comprising the step of adding or subtracting from the liqued or gas at least one vitamin, and/or nutrient, or coloring, or stabilizers, or minerals, or carbonation, or additional liquids, and/or gas or any combination thereof in groups or individual components.

30. A method for production of biologically enhanced drinking liquid or breathing gas, according to any one of the preceding claims, further comprising an additional step selected from freezing, boiling, vaporizing, liquidating, solidifying, condensing, or concentrating the enhanced liquid or gas produced according to the method, to be diluted or mixed With other additional liquids and gasses produced according to the method.

31. A method for production of biologically enhanced drinking liquid or breathing gas, according to any one of the preceding claims, further comprising the step of photo-chemically highlighting liquid, gas, or a combination thereof, by at least single event wherein multi photon absorption have occurred in time, or at least one event of said time wherein free radicals formation has caused separation, or aberration, or miss-formation of geometry, or photochemical damage products, and/or opto-acoustic physiologically accelerated modifications, or separation of protein products or mix, polished for potently enabling traceable recognition post consumption for immunifying remedy and thus, causing immunifying effects, by producing universal and thus enhanced water platform for consumption, to inform, and update solutions against evolution of noxious species, or said remedy in said waters, may be applied to diseases, and/or infectious threats selected from; influenza, allergies, cold, flu, engine, viruses, bacteria, virus, macrophage, antibody, immune disorders, disease of the immune system which causes misunderstanding of the immune system reactions, and/or luck of vitality and capabilities of immune defense reaction, or for maintaining a healthy portfolio of decisive immune actions, and/or reactions, or any combination thereof.

32. A method for production of biologically enhanced drinking liquid or breathing gas, according to any one of the preceding claims, further comprising the step of selection of proportions from the cells, organs, or groups of the immune system opulent portfolios of defending actions and wherein such elements, or components of the immune system may be triggered, and their populations, or qualities of their species specific abilities are thus enhanced upon consumption, and said species may include, selection of individual, and/or grouped combinations prompting a responsive, or decisive, or interactive pre-programmed, or predetermined, preset, or variably adjusted for the purpose of harnessing the interconnectivity, and interoperability of responses for remedy, and equipping scope of at least one of the organs, and/or cells such as stem cells, and/or myeloid precursor, and/or platelets, and/or eosinophil, and/or neutrophil, and/or basophil, and/or basophilic type of cells, and/or mast cells, and/or macrophage, and/or plasma cell, and/or sytotoxic t cell, and/or suppressor t cell, and/or helper t cell, and/or lymphoid precursor, and/or b cell, and/or monocyte, and/or tonsils and adenoids, and/or upper lymph nodes, and/or appendix, and/or bone marrow, and/or thymus, and/or spleen, and/or peyer's patches, lower lymph nodes, and/or lymphatic vassals, and/or brain, and/or nerve system, and/or aerobic system, and/or non-aerobic system or drinking, breathing, or rubbing, spraying, or drizzling, dripping, and, or irrigating, feeding, and/or supplementing with, or any combinations thereof the enhancements are stored, mixed, or diluted, or added, or aerated with, or vaporized to contain said traceable enhancements in representing agents such as milk, sugar, air for breathing, weat, rice, or vegetables, bear, wine, or liqueurs, fruits, nuts, and seaweed's staffs, meat, food staffs, or beverages, or in vinegar, or fuel, or combinations or the purpose of immunifying end users, producers and providers and consumers post consumption.

33. A method for production of biologically enhanced drinking liquid or breathing gas, according to any one of the preceding claims, further comprising the step of storing in bottles, cardboard containers, capsules, plastic or polymeric storage bags or tanks, containers, or conduits, modules, or a combination thereof wherein said combination, or in parts are having an enhanced length of shelf life, and biocompatible combination therein, increases the length in which liquids and gasses or their combination may be stored temporarily prior to consumption, or permanently, an/or prominently, and once consumed, thus causing positive effects and host of responsive, or decisive, or interactive pre-programmed, or predetermined, preset, or variably adjusted for the purpose of harnessing the interconnectivity, and interoperability of responses for remedy, and equipping scope of at least one of the organs, and/or cells such as stem cells, and/or myeloid precursor, and/or platelets, and/or eosinophil, and/or neutrophil, and/or basophil, and/or basophilic type of cells, and/or mast cells, and/or macrophage, and/or plasma cell, and/or sytotoxic t cell, and/or suppressor t cell, and/or helper t cell, and/or lymphoid precursor, and/or b cell, and/or monocyte, and/or tonsils and adenoids, and/or upper lymph nodes, and/or appendix, and/or bone marrow, and/or thymus, and/or spleen, and/or peyer's patches, lower lymph nodes, and/or lymphatic vassals, and/or brain, and/or nerve system, and/or aerobic system, and/or non-aerobic system.

34. A device for the production of biologically enhanced drinking liquid or breathing gas or a combination thereof according to the method of the present invention as defined in any of the preceding claims, comprising; at least one dissociation-energy generator unit adapted to generate the dissociative resonance of at least one biological, chemical, molecular, atomic, electronic, phononic, constituent within the liquid or gas to be enhanced; at least one chamber or piping means for receiving or for conduction of liquid or gas or a combination thereof wherein said liquid or gas or their combination can be exposed to energy emerging from said dissociation-energy generator unit; at least one control unit having means for determining an effective ratio between the volume of liquid or gas or a combination thereof, and the amount of dissociation energy to which they can be exposed, wherein the effectiveness of said ratio is useful for a dissociation of a quantity at least one of said constituents by said dissociation energy without altering its geometrical integrity for future traceability by the immune system of human or of other creature or of a plant, upon consumption.

35. A device for the production of biologically enhanced drinking liquid or breathing gas or a combination thereof according to claim 34, wherein the biological constituent within the liquid or gas to be enhanced and to which the dissociative resonance of the dissociation-energy generator unit is adapted to, is selected from the group consisting of DNA, RNA, mRNA, lipid membranes or enzime.

36. A device for the production of biologically enhanced drinking liquid or breathing gas or a combination thereof according to claim 34, wherein the dissociation-energy generator unit is comprised of at least one radiation unit containing at least one high intensity source of light; and wherein the control unit has means for determining an effective ratio between the volume of liquid or gas or a combination thereof, and the amount of light radiation to which they can be exposed, wherein the effectiveness of said ratio is useful for a dissociation of at least part of said components by means of said light radiation, without altering their geometrical integrity for future traceability by the immune system of human or of other creature or of a plant, upon consumption.

37. A device for the production of biologically enhanced drinking liquid or breathing gas or a combination thereof according to claim 34, wherein the dissociation-energy generator unit is comprised of at least one mechanical vibration oscillator unit; and wherein the control unit has means for determining an effective ratio between the volume of liquid or gas or a combination thereof, and the amount of mechanical vibration energy to which they can be exposed, wherein the effectiveness of said ratio is useful for a dissociation of DNA or RNA composite components contained within said liquid or gas by means of said mechanical vibrations, without altering their geometrical integrity for future traceability by the immune system of human or of other creature or of a plant, upon consumption.

38. A device for the production of biologically enhanced drinking liquid or breathing gas or a combination thereof according to claim 34, wherein the dissociation-energy generator unit comprises in combination at least one radiation unit containing at least one high intensity source of light, and at least one mechanical vibration oscillator unit; and wherein said liquid or gas or their combination can be exposed to mechanical vibrations emerging from said oscillator unit and simultaneously can be exposed to light energy emerging from said light source, within the chamber or piping means; and wherein the control unit has means for determining effective ratios between the volume of liquid or gas or a combination thereof, the amount and characteristics of mechanical vibration energy to which they can be exposed, and the amount and characteristics of light energy to which they can be exposed, wherein the effectiveness of said ratios is useful for a dissociation of at least part of the predetermined quantity of biomass components having traceable biologically noxious source or origin contained within said liquid or gas, by means of said mechanical vibrations and said light energy, without altering their geometrical integrity for future traceability by the immune system of human or of other creature or of a plant, upon consumption.

39. A device for the production of biologically enhanced drinking liquid or breathing gas or a combination thereof according to claim 38 wherein the mechanical vibration frequency is between 1 Hertz and 1 Tera Hertz, and wherein the light wavelength is between 1 nm and 3600 nm, and wherein their sub mixing properties or rezonance and wherein the total energy density zone or intensity or flouence rate or monotonic or pollyfonic or vibration exatation level, or photo coagulation or agglomeration or transient or static cavitation or sonolysis are cousing dissociative molecular, atomic, electronic effects while keeping the integrity of single or multi component compound thus created according to the method of the present invention especially beneficial for tunning, referencing trigering stimulating enhancing, rehearsing and increasing the sensitivity, oppulance interconnectivity and flush or interoperability or programability, distribution and the flow of positive, accurate decisive actions and reactions of the NHC majure histo compatibility complex type I-II-III the aquired human immune system within a predetermined volume or period of time.

40. A device for the production of biologically enhanced drinking liquid or breathing gas or a combination thereof according to claim 38, further comprising feedback means for calibrating the light radiation properties by using a real time feedback of light originated within the treated liquid or gas as a result of the mechanical vibration activity, or feedback means for calibrating the mechanical vibration properties by using a real time feedback of ultrasonic mechanical vibrations originated within the treated liquid or gas as a result of the light radiation activity, or any combination thereof.

41. A device for the production of biologically enhanced drinking liquid or breathing gas or a combination thereof according to claim 34, wherein the level of oxygen or oxygenated or hydrogen components or biologica oxigen demand (BOD), or chemical oxigen demand (COD), or partical size distribution, or ph, or radox potential, or suspended solids of a liquid or gas or a combination thereof, or conductivity of any of the constituent therein, are being increased or decreased to suit an adequate reaction rate controlled, or catalized, including externally adding, or subtracting oxygen or hydrogen, through out the production procces.

42. A device for the production of biologically enhanced drinking liquid or breathing gas or a combination thereof according to claim 34, wherein the dissociation-energy generator unit includes a radiation unit comprising crystal based lasers, electrical discharge lasers, gas lasers e-beam pumped eximers, free-electron-lasers, electrostatically accelerated-free-electron-lasers, or solid state diode pumped lasers or hybrid combination wherein the pumping architecture or stimulated emission, or population inversion involved of said laser yields from about one electron, per photon, up to about 150 million photons per electron, in the range from about 40 nm, to about 2400 nm.

43. A device for the production of biologically enhanced drinking liquid or breathing gas or a combination thereof according to claim 32, wherein lasers are being used in conjunction with lamps, or flash-lamps, and wherein their time domain, or intensities, or spectral distribution is calibrated for maximizing multiphotons absorption processes, or excitation, or for delivering appropriate dose for the production processes.

44. A device for the production of biologically enhanced drinking liquid or breathing gas or a combination thereof according to claim 34, wherein the piping means includes at least one photonic band gaped aerobic waveguide for transfering liquid, water, air, gas, light, sound, or ultra-sound, such as steady and/or transient cavitation simultaneously or separately, or sequentially or recurrently or cyclically, or non recurrently for the purpose of remote, or direct illumination, or irradiation, or vibrating, of a predetermined volume of liquid or gas or a combination thereof, within a predetermine space, and over a predetermined period of time.

45. A device for the production of biologically enhanced drinking liquid or breathing gas or a combination thereof according to claim 34, wherein a triad-spheroid, electro-triatmic modulator is claimed for the multi tasking, and/or multiprocessing according to the method of the present invention for the production representations, and chemical, or photochemical traces or components which when mixed, added, integrated, or transfused, or fused, or combined together form the compound according to the method of the present invention, and when consumed to reference, strengthen, streamlined, improve the decisiveness in which immune system responses are occurring or being chemically or hydraulically, or geometrically recognized, analyzed or responded to.

46. A device for the production of biologically enhanced drinking liquid or breathing gas or a combination thereof according to claim 34, wherein a biologically enhanced drinking water or liquid produced according to method, is vaporized prior to consumption, bottling, irrigating, or storing, and wherein vapor droplets size vary from about 1 micron, to about 300 micron, and wherein each droplets is containing sufficiently traces or recognizable proteins signature for enhancing the responses of the human immune system post consumption.

47. A device for the production of biologically enhanced drinking liquid or breathing gas or a combination thereof according to claim 34, wherein a modular input selector is disclosed having individual connection to at least a part from a plurality of modules integrated in the device, or to a control and diagnostic router, or to any externally connected module, conduit, or chamber or a combination thereof, and wherein each of said modules is having an input or output, inlet, or outlet, hydraulic, pneumatic inputs, or outputs, and optical, electronical, acoustic, or ultrasonic inputs and outputs, for the purpose of allowing the routing of signals, or flow rates, volumes, quantities to and from each module in a chain, series, parallel, or arrays interface or workstation geometry.

48. A device for the production of biologically enhanced drinking liquid or breathing gas or a combination thereof according to claim 34, wherein before, between, after, or within each module, or any number of modules interconnected together there may be a valve, a tap, an automatically computer controlled shutter, switch, acausto-optical shutter or modulator, a diffuser, a flow reducer, a flow meter, a settler, an additional housing and storage module, or separate inputs to accommodate complimenting substances, liquids, gasses, solids, powders, syrups, flavors, semiconductor coating, TIO2, I.T.O or combination for the purpose of forming free radicals therein by the utilization of photo-catalysis, electro-catalysis, hydrolysis, centrifuges, and mechanical and/or opto acoustic separation, sorting, and photochemical interaction effecting, and enhancing said biologically enhanced and traceable liquid, gas or a combination thereof, produced according to the method.

49. A device for the production of biologically enhanced drinking liquid or breathing gas or a combination thereof according to claim 34, further comprising means for adding at least one vitamin or nutrient or additional complementing, or harmonious, biocompatible or geneto-compatible substrate or a combination thereof, to a predetermined volume or quantity of biologically enhanced product produced according to the method of the present invention, and wherein said addition is aiding, or positively enhancing the carrying, dispensing, release or time release, or distribution in an individual consumer, or producer body improving the inter-operability of the brain, and bon marrow, or organs of the immune system involved in the production and maintenance control and management of the immune system opulent resources such as stem cells, myeloid precursor, platelets, eosinophil, neutrophil, basophil, basophilic or mast cells, macrophage, plasma cell, sytotoxic t cell, suppressor t cell, helper t cell, lymphoid precursor, b cell, monocyte or any combination thereof, thus enhancing the ability of the human immune system to respond decisively and conserve its resources while, already familiarized threats or invaders, or antigens, or combination thereof are dealt with swiftly and correctly for maximum remedy effects.

50. A device for the production of biologically enhanced drinking liquid or breathing gas or a combination thereof according to claim 34, wherein the liquid or gas is being delivered, distributed or transferred through at least one photonic band gaped aerobic waveguide, or photonic crystal wave guide, or pipe or conduit suitable for such delivery and transfer of continues wave, or pulsed wave type of light from about 40 nm to about 2400 nm, at an average energy from about 1 microwatts to about few gigawatts, and energy density from about 1 mj per Cm square to about 80 joules per Cm square, and pulse duration or width is between 1pico-second Ps to about 1 second, and average energy is delivered from around 1 watt to about 100,000 watts, and wherein spectral distribution is programmed for a predetermined dose within a predetermined space, over a predetermined period of time within at least one module, or sphere of the present invention, especially beneficial for the production of biologically enhanced and photo-chemically polished drinking water, or breathing air, liquid or gas according to the method.

51. A device for the production of biologically enhanced drinking liquid or breathing gas or a combination thereof according to claim 34, adapted for the production and photo-polishing of drinking water, further comprising; at least one input selector, pre-filtration unit, bio-mass expansion module, optical inactivation module, optical dissociation module, physical separation and sorting module, post filtration module, phase conversion router, or a combination thereof, and wherein said processing elements include electro-optical, or electro-triatmic features for the processing, or polishing of liquids, gasses, solids, magnetic radiation, electromagnetic radiation, sound generation or source, ultrasound generation or source for transient cavitation, any combination operating serially, sequentially cyclically, recurrently, non recurrently or in combination and wherein the water, or liquids, and gasses are passed through at least one centrifuge, settler, filtration unit or biological expansion module for the provision of extra proteins, or membranes, or photo-damage products forming higher concentrations of compounding volumes facilitating stronger and more concentrated biologically enhanced and photo-chemically polished production efficiencies.

52. A device for the production of biologically enhanced drinking liquid or breathing gas or a combination thereof according to claim 34, wherein the dissociation-energy generator unit is comprised of at least one radiation unit having a high intensity source of light, and said source, operating synchronously, or separately or in unison, or in a hybrid integrated configuration, is emitting in the ultra violet region of the electro magnetic spectrum, or the visible spectrum or the infra-red portion of the spectrum, and wherein at least one photon is absorbed or excite, or reflected, or transposes said liquids or gasses therein for polishing and enhancement facilitating the production of traceable, biologically enhanced drinking water, or breathing air according to the method.

53. A device for the production of biologically enhanced drinking liquid or breathing gas or a combination thereof according to claim 34, wherein the dissociation-energy generator unit is comprised of at least one radiation unit comprising at least one radiation unit having a high intensity source of light from about 40 nm to about 2400 nm, and wherein said light characteristics is continued, or pulsed and wherein pulsed and continued light is mixed or super imposed, or combined or diffused, or stirred, or diffracted, or splits, or transferred, distributed or projected, targeted or reflected directly to any of the processing modules, or spheres or being guided by photonic band gaped, or optical fibers, or waveguides, or liquids wave guide, especially beneficial for the photo-chemically polishing and the production of traceable substrates or drinking water or breathing air, for the photo-polishing of mineral water, spring water and beverages, according to the method.

54. A device for the production of biologically enhanced drinking liquid or breathing gas or a combination thereof according to claim 34, comprising at least one module, or conduit, or chamber wherein optical inactivation, or disinfection, or dissociation, or secretion, or proteolysis, or photo-lysis or particle size reduction is performed, and/or induced for leaving predetermined marks, inception, or encryption, or traces, or signals or remains, or proteins or membranes, or photodamage products according to the method of the present invention, especially beneficial for strengthening the immune system opulent response curves for improved health by triggering organs or cells or combinations of the immune system response portfolio of actions.

55. A device for the production of biologically enhanced drinking liquid or breathing gas or a combination thereof according to claim 34, further comprising at least one pond, container, or reservoir, having a predetermined volume, cross section, or diameter extending over a predetermined space and an aerator connected to it, or in proximity to said pond or container or tank; at least one compressor having pumping circuits or interface for distributing said biological enhanced, and photopolished water, or air into said pond, by aeration, or by dripping, or by mixing, or by dilution, or by addition, or by bubbling the volume in said or container for the provision or introduction of said enhanced water, and air to said volume of the pond, or container for treatment of living fish, animals, and plants in said water and wherein the aerator is immersed, or inserted, dipped or secured to the bottom of the said pond for the provision of said biological enhancements in liquids and gasses within a predetermined space, over a predetermined period of time.

56. A device for the production of biologically enhanced drinking liquid or breathing gas or a combination thereof according to claim 34, further comprising at least one triad spheroid electro-triatmic opto-electronic, acousto-optic modulator, or multi-phase multi processing platform, driven by a plurality of monochromatic CW or PW or quazi-CW single line or multi line lasers to produce a global polichromatic photo-induced chemical reactions or photo catalitic effects, or electro-catalitic products all of which help maintain of the geometrical integrity and shape of individual biological components within a multi-component compound produced according to the method.

57. A device for the production of biologically enhanced drinking liquid or breathing gas or a combination thereof according to claim 34, further comprising means for cooling, heating compressing, or a combination thereof, of liquid, gas or a combination thereof, prior, during or after the dissociation activity.

58. A device for the production of biologically enhanced drinking liquid or breathing gas or a combination thereof according to claim 34, further comprising means for accommodating or pumping a predetermined feed, or biomass suspension or expansion or a source of liquids or gasses or solids or any combination thereof into or through a predetermined conduit or chamber or module or biological settler having a predetermined volume, cross section and a geometry with at least one input and output, or inlet or outlet or combinations of terminals or integrated interfaces for interfacing, or interconnecting, or inter-operating with a control diagnostic router, and/or multi input and output expander with option for onboard concentrators or cylindrical and multi shapes reactors.

59. A device for the production of biologically enhanced drinking liquid or breathing gas or a combination thereof according to claim 34, further comprising means for transferring liquid, gas, solid or a combination thereof, through (FIG. 1, 1-8/a-z, FIGS. 2-12, ref. 1-100) compressing or gating means, or for inter-modulating said liquid, gas, solid, or any combination thereof through a semi opaque pre-filtration unit having a predetermined cut-off or threshold or at least one membrane from about 0.1 Micron to about 1000 Micron, a transmission capacity and cut-off threshold useful for the removal of large particulate materials.

60. A device for the production of biologically enhanced drinking liquid or breathing gas or a combination thereof according to claim 34, additionally comprising means for exposing liquid, or gas, solid, or any combination thereof, for bio-expansion (FIG. 1, 1-8/a-z, FIGS. 2-12, ref. 1-100), and opto-physiological, and opto-acousto enhancements, by means of at least one pulsed wave or continuous wave, or transient type of light or sound energies or combination dose having a predetermined fluence rate, average energy, and predetermined intensity, as well as energy density, or combinations from at least one radiation unit interconnected, and/or inter-operated to at least one multi input, and/or output expander interface, and/or linked to control diagnostic router, and/or directly having a high intensity light source therein, and wherein at least one wavelength of the region from about 40 nm to about 2400 nm and energy from about 1 micro watt to about a few giga-watts interferes, being applied, or geometrically utilized and wherein, at least one beam of light from the said radiation unit is stirred or directed for distribution by at least one optical fibers or photonic crystals and/or photonic band gaped aerobic waveguides or combinations or directly to said liquids, or gases or chamber or conduit or module or any combination thereof or said liquids or gasses, or light, or combination may flow throughout the said guides or directly be compressed or freely flow by gravitation or atmospheric pressure and/or pumping; enhancing and strengthening and expanding repair activity, proteins production, and/or definition yields, and/or by acousto-optic, or sonic, and/or photo-reactivation of the noxious microorganisms contained within said liquid, gas, or any combination thereof, over a predetermined period of time from about 1 Millisecond to about 24 hours.

61. A device for the production of biologically enhanced drinking liquid or breathing gas or a combination thereof according to claim 34, further comprising means for Inactivating said liquids, or gases or solids or any combination thereof by delivering directly, and/or by transferring by photonic band gapped, or conventional high grade fused silica (HGFS), or quartz, or sapphire, or polymer, or organo-elastomers or combination of optical fibers, or non toxic aerobic liquid light guides, at least one pulsed wave, and/or continuous wave, and/or transient type of light or sound, and/or sonic energies or combination dose having a predetermined fluence rate, average energy, and predetermined intensity, as well as energy density, or combinations from at least one additional radiation unit interconnected or inter-operated, or interfaced to said multi input, output expander interface, and/or linked to said control and diagnostic router (CDR) DNA and/or RNA replication sequences of specific noxious species by electro-photochemical, and/or electro-optical, and/or electro opto-catalytic processes (FIG. 1, 1-8/a-z, FIGS. 2-12, ref. 1-100) and/or Non thermal plasmas, and/or Electron-beam, and/or Y-rays, and/or Supercritical water Oxidation, and/or Electrohydraulic Cavitation & Sonolysis, and/or Photocatalytic Redox Processes, and/or H2O2/UV, and/or H2O2/O3, and/or O3/UV, variant oxidation agent and/or any combinations thereof (Fig. 1, 1-8/a-z, FIGS. 2-12, ref. 1-100).

62. A device for the production of biologically enhanced drinking liquid or breathing gas or a combination thereof according to claim 34, having at least one module, sphere, conduit, or chamber that is embedded, integrated, or attached directly or receiving by photonic band gapped, or directly being projected with, illuminated, irradiated, or exposed to electro magnetic radiation from which at least one photon, or beam of light may include wavelength from about 40 nm, to about 2,400 nm, and wherein the spatial characteristics of the delivered light is equalized or distributed spatially, or uniformly forming appropriate high energy density zones from about 1 mj per square centimeter, to about 100 Jouls per squares centimeter, and wherein said light is having a pulsed, or continuos characteristics, and wherein each portion of energy directed at target within said modules, or conduits, or chambers is having time domain definitions of intensity, or fluence rate, or fluence dose, or dose, or peak powers from about 0.10 kilowatt, to about 100 Gigawatts, and wherein said light is hybridly generated or singularly generated from at least one laser, or lamp, or flash lamp, or low, high, medium pressure light sources and fleshes, or any combination thereof and wherein said time domain effecting pulse width or pulse duration, or continues average. energy delivered at color temperature from 100kelvin to about 10 thousand Kelvin and wherein whitelight, or Ultra violet light, or infrared light is involved in producing traceable enhancements which upon consumption positively enhance the immune system which a responsive, or decisive, or interactive pre-programmed, or predetermined, preset, or variably adjusted for the purpose of harnessing the interconnectivity, and interoperability of responses for remedy, and equipping scope of at least one of the organs, and/or cells such as stem cells, and/or myeloid precursor, and/or platelets, and/or eosinophil, and/or neutrophil, and/or basophil, and/or basophilic type of cells, and/or mast cells, and/or macrophage, and/or plasma cell, and/or sytotoxic t cell, and/or suppressor t cell, and/or helper t cell, and/or lymphoid precursor, and/or b cell, and/or monocyte, and/or tonsils and adenoids, and/or upper lymph nodes, and/or appendix, and/or bone marrow, and/or thymus, and/or spleen, and/or peyer's patches, lower lymph nodes, and/or lymphatic vassals, and/or brain, and/or nerve system, and/or aerobic system, and/or non-aerobic system.

63. A device for the production of biologically enhanced drinking liquid or breathing gas or a combination thereof according to claim 34, located, positioned, or integral to a track, helicopter, ship, or plain.

64. A device for the production of biologically enhanced drinking liquid or breathing gas or a combination thereof according to claim 34, comprising at least one unit, module, or sphere, located, positioned, or integral to a track, helicopter, ship, or plain.

65. A device for the production of biologically enhanced drinking liquid or breathing gas or a combination thereof according to claim 34, having processing modules and/or spheres suited for removing toxic noxious species by altering their electronic, chemical, and molecular structures, such that they could be recognized by immune components, organs, or be free of toxic chemicals like Selenium, Bromates, Boron by exposing these chemicals to light pulses having sub-microseconds time duration's, and removing floating particles by filtration, scooping, or settling tanks, conduits or chambers, for cleaning said liquids, and gasses from toxic compounds by pumping clean liquids, and gases from a lower, or higher level physically, and/or physiologically.

66. A device for the production of biologically enhanced drinking liquid or breathing gas or a combination thereof according to claim 34, having processing modules and/or spheres comprising photonic band gap waveguide, a flowing liquid waveguide, or a conventional conduit or chamber having a jet inlet, or flowing liquid waveguide outlet.

67. A device for the production of biologically enhanced drinking liquid or breathing gas or a combination thereof according to claim 34, having processing modules and/or spheres, integral, interfaced, or connected, to a tunable self amplified, self emission, electrostatically accelerated free electron laser, having a magnetic compound concentrator geometry or separate electronic pumping circuitry.

68. A device for the production of biologically enhanced drinking liquid or breathing gas or a combination thereof according to claim 34, having modules and/or spheres driven by a solid state diode pumped Yag laser having peak power, average power, and repetition rate, for efficiently dissociating any specific species M RNAs for later recognition of the type, and potential threat that specie poses on a specific acquired immune system.

69. A use of biologically enhanced drinking liquid or breathing gas or a combination thereof produced according to the method of the present invention as defined in any of the preceding claims, for immunizing living creature or plant.

70. A use of biologically enhanced drinking liquid or breathing gas or a combination thereof according to claim 69, wherein a post noxious bio-compound opto-physiological enhancements are being transferred or routed for programming or positively referancing charged suspension for rehearsing, and stimulating the acquired immune system, or for chemically equipping immune cells and organs of the human body upon consumption of A++ compound over a predetermined period of time.

71. A use of biologically enhanced drinking liquid or breathing gas or a combination thereof according to claim 69, wherein at least one human, chicken, or cow, bird, or reptile, insect, or micro, and/or macro organism is consuming said biologically enhanced liquid or gas by spraying, washing, rubbing, dipping, wetting, or drying or said biologically enhanced liquid or gas is being consumed as food stuffs, still, or bubbling drinks, flavored, or enriched with minerals, or nutrients or oxygenation reducing substances for the purpose of enhancing the immune system through dietary schedules, and wherein said dietary schedules are aligned on time track for updating the immune system to potentially threatening infectious events or pending diseases, or for the purpose of preempting, securing, and making safe generations of crops, and animals, man and plants by consuming, in-taking, drinking, and breathing applying, and vaporizing, cleaning with, or soaking in deep, or shallow volumes, or, said biological enhancements according to the present invention calibrate the immune system through enhanced and accelerated production of immune system elements and/or cells, and or organs, and which a responsive, or decisive, or interactive pre-programmed, or predetermined, preset, or variably adjusted for the purpose of harnessing the interconnectivity, and interoperability of responses for remedy, and equipping scope of at least one of the organs, and/or cells such as stem cells, and/or myeloid precursor, and/or platelets, and/or eosinophil, and/or neutrophil, and/or basophil, and/or basophilic type of cells, and/or mast cells, and/or macrophage, and/or plasma cell, and/or sytotoxic t cell, and/or suppressor t cell, and/or helper t cell, and/or lymphoid precursor, and/or b cell, and/or monocyte, and/or tonsils and adenoids, and/or upper lymph nodes, and/or appendix, and/or bone marrow, and/or thymus, and/or spleen, and/or peyer's patches, lower lymph nodes, and/or lymphatic vassals, and/or brain, and/or nerve system, and/or aerobic system, and/or non-aerobic system and any combination thereof, thus once said biologically or photochemically, or electro-optically enhanced liquids and gasses, water, and/or air is consumed, through electrochemical signaling, recognition, and respective memorized, reaction times and procedures, and strings of the immune system participating in the response, or recognition of traceable substances by the brain is being improved, and wherein each of said elements, organs, or cells of the immune system or combinations is quantized and/or referenced and/or stimulated for smoothing non uniformity in responses, and/or updating mhc type 1, 2, 3 and for increasing vitality, awareness, or sensually stimulating the brain or increasing libido, or effecting the chemistry which underline all human physiological interactions through out each individual point in human, animals, and plants life cycle, and thus providing a beneficial points of reference, or reverence from which the immune system could mount adequate responses decisively, swiftly and in tune to the microcosm of events, and environmental parameters.

72. A use of biologically enhanced drinking liquid or breathing gas or a combination thereof according to claim 69, wherein at least one moving, produced, protruded, promoted, primed, priorities, or complimented, enhanced, strengthened, triggered, or produced, transferred, or distributed, equipped, armed, or assign cell or organ of the immune system resources is being activated, or replicate on consumption of biologically enhanced and photo-chemically polished drinking water, or breathing air produced in accordance with the reaction caused utilizing, and referencing, or reverencing, memorizing, or updating and calibrating immune system responses in a decisive manner for maximizing remedy orientated action by the immune system to invaders, and wherein such organs or cells include complement, macrophage, viruses, lymphokines, t-cell, b-cell, antibodies, killer cells and wherein participating organs in such response to said recognition, or memorizing may include tonsiles, adenoids, lymph nodes, appendix, bone marrow, lymphatic cells and vassals, lymph nodes, peyer's patches spleen thymus, brain, and wherein each intake of the biologically enhanced and photochemical polished volume may update individual mhc type 1, 2, 3, and thus updating their awareness, immune system ability to interact and decisively deal with infection or threatening bacteria, or viruses, or antibodies, or diseases and foreign substances, according to the method.

73. A use of biologically enhanced drinking liquid or breathing gas or a combination thereof according to claim 69, wherein at least one drop, or breathing measure of biological traceable drinking water, or breathing air, a conduit or chamber for storing or holding, transferring, or diluting, concentrating, or adding to said drop, or breathing measure a predetermined amount of water, or air, liquids, or gasses for the provision of high quality, immune system supplement for enhancing immune system in fish, shrimps, prawns, rice fields, wheat fields, aquaculture, dripping and irrigation for vegetation, and fruits, and for the enhancement of water based animals and plants, or for the enhancing of the human immune system opulent response portfolio after consumption or intake of said animals and plants in foods, beverages, and eating supplements.

74. A use of biologically enhanced drinking liquid or breathing gas or a combination thereof according to claim 69, for the management and maintenance of bodily immune system resources through drinking, or irrigating vegetables and fruits, or for animals and for empowering human immune system continuum of libido-pranic type vitalizing reaction or remedy events and reify relevant transport and distribution, production and definition—swiftly, should an invasion occurs by a bacterium, virus, pathogens, or other noxious species, and wherein triggered defense reaction by the immune system, immune organs, immune cells is decisive, swift, takes shorter time span, and automatically updates mhc 1, 2, 3 and/or causing at least one replication, production, equipping, empowering, chemically modifying, or adding, or subtracting, or signaling, or recognizing, or any combination thereof of at least one participant in said immune response decisive action, and said participants include stem cells, and/or myeloid precursor, and/or platelets, and/or eosinophil, and/or neutrophil, and/or basophil, and/or basophilic type of cells, and/or mast cells, and/or macrophage, and/or plasma cell, and/or sytotoxic t cell, and/or suppressor t cell, and/or helper t cell, and/or lymphoid precursor, and/or b cell, and/or monocyte, and/or tonsils and adenoids, and/or upper lymph nodes, and/or appendix, and/or bone marrow, and/or thymus, and/or spleen, and/or peyer's patches, lower lymph nodes, and/or lymphatic vassals, and/or brain, or any combination, therein involving at least one actively charged, or distributed, maintained and stored, released and targeted, or selected components, and sub-components for action, and remedy.

75. A use of biologically enhanced drinking liquid or breathing gas or a combination thereof according to claim 69, for rigerring in advance, a predetermined immune action by selecting traces according to the method of the present invention wherein at least one components, organ, or cell's performances thus, improved by consumption by drinking and breathing said biologically and optophysiological enhancments, and wherein traceable marks are recognized promoting the enhancment of said components of the immune system in fish, animals, humans or combination involving stem cells, and/or myeloid precursor, and/or platelets, and/or eosinophil, and/or neutrophil, and/or basophil, and/or basophilic type of cells, and/or mast cells, and/or macrophage, and/or plasma cell, and/or sytotoxic t cell, and/or suppressor t cell, and/or helper t cell, and/or lymphoid precursor, and/or b cell, and/or monocyte, and/or tonsils and adenoids, and/or upper lymph nodes, and/or appendix, and/or bone marrow, and/or thymus, and/or spleen, and/or peyer's patches, lower lymph nodes, and/or lymphatic vassals, and/or brain.

76. A use of biologically enhanced drinking liquid or breathing gas or a combination thereof according to claim 69, for immunizingly quenching thirst of consumers while improving their species specific responsive, or decisive, or interactive pre-programmed, or predetermined, preset, or variably adjusted for the purpose of harnessing the interconnectivity, and interoperability of responses for remedy, and equipping scope of at least one of the organs, and/or cells such as stem cells, and/or myeloid precursor, and/or platelets, and/or eosinophil, and/or neutrophil, and/or basophil, and/or basophilic type of cells, and/or mast cells, and/or macrophage, and/or plasma cell, and/or sytotoxic t cell, and/or suppressor t cell, and/or helper t cell, and/or lymphoid precursor, and/or b cell, and/or monocyte, and/or tonsils and adenoids, and/or upper lymph nodes, and/or appendix, and/or bone marrow, and/or thymus, and/or spleen, and/or peyer's patches, lower lymph nodes, and/or lymphatic vassals, and/or brain, and/or nerve system, and/or aerobic system, and/or non-aerobic system.

77. A use of biologically enhanced drinking liquid or breathing gas or a combination thereof according to claim 69, for the management of bodily resources and empowering human immune system by when intake by consume occurs (post 1-8/a-z) prompting the rehearsing of end users major histocompatibility complex or acquired immune system opulent portfolio of defense redemption or remedy, especially beneficial in drinks and foods stuffs, forming action ability to react positively and decisively against invaders of a predetermined space over a predetermined period of time.

78. A use of biologically enhanced drinking liquid or breathing gas or a combination thereof according to claim 69, wherein a bottle holding a globally prepared, and/or specifically produced opto-physiological, photochemically polished drinking water, and wherein said water is held, or stored, temporarily, or permanently, or prominently until consumption for the purpose of quenching thirst, and improving the operation integrity of the Human Immune System (HIS), and wherein at least one organ, and/or cell of the immune system is being effected and improved post consunption, and wherein such immune components may include at least enhanced and accelerated production of immune system elements and/or cells, and or organs, and which a responsive, or decisive, or interactive pre-programmed, or predetermined, preset, or variably adjusted for the purpose of harnessing the interconnectivity, and interoperability of responses for remedy, and equipping scope of at least one of the organs, and/or cells such as stem cells, and/or myeloid precursor, and/or platelets, and/or eosinophil, and/or neutrophil, and/or basophil, and/or basophilic type of cells, and/or mast cells, and/or macrophage, and/or plasma cell, and/or sytotoxic t cell, and/or suppressor t cell, and/or helper t cell, and/or lymphoid precursor, and/or b cell, and/or monocyte, and/or tonsils and adenoids, and/or upper lymph nodes, and/or appendix, and/or bone marrow, and/or thymus, and/or spleen, and/or peyer's patches, lower lymph nodes, and/or lymphatic vassals, and/or brain, and/or nerve system, and/or aerobic system, and/or non-aerobic system and any combination thereof, thus once said biologically or photochemically, or electro-optically enhanced liquids and gasses, water, and/or air is consumed, through electrochemical signaling, recognition, and respective memorized, reaction times and procedures, and strings of the immune system participating in the response, or recognition of traceable substances by the brain is being improved, and wherein each of said elements, organs, or cells of the immune system or combinations is quantized and/or referenced and/or stimulated for smoothing non uniformity in responses, and/or updating mhc type 1, 2, 3 and for increasing vitality, awareness, or sensually stimulating the brain or increasing libido, or effecting the chemistry which underline all human physiological interactions through out each individual point in human, animals, and plants life cycle.

79. A use of biologically enhanced drinking liquid or breathing gas or a combination thereof according to claim 69, wherein said enhanced drinking liquid or breathing gas is encapsulated within a gelatin capsule, a carbomer suspension, a drop of water, or in small droplets in a spray or aerators, ionizer, or sprinkler, dripping irrigation or vegetation, plants, fruits, crops, milk, water, mineral water, syrup, wine, vinegar, cream, food additive, food supplement, flavored water, beverages, blood, serum, bodily fluids, and a pH stabilized suspension or mix, or any combination thereof for immunizing living beings.

80. A use of biologically enhanced drinking liquid or breathing gas or a combination thereof according to claim 69, wherein said enhanced drinking liquid or breathing air is cooled or heated, frozen, or vaporized to be taken through drinking, breathing, intravenously or externally, or internally or through inhalation, through digestive action of fruits vegetables, grains, plants flowers, roots for immunizing living beings

81. A use of biologically enhanced drinking liquid or breathing gas or a combination thereof according to claim 69, wherein production of A++ compounds according to the method of the present invention will include a direct connection to a source or mineral water, spring or aquifer, air or air conditioning systems, and/or for sanitation and global treatment systems for domestic, commercial, agricultural or medical infrastructure support means or structural buildings.

82. A use of biologically enhanced drinking liquid or breathing gas or a combination thereof according to claim 69, wherein liquids and gases solids or combinations are exposed to processes according to the methodology of the of present invention, and wherein their surface or enclosing encapsulating skin, or molecular structures, or electronic conductivity or composition, or physical integrity is undamaged, such that processes cording to the methodology of the present invention may be applied in a non invasive fashion, thus treating vegetation, fruits, and agricultural produce, animals and plants containing said liquids, or gases or solids or combination to be enhanced in situ in real time while cellulite growth is in progress, thus treated externally with light and sound or any combination thereof according to the method of the present invention.

83. A use of biologically enhanced drinking liquid or breathing gas or a combination thereof according to claim 69, wherein tumors, cancers, allergic, and allergenic or infectious events or combinations are being treated with biological enhancements according to the method and devices of the present invention which enhance the ability and the opulence of the reaction portfolio of immune systems of living beings.

84. A use of biologically enhanced drinking liquid or breathing gas or a combination thereof according to claim 69, wherein single drops, or droplets having a predetermined droplet size diameter, or density are offered for periodically, cyclically recurrently, or non-recurrently utilization, intake or consumption in form of pills, capsules, time release capsules, gelatin enclosures, carbomer suspensions, mixed or diluted, reduced or expanded for as specific predetermined immune system is treated, or being approached.

85. A use of biologically enhanced drinking liquid or breathing gas or a combination thereof according to claim 69, wherein a cream or lotion, a wash, or conditioner is made or mixed with liquids, gases, solids, vapors, or spray or combinations thereof containing a or predetermined quantity or volume of said biological enhancements according to the present invention.

86. A use of biologically enhanced drinking liquid or breathing gas or a combination thereof according to claim 69, wherein biologically enhanced compounds according to the method of the present invention is being taken by inhalation, inhalators, infused by drinks, food, breathing or air conditioning systems.

87. A use of biologically enhanced drinking liquid or breathing gas or a combination thereof according to claim 69, wherein biologically enhanced compounds according to the method of the present invention is being injected, or applied manually, intravenously, intramuscular, intra sub-cautenauosly, or by suppositories, or by intra cavity, or combinations, or non invasively applied externally for immunization.
